SEQUENCE LISTING (28 sequences)

SEQ ID NO: 1 - protein, 781 aa, Homo sapiens
MATQADLMELDMAMEPDRKAAVSHWQQQSYLDSGIHSGATTTAPSLSGKGNPEEEDVDTSQVLYEWEQGFSQSFTQEQVADIDGQYAMTRAQRVRAAMFPETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMAWNETADLGLDIGAQGEPLGYRQDDPSYRSFHSGGYGQDALGMDPMMEHEMGGHHPGADYPVDGLPDLGHAQDLMDGLPPGDSNQLAWFDTDL

SEQ ID NO: 2 - protein, 2843 aa, Homo sapiens
MAAASYDQLLKQVEALKMENSNLRQELEDNSNHLTKLETEASNMKEVLKQLQGSIEDEAMASSGQIDLLERLKELNLDSSNFPGVKLRSKMSLRSYGSREGSVSSRSGECSPVPMGSFPRRGFVNGSRESTGYLEELEKERSLLLADLDKEEKEKDWYYAQLQNLTKRIDSLPLTENFSLQTDMTRRQLEYEARQIRVAMEEQLGTCQDMEKRAQRRIARIQQIEKDILRIRQLLQSQATEAERSSQNKHETGSHDAERQNEGQGVGEINMATSGNGQGSTTRMDHETASVLSSSSTHSAPRRLTSHLGTKVEMVYSLLSMLGTHDKDDMSRTLLAMSSSQDSCISMRQSGCLPLLIQLLHGNDKDSVLLGNSRGSKEARARASAALHNIIHSQPDDKRGRREIRVLHLLEQIRAYCETCWEWQEAHEPGMDQDKNPMPAPVEHQICPAVCVLMKLSFDEEHRHAMNELGGLQAIAELLQVDCEMYGLTNDHYSITLRRYAGMALTNLTFGDVANKATLCSMKGCMRALVAQLKSESEDLQQVIASVLRNLSWRADVNSKKTLREVGSVKALMECALEVKKESTLKSVLSALWNLSAHCTENKADICAVDGALAFLVGTLTYRSQTNTLAIIESGGGILRNVSSLIATNEDHRQILRENNCLQTLLQHLKSHSLTIVSNACGTLWNLSARNPKDQEALWDMGAVSMLKNLIHSKHKMIAMGSAAALRNLMANRPAKYKDANIMSPGSSLPSLHVRKQKALEAELDAQHLSETFDNIDNLSPKASHRSKQRHKQSLYGDYVFDTNRHDDNRSDNFNTGNMTVLSPYLNTTVLPSSSSSRGSLDSSRSEKDRSLERERGIGLGNYHPATENPGTSSKRGLQISTTAAQIAKVMEEVSAIHTSQEDRSSGSTTELHCVTDERNALRRSSAAHTHSNTYNFTKSENSNRTCSMPYAKLEYKRSSNDSLNSVSSSDGYGKRGQMKPSIESYSEDDESKFCSYGQYPADLAHKIHSANHMDDNDGELDTPINYSLKYSDEQLNSGRQSPSQNERWARPKHIIEDEIKQSEQRQSRNQSTTYPVYTESTDDKHLKFQPHFGQQECVSPYRSRGANGSETNRVGSNHGINQNVSQSLCQEDDYEDDKPTNYSERYSEEEQHEEEERPTNYSIKYNEEKRHVDQPIDYSLKYATDIPSSQKQSFSFSKSSSGQSSKTEHMSSSSENTSTPSSNAKRQNQLHPSSAQSRSGQPQKAATCKVSSINQETIQTYCVEDTPICFSRCSSLSSLSSAEDEIGCNQTTQEADSANTLQIAEIKEKIGTRSAEDPVSEVPAVSQHPRTKSSRLQGSSLSSESARHKAVEFSSGAKSPSKSGAQTPKSPPEHYVQETPLMFSRCTSVSSLDSFESRSIASSVQSEPCSGMVSGIISPSDLPDSPGQTMPPSRSKTPPPPPQTAQTKREVPKNKAPTAEKRESGPKQAAVNAAVQRVQVLPDADTLLHFATESTPDGFSCSSSLSALSLDEPFIQKDVELRIMPPVQENDNGNETESEQPKESNENQEKEAEKTIDSEKDLLDDSDDDDIEILEECIISAMPTKSSRKAKKPAQTASKLPPPVARKPSQLPVYKLLPSQNRLQPQKHVSFTPGDDMPRVYCVEGTPINFSTATSLSDLTIESPPNELAAGEGVRGGAQSGEFEKRDTIPTEGRSTDEAQGGKTSSVTIPELDDNKAEEGDILAECINSAMPKGKSHKPFRVKKIMDQVQQASASSSAPNKNQLDGKKKKPTSPVKPIPQNTEYRTRVRKNADSKNNLNAERVFSDNKDSKKQNLKNNSKVFNDKLPNNEDRVRGSFAFDSPHHYTPIEGTPYCFSRNDSLSSLDFDDDDVDLSREKAELRKAKENKESEAKVTSHTELTSNQQSANKTQAIAKQPINRGQPKPILQKQSTFPQSSKDIPDRGAATDEKLQNFAIENTPVCFSHNSSLSSLSDIDQENNNKENEPIKETEPPDSQGEPSKPQASGYAPKSFHVEDTPVCFSRNSSLSSLSIDSEDDLLQECISSAMPKKKKPSRLKGDNEKHSPRNMGGILGEDLTLDLKDIQRPDSEHGLSPDSENFDWKAIQEGANSIVSSLHQAAAAACLSRQASSDSDSILSLKSGISLGSPFHLTPDQEEKPFTSNKGPRILKPGEKSTLETKKIESESKGIKGGKKVYKSLITGKVRSNSEISGQMKQPLQANMPSISRGRTMIHIPGVRNSSSSTSPVSKKGPPLKTPASKSPSEGQTATTSPRGAKPSVKSELSPVARQTSQIGGSSKAPSRSGSRDSTPSRPAQQPLSRPIQSPGRNSISPGRNGISPPNKLSQLPRTSSPSTASTKSSGSGKMSYTSPGRQMSQQNLTKQTGLSKNASSIPRSESASKGLNQMNNGNGANKKVELSRMSSTKSSGSESDRSERPVLVRQSTFIKEAPSPTLRRKLEESASFESLSPSSRPASPTRSQAQTPVLSPSLPDMSLSTHSSVQAGGWRKLPPNLSPTIEYNDGRPAKRHDIARSHSESPSRLPINRSGTWKREHSKHSSSLPRVSTWRRTGSSSSILSASSESSEKAKSEDEKHVNSISGTKQSKENQVSAKGTWRKIKENEFSPTNSTSQTVSSGATNGAESKTLIYQMAPAVSKTEDVWVRIEDCPINNPRSGRSPTGNTPPVIDSVSEKANPNIKDSKDNQAKQNVGNGSVPMRTVGLENRLNSFIQVDAPDQKGTEIKPGQNNPVPVSETNESSIVERTPFSSSSSSKHSSPSGTVAARVTPFNYNPSPRKSSADSTSARPSQIPTPVNNNTKKRDSKTDSTESSGTQSPKRHSGSYLVTSV

SEQ ID NO: 3 - protein, 862 aa, Homo sapiens
MNIQEQGFPLDLGASFTEDAPRPPVPGEEGELVSTDPRPASYSFCSGKGVGIKGETSTATPRRSDLDLGYEPEGSASPTPPYLKWAESLHSLLDDQDGISLFRTFLKQEGCADLLDFWFACTGFRKLEPCDSNEEKRLKLARAIYRKYILDNNGIVSRQTKPATKSFIKGCIMKQLIDPAMFDQAQTEIQATMEENTYPSFLKSDIYLEYTRTGSESPKVCSDQSSGSGTGKGISGYLPTLNEDEEWKCDQDMDEDDGRDAAPPGRLPQKLLLETAAPRVSSSRRYSEGREFRYGSWREPVNPYYVNAGYALAPATSANDSEQQSLSSDADTLSLTDSSVDGIPPYRIRKQHRREMQESVQVNGRVPLPHIPRTYRVPKEVRVEPQKFAEELIHRLEAVQRTREAEEKLEERLKRVRMEEEGEDGDPSSGPPGPCHKLPPAPAWHHFPPRCVDMGCAGLRDAHEENPESILDEHVQRVLRTPGRQSPGPGHRSPDSGHVAKMPVALGGAASGHGKHVPKSGAKLDAAGLHHHRHVHHHVHHSTARPKEQVEAEATRRAQSSFAWGLEPHSHGARSRGYSESVGAAPNASDGLAHSGKVGVACKRNAKKAESGKSASTEVPGASEDAEKNQKIMQWIIEGEKEISRHRRTGHGSSGTRKPQPHENSRPLSLEHPWAGPQLRTSVQPSHLFIQDPTMPPHPAPNPLTQLEEARRRLEEEEKRASRAPSKQRYVQEVMRRGRACVRPACAPVLHVVPAVSDMELSETETRSQRKVGGGSAQPCDSIVVAYYFCGEPIPYRTLVRGRAVTLGQFKELLTKKGSYRYYFKKVSDEFDCGVVFEEVREDEAVLPVFEEKIIGKVEKVD

SEQ ID NO: 4 - protein, 843 aa, Homo sapiens
MSSAMLVTCLPDPSSSFREDAPRPPVPGEEGETPPCQPGVGKGQVTKPMSVSSNTRRNEDGLGEPEGRASPDSPLTRWTKSLHSLLGDQDGAYLFRTFLEREKCVDTLDFWFACNGFRQMNLKDTKTLRVAKAIYKRYIENNSIVSKQLKPATKTYIRDGIKKQQIDSIMFDQAQTEIQSVMEENAYQMFLTSDIYLEYVRSGGENTAYMSNGGLGSLKVVCGYLPTLNEEEEWTCADFKCKLSPTVVGLSSKTLRATASVRSTETVDSGYRSFKRSDPVNPYHIGSGYVFAPATSANDSEISSDALTDDSMSMTDSSVDGIPPYRVGSKKQLQREMHRSVKANGQVSLPHFPRTHRLPKEMTPVEPATFAAELISRLEKLKLELESRHSLEERLQQIREDEEREGSELTLNSREGAPTQHPLSLLPSGSYEEDPQTILDDHLSRVLKTPGCQSPGVGRYSPRSRSPDHHHHHHSQYHSLLPPGGKLPPAAASPGACPLLGGKGFVTKQTTKHVHHHYIHHHAVPKTKEEIEAEATQRVHCFCPGGSEYYCYSKCKSHSKAPETMPSEQFGGSRGSTLPKRNGKGTEPGLALPAREGGAPGGAGALQLPREEGDRSQDVWQWMLESERQSKPKPHSAQSTKKAYPLESARSSPGERASRHHLWGGNSGHPRTTPRAHLFTQDPAMPPLTPPNTLAQLEEACRRLAEVSKPPKQRCCVASQQRDRNHSATVQTGATPFSNPSLAPEDHKEPKKLAGVHALQASELVVTYFFCGEEIPYRRMLKAQSLTLGHFKEQLSKKGNYRYYFKKASDEFACGAVFEEIWEDETVLPMYEGRILGKVERID

SEQ ID NO: 5 - protein, 420 aa, Homo sapiens
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKVFRPRTPPEAIALCSRLLEYTPTARLTPLEACAHSFFDELRDPNVKLPNGRDTPALFNFTTQELSSNPPLATILIPPHARIQAAASTPTNATAASDANTGDRGQTNNAASASASNST

SEQ ID NO: 6 - protein, 907 aa, Homo sapiens
MDTSRLGVLLSLPVLLQLATGGSSPRSGVLLRGCPTHCHCEPDGRMLLRVDCSDLGLSELPSNLSVFTSYLDLSMNNISQLLPNPLPSLRFLEELRLAGNALTYIPKGAFTGLYSLKVLMLQNNQLRHVPTEALQNLRSLQSLRLDANHISYVPPSCFSGLHSLRHLWLDDNALTEIPVQAFRSLSALQAMTLALNKIHHIPDYAFGNLSSLVVLHLHNNRIHSLGKKCFDGLHSLETLDLNYNNLDEFPTAIRTLSNLKELGFHSNNIRSIPEKAFVGNPSLITIHFYDNPIQFVGRSAFQHLPELRTLTLNGASQITEFPDLTGTANLESLTLTGAQISSLPQTVCNQLPNLQVLDLSYNLLEDLPSFSVCQKLQKIDLRHNEIYEIKVDTFQQLLSLRSLNLAWNKIAIIHPNAFSTLPSLIKLDLSSNLLSSFPITGLHGLTHLKLTGNHALQSLISSENFPELKVIEMPYAYQCCAFGVCENAYKISNQWNKGDNSSMDDLHKKDAGMFQAQDERDLEDFLLDFEEDLKALHSVQCSPSPGPFKPCEHLLDGWLIRIGVWTIAVLALTCNALVTSTVFRSPLYISPIKLLIGVIAAVNMLTGVSSAVLAGVDAFTFGSFARHGAWWENGVGCHVIGFLSIFASESSVFLLTLAALERGFSVKYSAKFETKAPFSSLKVIILLCALLALTMAAVPLLGGSKYGASPLCLPLPFGEPSTMGYMVALILLNSLCFLMMTIAYTKLYCNLDKGDLENIWDCSMVKHIALLLFTNCILNCPVAFLSFSSLINLTFISPEVIKFILLVVVPLPACLNPLLYILFNPHFKEDLVSLRKQTYVWTRSKHPSLMSINSDDVEKQSCDSTQALVTFTSSSITYDLPPSSVPSPAYPVTESCHLSSVAFVPCL

SEQ ID NO: 7 - protein, 783 aa, Homo sapiens
MSGGHQLQLAALWPWLLMATLQAGFGRTGLVLAAAVESERSAEQKAIIRVIPLKMDPTGKLNLTLEGVFAGVAEITPAEGKLMQSHPLYLCNASDDDNLEPGFISIVKLESPRRAPRPCLSLASKARMAGERGASAVLFDITEDRAAAEQLQQPLGLTWPVVLIWGNDAEKLMEFVYKNQKAHVRIELKEPPAWPDYDVWILMTVVGTIFVIILASVLRIRCRPRHSRPDPLQQRTAWAISQLATRRYQASCRQARGEWPDSGSSCSSAPVCAICLEEFSEGQELRVISCLHEFHRNCVDPWLHQHRTCPLCMFNITEGDSFSQSLGPSRSYQEPGRRLHLIRQHPGHAHYHLPAAYLLGPSRSAVARPPRPGPFLPSQEPGMGPRHHRFPRAAHPRAPGEQQRLAGAQHPYAQGWGLSHLQSTSQHPAACPVPLRRARPPDSSGSGESYCTERSGYLADGPASDSSSGPCHGSSSDSVVNCTDISLQGVHGSSSTFCSSLSSDFDPLVYCSPKGDPQRVDMQPSVTSRPRSLDSVVPTGETQVSSHVHYHRHRHHHYKKRFQWHGRKPGPETGVPQSRPPIPRTQPQPEPPSPDQQVTRSNSAAPSGRLSNPQCPRALPEPAPGPVDASSICPSTSSLFNLQKSSLSARHPQRKRRGGPSEPTPGSRPQDATVHPACQIFPHYTPSVAYPWSPEAHPLICGPPGLDKRLLPETPGPCYSNSQPVWLCLTPRQPLEPHPPGEGPSEWSSDTAEGRPCPYPHCQVLSAQPGSEEELEELCEQAV

SEQ ID NO: 8 - protein, 936 aa, Homo sapiens
MRPRSGGRPGATGRRRRRLRRRPRGLRCSRLPPPPPLPLLLGLLLAAAGPGAARAKETAFVEVVLFESSPSGDYTTYTTGLTGRFSRAGATLSAEGEIVQMHPLGLCNNNDEEDLYEYGWVGVVKLEQPELDPKPCLTVLGKAKRAVQRGATAVIFDVSENPEAIDQLNQGSEDPLKRPVVYVKGADAIKLMNIVNKQKVARARIQHRPPRQPTEYFDMGIFLAFFVVVSLVCLILLVKIKLKQRRSQNSMNRLAVQALEKMETRKFNSKSKGRREGSCGALDTLSSSSTSDCAICLEKYIDGEELRVIPCTHRFHRKCVDPWLLQHHTCPHCRHNIIEQKGNPSAVCVETSNLSRGRQQRVTLPVHYPGRVHRTNAIPAYPTRTSMDSHGNPVTLLTMDRHGEQSLYSPQTPAYIRSYPPLHLDHSLAAHRCGLEHRAYSPAHPFRRPKLSGRSFSKAACFSQYETMYQHYYFQGLSYPEQEGQSPPSLAPRGPARAFPPSGSGSLLFPTVVHVAPPSHLESGSTSSFSCYHGHRSVCSGYLADCPGSDSSSSSSSGQCHCSSSDSVVDCTEVSNQGVYGSCSTFRSSLSSDYDPFIYRSRSPCRASEAGGSGSSGRGPALCFEGSPPPEELPAVHSHGAGRGEPWPGPASPSGDQVSTCSLEMNYSSNSSLEHRGPNSSTSEVGLEASPGAAPDLRRTWKGGHELPSCACCCEPQPSPAGPSAGAAGSSTLFLGPHLYEGSGPAGGEPQSGSSQGLYGLHPDHLPRTDGVKYEGLPCCFYEEKQVARGGGGGSGCYTEDYSVSVQYTLTEEPPPGCYPGARDLSQRIPIIPEDVDCDLGLPSDCQGTHSLGSWGGTRGPDTPRPHRGLGATREEERALCCQARALLRPGCPPEEAGAVRANFPSALQDTQESSTTATEAAGPRSHSADSSSPGA

SEQ ID NO: 9 - protein, 1613 aa, Homo sapiens
MGAVLRSLLACSFCVLLRAAPLLLYANRRDLRLVDATNGKENATIVVGGLEDAAAVDFVFSHGLIYWSDVSEEAIKRTEFNKTESVQNVVVSGLLSPDGLACDWLGEKLYWTDSETNRIEVSNLDGSLRKVLFWQELDQPRAIALDPSSGFMYWTDWGEVPKIERAGMDGSSRFIIINSEIYWPNGLTLDYEEQKLYWADAKLNFIHKSNLDGTNRQAVVKGSLPHPFALTLFEDILYWTDWSTHSILACNKYTGEGLREIHSDIFSPMDIHAFSQQRQPNATNPCGIDNGGCSHLCLMSPVKPFYQCACPTGVKLLENGKTCKDGATELLLLARRTDLRRISLDTPDFTDIVLQLEDIRHAIAIDYDPVEGYIYWTDDEVRAIRRSFIDGSGSQFVVTAQIAHPDGIAVDWVARNLYWTDTGTDRIEVTRLNGTMRKILISEDLEEPRAIVLDPMVGYMYWTDWGEIPKIERAALDGSDRVVLVNTSLGWPNGLALDYDEGKIYWGDAKTDKIEVMNTDGTGRRVLVEDKIPHIFGFTLLGDYVYWTDWQRRSIERVHKRSAEREVIIDQLPDLMGLKATNVHRVIGSNPCAEENGGCSHLCLYRPQGLRCACPIGFELISDMKTCIVPEAFLLFSRRADIRRISLETNNNNVAIPLTGVKEASALDFDVTDNRIYWTDISLKTISRAFMNGSALEHVVEFGLDYPEGMAVDWLGKNLYWADTGTNRIEVSKLDGQHRQVLVWKDLDSPRALALDPAEGFMYWTEWGGKPKIDRAAMDGSERTTLVPNVGRANGLTIDYAKRRLYWTDLDTNLIESSNMLGLNREVIADDLPHPFGLTQYQDYIYWTDWSRRSIERANKTSGQNRTIIQGHLDYVMDILVFHSSRQSGWNECASSNGHCSHLCLAVPVGGFVCGCPAHYSLNADNRTCSAPTTFLLFSQKSAINRMVIDEQQSPDIILPIHSLRNVRAIDYDPLDKQLYWIDSRQNMIRKAQEDGSQGFTVVVSSVPSQNLEIQPYDLSIDIYSRYIYWTCEATNVINVTRLDGRSVGVVLKGEQDRPRAVVVNPEKGYMYFTNLQERSPKIERAALDGTEREVLFFSGLSKPIALALDSRLGKLFWADSDLRRIESSDLSGANRIVLEDSNILQPVGLTVFENWLYWIDKQQQMIEKIDMTGREGRTKVQARIAQLSDIHAVKELNLQEYRQHPCAQDNGGCSHICLVKGDGTTRCSCPMHLVLLQDELSCGEPPTCSPQQFTCFTGEIDCIPVAWRCDGFTECEDHSDELNCPVCSESQFQCASGQCIDGALRCNGDANCQDKSDEKNCEVLCLIDQFRCANGQCIGKHKKCDHNVDCSDKSDELDCYPTEEPAPQATNTVGSVIGVIVTIFVSGTVYFICQRMLCPRMKGDGETMTNDYVVHGPASVPLGYVPHPSSLSGSLPGMSRGKSMISSLSIMGGSSGPPYDRAHVTGASSSSSSSTKGTYFPAILNPPPSPATERSHYTMEFGYSSNSPSTHRSYSYRPYSYRHFAPPTTPCSTDVCDSDYAPSRRMTSVATAKGYTSDLNYDSEPVPPPPTPRSQYLSAEENYESCPPSPYTERSYSHHLYPPPPSPCTDSS

SEQ ID NO: 10 - protein, 707 aa, Homo sapiens
MNQELLSVGSKRRRTGGSLRGNPSSSQVDEEQMNRVVEEEQQQQLRQQEEEHTARNGEVVGVEPRPGGQNDSQQGQLEENNNRFISVDEDSSGNQEEQEEDEEHAGEQDEEDEEEEEMDQESDDFDQSDDSSREDEHTHTNSVTNSSSIVDLPVHQLSSPFYTKTTKMKRKLDHGSEVRSFSLGKKPCKVSEYTSTTGLVPCSATPTTFGDLRAANGQGQQRRRITSVQPPTGLQEWLKMFQSWSGPEKLLALDELIDSCEPTQVKHMMQVIEPQFQRDFISLLPKELALYVLSFLEPKDLLQAAQTCRYWRILAEDNLLWREKCKEEGIDEPLHIKRRKVIKPGFIHSPWKSAYIRQHRIDTNWRRGELKSPKVLKGHDDHVITCLQFCGNRIVSGSDDNTLKVWSAVTGKCLRTLVGHTGGVWSSQMRDNIIISGSTDRTLKVWNAETGECIHTLYGHTSTVRCMHLHEKRVVSGSRDATLRVWDIETGQCLHVLMGHVAAVRCVQYDGRRVVSGAYDFMVKVWDPETETCLHTLQGHTNRVYSLQFDGIHVVSGSLDTSIRVWDVETGNCIHTLTGHQSLTSGMELKDNILVSGNADSTVKIWDIKTGQCLQTLQGPNKHQSAVTCLQFNKNFVITSSDDGTVKLWDLKTGEFIRNLVTLESGGSGGVVWRIRASNTKLVCAVGSRNGTEETKLLVLDFDVDMK

SEQ ID NO: 11 - protein, 619 aa, Homo sapiens
MPQLNGGGGDDLGANDELISFKDEGEQEEKSSENSSAERDLADVKSSLVNESETNQNSSSDSEAERRPPPRSESFRDKSRESLEEAAKRQDGGLFKGPPYPGYPFIMIPDLTSPYLPNGSLSPTARTLHFQSGSTHYSAYKTIEHQIAVQYLQMKWPLLDVQAGSLQSRQALKDARSPSPAHIVSNKVPVVQHPHHVHPLTPLITYSNEHFTPGNPPPHLPADVDPKTGIPRPPHPPDISPYYPLSPGTVGQIPHPLGWLVPQQGQPVYPITTGGFRHPYPTALTVNASMSRFPPHMVPPHHTLHTTGIPHPAIVTPTVKQESSQSDVGSLHSSKHQDSKKEEEKKKPHIKKPLNAFMLYMKEMRAKVVAECTLKESAAINQILGRRWHALSREEQAKYYELARKERQLHMQLYPGWSARDNYGKKKKRKRDKQPGETNEHSECFLNPCLSLPPITDLSAPKKCRARFGLDQQNNWCGPCRRKKKCVRYIQGEGSCLSPPSSDGSLLDSPPPSPNLLGSPPRDAKSQTEQTQPLSLSLKPDPLAHLSMMPPPPALLLAEATHKASALCPNGALDLPPAALQPAAPSSSIAQPSTSSLHSHSSLAGTQPQPLSLVTKSLE

SEQ ID NO: 12 - DNA, 24 bp, Artificial
  CTNNB1-MiSeq-F
tcaatgggtcatatcacagattct

SEQ ID NO: 13 - DNA, 20 bp, Artificial
  CTNNB1-MiSeq-R
tcctcttcctcaggattgcc

SEQ ID NO: 14 - DNA, 21 bp, Artificial
  Axin2-qPCR-F
tgactctccttccagatccca

SEQ ID NO: 15 - DNA, 19 bp, Artificial
  Axin2-qPCR-R
tgcccacactaggctgaca

SEQ ID NO: 16 - DNA, 20 bp, Artificial
  Jun-qPCR-F
ccttctacgacgatgccctc

SEQ ID NO: 17 - DNA, 22 bp, Artificial
  Jun-qPCR-R
ggttcaaggtcatgctctgttt

SEQ ID NO: 18 - DNA, 19 bp, Artificial
  Wnt16-qPCR-F
gcaggctgtcgccaagtta

SEQ ID NO: 19 - DNA, 22 bp, Artificial
  Wnt16-qPCR-R
gtctgcctctggtctttttctc

SEQ ID NO: 20 - DNA, 21 bp, Artificial
  Lef1-qPCR-F
tgtttatcccatcacgggtgg

SEQ ID NO: 21 - DNA, 21 bp, Artificial
  Lef1-qPCR-R
catggaagtgtcgcctgacag

SEQ ID NO: 22 - DNA, 20 bp, Artificial
  Gapdh-qPCR-F
tggatttggacgcattggtc

SEQ ID NO: 23 - DNA, 21 bp, Artificial
  Gapdh-qPCR-R
tttgcactggtacgtgttgat

SEQ ID NO: 24 - DNA, 20 bp, Artificial
  Actb-qPCR-F
ggctgtattcccctccatcg

SEQ ID NO: 25 - DNA, 22 bp, Artificial
  Actb-qPCR-R
ccagttggtaacaatgccatgt

SEQ ID NO: 26 - DNA, 58 bp, Artificial
  Ezh2-shRNA-1
ccggcggctcctctaaccatgtttactcgagtaaacatggttagaggagccgtttttg

SEQ ID NO: 27 - DNA, 58 bp, Artificial
  Ezh2-shRNA-2
ccgggcacaagtcatcccgttaaagctcgagctttaacgggatgacttgtgctttttg

SEQ ID NO: 28 - DNA, 57 bp, Artificial
  shRNA-Ctrl
ccgggcgcgatagcgctaataatttctcgagaaattattagcgctatcgcgcttttt

(19) United States
(12) Patent Application Publication
(10) Pub. No.: US 2023/0404985 A1
(43) Pub. Date: Dec. 21, 2023

(54) METHODS OF TREATING TUMORS AND CANCERS HAVING DYSREGULATED WNT SIGNALING PATHWAYS

(71) Applicant: ICAHN SCHOOL OF MEDICINE AT MOUNT SINAI, New York, NY (US)

(72) Inventors: Ernesto GUCCIONE, New York, NY (US); Amaia LUJAMBIO, New York, NY (US); Arvin DAR, New York, NY (US); Alexander RIALDI, New York, NY (US); Mary E. DUFFY, New York, NY (US); Alexander P. SCOPTON, New York, NY (US); Pedro MOLINA-SANCHEZ, New York, NY (US); Slim MZOUGHI, New York, NY (US)

(21) Appl. No.: 18/034,965

(22) PCT Filed: Nov. 2, 2021
(86) PCT No.: PCT/US2021/057746

§ 371 Date: May 2, 2023

Related U.S. Application Data
(60) Provisional application No. 63/108,728, filed on Nov. 2, 2020.

(51) Int. Cl. A61K 31/44; A61K 45/06; A61P 35/00

(52) U.S. Cl. CPC A61K 31/44; A61K 45/06; A61P 35/00

(57) ABSTRACT

The present disclosure relates to methods of treating tumors and cancers having dysregulated Wnt signaling pathways with compounds of Formula (I) having the following structure: (I) or a stereoisomer, pharmaceutically acceptable salt, oxide, or solvate thereof, where X is a halogen and R is a phenyl substituted with a perfluoroalkane. Also disclosed is a method of treating a tumor, which involves contacting a tumor comprising cytoplasmic EZH2 with a kinase inhibitor compound under conditions effective to treat the tumor.

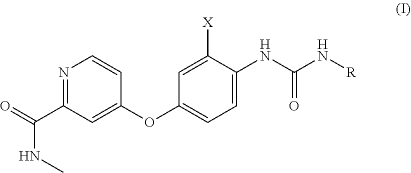

This application claims the priority benefit of U.S. Provisional Patent Application Ser. No. 63/108,728, filed Nov. 2, 2020, which is hereby incorporated by reference in its entirety.

This invention was made with government support under 1F32CA247414-01 and CA249204 awarded by National Institutes of Health. The government has certain rights in the invention.

### FIELD

The present disclosure relates to methods of treating tumors and cancers having dysregulated Wnt signaling pathways.

### BACKGROUND

Hepatocellular carcinoma (“HCC”) is the most common form of primary liver cancer, which is currently the fourth leading cause of cancer-related death globally (Yang et al., “A Global View of Hepatocellular Carcinoma: Trends, Risk, Prevention and Management,” Nat. Rev. Gastroenterol. & Hepatol. 16(10):589-604 (2019) and Llovet et al., “Hepatocellular Carcinoma,” Nat. Rev. Dis. Primers 7(1):6 (2021)). Incidence of this cancer type in the United States has tripled over the last forty years, and unlike most cancers, there has been a continued increase in mortality rate (Llovet et al., “Hepatocellular Carcinoma,” Nat. Rev. Dis. Primers 7(1):6 (2021)). Major risk factors, such as chronic viral hepatitis, alcohol use disorder and non-alcoholic fatty liver disease (NAFLD) in the context of obesity and diabetes, have high global prevalence (Llovet et al., “Hepatocellular Carcinoma,” Nat. Rev. Dis. Primers 7(1):6 (2021)), underlining the increased importance of liver cancer as a public health concern. Advanced-stage HCC has a poor prognosis, with the most recently approved frontline therapy, atezolizumab plus bevacizumab, leading to a median survival of 19.2 months (Finn et al., “Atezolizumab plus Bevacizumab in Unresectable Hepatocellular Carcinoma,” N. Engl. J. Med. 382(20):1894-1905 (2020)).

Before this, and for over a decade, the mainstay of therapy has been multi-targeted kinase inhibitors, including the drug sorafenib with reported median survival of 11-14 months (Llovet et al., “Hepatocellular Carcinoma,” Nat. Rev. Dis. Primers 7(1):6 (2021)). Overall response rates to current HCC drugs remain below 30% (Llovet et al., “Hepatocellular Carcinoma,” Nat. Rev. Dis. Primers 7(1):6 (2021)), highlighting the need for more effective and targeted therapeutics to treat this cancer type.

HCC has been genetically characterized as heterogeneous; however, several key drivers of disease have been identified (Harding et al., “Prospective Genotyping of Hepatocellular Carcinoma: Clinical Implications of Next-Generation Sequencing for Matching Patients to Targeted and Immune Therapies,” Clin. Cancer Res. 25(7):2116-2126 (2018); Ally et al., “Comprehensive and Integrative Genomic Characterization of Hepatocellular Carcinoma,” Cell 169(7):1327-1341 (2017); and Hoshida et al., “Integrative Transcriptome Analysis Reveals Common Molecular Subclasses of Human Hepatocellular Carcinoma,” Cancer Res. 69(18):7385-7392 (2009)). Mutations in β-catenin (encoded by CTNNB1), which occur in ˜30% of cases, are of particular therapeutic interest (Llovet et al., “Hepatocellular Carcinoma,” Nat. Rev. Dis. Primers 7(1):6 (2021); Harding et al., “Prospective Genotyping of Hepatocellular Carcinoma: Clinical Implications of Next-Generation Sequencing for Matching Patients to Targeted and Immune Therapies,” Clin. Cancer Res. 25(7):2116-2126 (2018); Ally et al., “Comprehensive and Integrative Genomic Characterization of Hepatocellular Carcinoma,” Cell 169(7):1327-1341 (2017); and Hoshida et al., “Integrative Transcriptome Analysis Reveals Common Molecular Subclasses of Human Hepatocellular Carcinoma,” Cancer Res. 69(18):7385-7392 (2009)). These mutations induce a dominant oncogenic addiction loop, which is thus far undruggable (Llovet et al., “Hepatocellular Carcinoma,” Nat. Rev. Dis. Primers 7(1):6 (2021); Harding et al., “Prospective Genotyping of Hepatocellular Carcinoma: Clinical Implications of Next-Generation Sequencing for Matching Patients to Targeted and Immune Therapies,” Clin. Cancer Res. 25(7):2116-2126 (2018); Ally et al., “Comprehensive and Integrative Genomic Characterization of Hepatocellular Carcinoma,” Cell 169(7):1327-1341 (2017); and Hoshida et al., “Integrative Transcriptome Analysis Reveals Common Molecular Subclasses of Human Hepatocellular Carcinoma,” Cancer Res. 69(18):7385-7392 (2009)).

Additionally, CTNNB1 mutations are associated with tumor immune exclusion, and patients with these mutations have been shown to respond poorly to immunotherapy (Harding et al., “Prospective Genotyping of Hepatocellular Carcinoma: Clinical Implications of Next-Generation Sequencing for Matching Patients to Targeted and Immune Therapies,” Clin. Cancer Res. 25(7):2116-2126 (2018); Sia et al., “Liver Cancer Cell of Origin, Molecular Class, and Effects on Patient Prognosis,” Gastroenterology 152(4):745-761 (2017); and Bassaganyas et al., “Copy-Number Alteration Burden Differentially Impacts Immune Profiles and Molecular Features of Hepatocellular Carcinoma,” Clin. Cancer Res. 26(23): 6350-6361 (2020)). This resistance has also been recapitulated in murine disease models (Ruiz de Galarreta et al., “β-Catenin Activation Promotes Immune Escape and Resistance to Anti-PD-1 Therapy in Hepatocellular Carcinoma,” Cancer Discov. 9(8):1124-1141 (2019)). The vast majority of CTNNB1 mutations inactivate N-terminal phosphodegron sites, causing stabilization of the protein that enables dysregulated transcriptional activation of several downstream pathways (Jung et al., “Wnt Signaling in Cancer: Therapeutic Targeting of Wnt Signaling beyond (3-Catenin and the Destruction Complex,” Exp. Mol. Med. 52(2):183-191 (2020)).

The present disclosure is directed to overcoming these and other deficiencies in the art.

### SUMMARY

One aspect of the present disclosure relates to a method of treating a tumor having a dysregulated Wnt signaling pathway. This method involves contacting a tumor having a dysregulated Wnt signaling pathway with a compound of Formula (I) having the following structure:

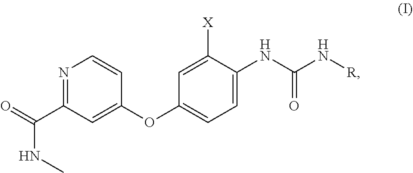

or a stereoisomer, pharmaceutically acceptable salt, oxide, or solvate thereof, wherein
X is a halogen and
R is a phenyl substituted with a perfluoroalkane
   under conditions effective to treat the tumor.

Another aspect of the present disclosure relates to a method of treating a cancer having a dysregulated Wnt signaling pathway in a subject in need thereof. This method involves administering to the subject a compound of Formula (I) having the following structure:

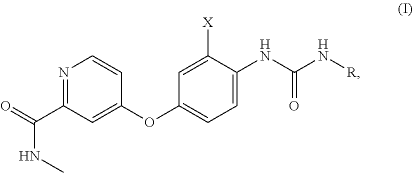

or a stereoisomer, pharmaceutically acceptable salt, oxide, or solvate thereof, wherein
X is a halogen and
R is a phenyl substituted with a perfluoroalkane
   under conditions effective to treat the subject for the cancer.

A further aspect of the present application relates to a method of treating a tumor. This method involves contacting a tumor comprising cytoplasmic EZH2 with a kinase inhibitor compound under conditions effective to treat the tumor.

Given the lack of selective and mutant-specific β-catenin inhibitors or known drug targets without significant on-target toxicity for the Wnt pathway (Jung et al., “Wnt Signaling in Cancer: Therapeutic Targeting of Wnt Signaling beyond β-Catenin and the Destruction Complex,” Exp. Mol. Med. 52:183-191 (2020), which is hereby incorporated by reference in its entirety), small molecules that display specific cell toxicity in models of mutant β-catenin were identified. Through chemical genetic screens focused on genetically-engineered tumor organoids, the Examples of the present disclosure below demonstrate the identification of a selective antagonist of β-catenin-mutant HCC that is effective in vivo and in human-derived model systems. This compound was termed “WNTinib” based on a multiomics strategy that identifies downregulation of oncogenic Wnt signaling via inhibition of KIT/MAPK and downstream activation of EZH2 as the mechanism of action.

### BRIEF DESCRIPTION OF THE DRAWINGS

FIG. 1 is chromatography spectra data for one embodiment of a compound of Formula (I) named WNTinib1 or APS-8-100-2 (40) (4-(3-fluoro-4-(3-(4-(perfluoroethyl)phenyl)ureido)phenoxy)-N-methylpicolinamide).

FIG. 2 is spectra showing ¹H NMR (400 MHz) data for one embodiment of a compound of Formula (I) named WNTinib1 or APS-8-100-2 (40) (4-(3-fluoro-4-(3-(4-(perfluoroethyl)phenyl)ureido)phenoxy)-N-methylpicolinamide).

FIG. 3 is chromatography spectra data for one embodiment of a compound of Formula (I) named WNTinib2 or APS-8-50-2 (36) (4-(3-fluoro-4-(3-(4-(perfluoropropan-2-yl)phenyl)ureido)phenoxy)-N-methylpicolinamide).

FIG. 4 is spectra showing ¹H NMR (400 MHz) data for one embodiment of a compound of Formula (I) named WNTinib2 or APS-8-50-2 (36) (4-(3-fluoro-4-(3-(4-(perfluoropropan-2-yl)phenyl)ureido)phenoxy)-N-methylpicolinamide).

FIGS. 5A-5F show that chemical genetic screens in tumor organoids yield WNTinib as a selective antagonist of CTNNB1-mutated HCC. FIG. 5A (top) is a schematic illustration showing a probe set that was derived from sorafenib and regorafenib, with points of diversification highlighted by R, which are specified for each analog in FIG. 5C. FIG. 5A (bottom) shows the viability of murine HCC organoid models (below: Brightfield and histology) treated with the probe set or HCC-approved compounds. Dosage was set to 5 μM, and endpoint viability was measured after 3 days. Dots represent the average of two biological replicates. FIG. 5B shows the complete chemical structures of WNTinib and 8-50-2. FIG. 5C (left) shows IC₅₀ values for probe set and HCC-approved compounds in MYC-CTNNB1 tumor organoids, MYC-Tp53 tumor organoids, and primary human hepatocytes (mpPHH). Values obtained from two biological replicates. FIG. 5C (right) shows WNT reporter expression levels in MYC-CTNNB1 tumor organoids treated with the same compounds. Values obtained from three biological replicates [mean, SEM]. R column illustrates the cap group diversity of analogs. FIGS. 5D-5F show IC₅₀ values for sorafenib, 8-50-2, or WNTinib in human HCC cell lines (FIG. 5D), primary human HCC cell lines (FIG. 5E), and primary human HCC organoids (FIG. 5F). CTNNB1 or WNT pathway mutations noted in rightmost column. Values obtained from three biological replicates.

FIGS. 6A-6H show that WNTinib induces EZH2 activity to drive the suppression of essential gene networks in CTNNB1-mutated HCC. FIG. 6A-6C are Volcano plots depicting phosphoproteomic (FIG. 6A), transcriptomic (FIG. 6B), and orepigenomic H3K27me3 (FIG. 6C) changes elicited by WNTinib inMYC-CTNNB1 tumor organoids (left) or MYC-Tp53 tumor organoids (right) as compared to DMSO. Inset: pathway enrichment terms associated with significantly regulated phosphoproteins, transcripts, or H3K27me3 peaks. WNTinib was used at 1 μM for 24 hours. Values obtained from two biological replicates. FIG. 6D shows time course of ATF2 and EZH2 phosphorylation in MYC-CTNNB1 tumor organoids treated with DMSO, SB202190 (10 μM), sorafenib (10 μM), or WNTinib (1 μM) for 48 hours. Western blots measure endogenous proteins as indicated. FIG. 6E shows cytoplasmic and nuclear fractions of total and phospho (pT367) EZH2 from MYC-CTNNB1 (left) and MYC-Tp53(right) tumor organoids treated with DMSO, sorafenib (10 μM), or WNTinib (1 μM) for 24 hours. Tubulin and histone H3 used as fractionation controls. FIG. 6F shows IC₅₀ curves for sorafenib (top) or WNTinib (bottom) in MYC-CTNNB1 tumor organoids depleted for EZH2. Inset: western blot depicting depletion efficiency. Values obtained from three biological replicates [mean, SEM]. FIGS. 6G-6H show results of combination treatment matrices for MYC-CTNNB1 tumor organoids treated with WNTinib (left) or sorafenib (right) and compounds targeting EZH2 (top: MS1943 degrader; bottom: GSK343; SAM competitive inhibitor). Tumor organoids were first treated with MS1943 or GSK343 for 3 days, followed by co-treatment with sorafenib or WNTinib for an additional 3 days. Heatmap displays averages from two biological replicates. Combination index (CI) values were calculated for each column and averaged across each matrix.

FIGS. 7A-7J show that WNTinib utilizes unique polypharmacology to regulate the EZH2-WNT axis. FIG. 7A shows kinome selectivity for sorafenib, regorafenib, 8-50-2, and WNTinib. Y-axis indicates the number of kinases that each compound inhibits at >65%, as profiled using KINOME scan. FIG. 7B shows live cell target engagement IC₅₀ values for sorafenib, regorafenib, 8-50-2, and WNTinib on clinically relevant receptor tyrosine kinases, cytoplasmic kinases, and non-kinases. Above: RNA expression of targets in MYC-CTNNB1 and MYC-Tp53 tumor organoids. Heatmap displays averages from three biological replicates.

FIG. 7C shows model of c-KIT bound to WNTinib; the inactive type II conformation enables binding through the para-C₂F5 group, which is highlighted with a red sphere. FIG. 7D Left: Phosphorylation of EZH2 is regulated downstream of a RTK, KIT, PDE6D signaling axis with negative feedback mediated through cytoplasmic kinases, including BRAF and p38. Middle: Sorafenib inhibition on upstream targets is mitigated due to release of negative feedback signaling through direct binding on BRAF and p38. Right: WNTinib strongly down-regulates phospho-EZH2 due to inhibition of targets and removal of anti-target inhibition, thereby avoiding compensatory feedback. As a result, unphosphorylated EZH2 localizes to the nucleus to selectively repress the transcription of WNT targets. FIG. 7E shows combination treatment matrices for MYC-CTNNB1 tumor organoids treated with sorafenib (right) or WNTinib (left) and the KIT ligand SCF. Tumor organoids were treated for 3 days. Heatmap displays averages from two biological replicates. Combination index (CI) values were calculated for each column and averaged across each matrix. FIG. 7F shows a western blot depicting the modulation of pT367 EZH2 by increasing titration of SCF in MYC-CTNNB1 tumor organoids treated with sorafenib (10 μM), 8-50-2 (1 μM), or WNTinib (1 μM) for 24 hours. FIGS. 7G-7H show combination treatment matrices for MYC-CTNNB1 tumor organoids treated with sorafenib (right) or WNTinib (left) and the BRAF inhibitor dabrafenib (FIG. 7G), or the p38 inhibitor SB202190 (FIG. 7H). Tumor organoids were treated for 3 days. Heatmap displays averages from two biological replicates. Combination index (CI) values were calculated for each column and averaged across each matrix. FIG. 7I shows a western blot depicting the modulation of pT367 EZH2 by WNTinib in combination with either dabrafenib (10 PM), SB202190 (10 μM), or the two compounds together. Tumor organoids were treated for 24 hours. FIG. 7J shows WNT reporter expression levels in SNU398 cells treated with WNTinib alone or in combination with dabrafenib or SB202190 for 24 hours. Values obtained from three technical replicates [mean, SEM]. Significant differences between groups (as compared to WNTinib alone) indicated by asterisks. *P<0.05, ***P<0.0005, as calculated with an ANOVA with Tukey test for multiple comparisons (F (7, 16)=379).

FIGS. 8A-8H show that WNTinib outperforms clinical compounds across in vivo models of HCC. FIG. 8A shows dose escalation of WNTinib in C57BL/6J mice. Animals were dosed every day via oral gavage for 2 weeks. Percentage change in body weight noted in brackets (N=5 animals per group; mean, SEM). FIG. 8B shows 72-hour pharmacokinetic curves of WNTinib, sorafenib, and regorafenibin BALB/c animals. Animals were dosed at 20 mg/kg via oral gavage. Together, FIG. 8A and FIG. 8B indicate that significant exposures of WNTinib are well-tolerated in animals. FIG. 8C, Left: tumor volume plot in MYC-CTNNB1 tumor organoid allografts treated with WNTinib or clinical compounds (N=5-10 animals per group; mean, SEM). FIG. 8C, Right: waterfall plot of individual tumor volume changes with treatments. Significant differences between groups indicated by asterisks. *P<0.05, as calculated with an ANOVA with Tukey test for multiple comparisons (F (5, 36)=3.349). FIG. 8D is as in FIG. 8C, but using MYC-Tp53 tumor organoid allografts (N=5 animals per group; mean, SEM). Significant differences between groups indicated by asterisks. *P<0.05, ***P<0.0005, as calculated with an ANOVA with Tukey test for multiple comparisons ((F (5, 36)=3.485). For FIG. 8C and FIG. 8D, animals were dosed via daily oral gavage using 30 mg/kg of respective compounds. Dosing started when tumor volumes reached ˜100 mm³. FIG. 8E shows a tumor volume plot in MYC-CTNNB1 tumor organoid allografts treated with WNTinib or sorafenib (N=9-11 animals per group; mean, SEM). Significant differences between groups indicated by asterisks. **P<0.005, ** *P<0.0005, as calculated with an ANOVA with Tukey test for multiple comparisons (F (14,216)=4.272). Animals were dosed via daily oral gavage using 20 mg/kg of WNTinib and 30 mg/kg of sorafenib. Dosing started when tumor volumes reached ˜350 mm³. FIG. 8F shows quantitative PCR expression of WNT target genes in tumors derived from mice in panele (N=3 per group). *P<0.05, as calculated with paired t-tests. FIG. 8G shows a western blot depicting the modulation of pT367 EZH2 in tumors derived from mice in FIG. 8E (N=3 per group). FIG. 8H shows hydrodynamic tail vein model of CTNNB1-mutated HCC (MYC-lucOS; CTNNB1) treated with vehicle, sorafenib, or WNTinib. Percent survival shown, and log-rank P values indicated (as compared to vehicle). Kinase inhibitors were started 7 days post-injection and dosed at 20 mg/kg (WNTinib) or 30 mg/kg (sorafenib)-5 days on and 2 days off.

FIGS. 9A-9I show chemical genetic screens in tumor organoids yield WNTinib as a selective antagonist of CTNNB1-mutated HCC. FIG. 9A shows the base structures of sorafenib and regorafenib were used as starting points for kinase inhibitor development. FIG. 9B shows the required perfluoroalkyl-substituted aniline building blocks were obtained in a single step from aniline. FIG. 9C shows that in cases where isocyanates were not commercially available, the required acyl imidazole intermediates were generated in situ from an aniline and N,N′-carbonyldiimidazole. FIG. 9D shows that in the final inhibitor generating step, the urea linker component was formed via reaction of a commercially available isocyanate or in situ formed acyl imidazole with a core aniline corresponding to sorafenib (X═H) or regorafenib (X═F). FIG. 9E shows key interactions and predicted bind pose of sorafenib and regorafenib analogs. Points of diversification are highlighted as X and R, which are specified for each analog in FIG. 9D. FIGS. 9F-9I show IC₅₀ curves of WNTinib (FIG. 9F), 8-50-2 (FIG. 9G), sorafenib (FIG. 9H), and regorafenib (FIG. 9I) in murine HCC organoids used in FIG. 5A. Values obtained with three biological replicates [mean, SEM].

FIGS. 10A-10M show WNTinib induces EZH2 activity to drive the suppression of essential gene networks in CTNNB1-mutated HCC. FIG. 10A shows principle component analysis of phosphoproteomics displaying MYC-CTNNB1 and MYC-Tp53 tumor organoids treated or not with WNTinib, as related to FIG. 6A. FIGS. 10B-10C show interaction networks and pathway enrichment for the significantly up and downregulated phosphoproteins in the MYC-CTNNB1 (FIG. 10B) and MYC-Tp53 (FIG. 10C) models treated with WNTinib. Strength of interactions denoted by STRING P value. Proteins driving pathway enrichment shown in boxes. FIGS. 10D-10E show clustered heatmap of the combined score for kinase-substrate predictions for the top substrates (y-axis) and kinases (x-axis) modulated by WNTinib inMYC-CTNNB1 (FIG. 10D) and MYC-Tp53 (FIG. 10E) tumor organoids. Pathway enrichment for both kinases and substrates was done using STRING. Substrates driving enrichment shown in boxes; EZH2 highlighted gray. FIG. 10F-10G Top: principle component analyses (above) of RNA-sequencing displaying MYC-CTNNB1 (FIG. 10F) or MYC-Tp53 (FIG. 10G) tumor organoids treated or not with WNTinib, as related to FIG. 6B. Bottom: gProfiler pathway enrichment for the up and downregulated genes. FIG. 10H shows quantitative PCR validation of RNA-sequencing performed in MYC-CTNNB1 tumor organoids treated with 1 μM of WNTinib for 72 hours, as related to FIG. 6B. Values obtained with three biological replicates [mean, SEM]. Significant differences between groups indicated by asterisks. *P<0.05, as calculated with paired t-tests. FIG. 10I shows gene set enrichment analysis for significantly regulated genes in both MYC-CTNNB1 and MYC-Tp53 tumor organoids treated with WNTinib. The top five negatively and positively associated gene sets were chosen for visualization. FIG. 10J shows a schematic of phospho-site regulation of EZH2 by WNTinib in the MYC-CTNNB1 tumor organoids. Predicted kinases for each site shown in boxes. FIG. 10K shows a western blot depicting the modulation of pT367 EZH2 by WNTinib (1 μM) or sorafenib (10 μM) in the four tumor organoid models used in FIG. 5A. Tumor organoids were treated for 48 hours. FIG. 10L shows quantitative PCR validation for EZH2 shRNA-mediated depletion in MYC-CTNNB1 tumor organoids, as related to FIG. 6F. Values obtained with three biological replicates [mean, SEM]. Significant differences between groups indicated by asterisks. *P<0.05, **P<0.005, as calculated with paired t-tests. FIG. 10M show RNA expression levels of genes in MYC-CTNNB1 tumor organoids depleted for EZH2 and treated with DMSO, sorafenib (10 μM), or WNTinib (1 μM). Genes are classified as being described PRC2 targets or not. Values obtained with three biological replicates [mean, SEM]. Significant differences between groups indicated by asterisks. *P<0.05, **P<0.005, as calculated with paired t-tests.

FIGS. 11A-H show that WNTinib utilizes unique polypharmacology to regulate the EZH2-WNT axis. FIGS. 11A-11D show trees depicting the kinome inhibition profiles of WNTinib (FIG. 11A), 8-50-2 (FIG. 11B), sorafenib (FIG. 11C), and regorafenib (FIG. 11D). FIG. 11E shows IC₅₀ curves of WNTinib (left) or sorafenib (right) in MYC-CTNNB1 tumor organoids stably transduced with MKK6 (S207E, T211E), as compared to parental organoids. Inset: western blot validation for MKK6 overexpression. Values obtained with two biological replicates [mean, SEM]. FIG. 11F is as in FIG. 11E, but in SNU398 cells. FIG. 11G shows WNT reporter expression levels in SNU398 cells stably transduced with MKK6 (S207E, T211E), as compared to parental cells. Cells were treated with WNTinib, 8-50-2, sorafenib, or regorafenib at increasing concentrations for 24 hours. Values obtained from three technical replicates [mean, SEM]. Significant differences (as compared to parental conditions) indicated by asterisks. *P<0.05, ***P<0.0005, as calculated with paired t-tests. FIG. 11H shows a western blot of EZH2 T367 phosphorylation in parental MYC-CTNNB1 and MKK6 (S207E, T211E) overexpressing organoids 24 hours after DMSO, 10 μM sorafenib, 1 μM 8-50-2, or 1 μM WNTinib treatment.

FIGS. 12A-12C show that WNTinib outperforms clinical compounds across in vivo models of HCC. FIGS. 12A-12B show dose escalation of WNTinib in BALB/c mice (FIG. 12A) and BALB/c/nude mice (FIG. 12B). Animals were dosed every day via oral gavage for 2 weeks. Percentage change in body weight noted in brackets (N=5 animals per group; mean, SEM). FIG. 12C shows images of C57BL/6J mice treated with either vehicle or WNTinib for extended periods of time. WNTinib-treated animals present mosaic patterns of grey hair.

FIGS. 13A-13E show results pertaining to colorectal cancer. FIG. 13A shows a TiterGlo assay to assess the viability of the wild type (WT) healthy intestinal organoids and isogenic CRC line (AKS: APCKO::KRASG12D::SMAD4KO) 72 hours post treatment with WNTinib. FIG. 13B shows Brightfield microscopy images of WT organoids treated with DMSO and AKS tumoroids treated with DMSO or WNTinib (3 μM) for 72 hrs. FIG. 13C shows qPCR analysis of the indicated marker genes from organoids shown in FIG. 13B. Data are column/gene normalized on a 0-100 scale with 0 being the lowest expression level and 100 the highest. FIG. 13D show full dose-response analysis of WNTinib's effects on the indicated organoid and tumoroid lines; viability was quantified using the TiterGlo assay after 72 hours. WT intestinal organoids are used as a control. FIG. 13E shows tissues obtained from a study to test WNTinib at 60 mg/kg and 120 mg/kg through oral gavage administration over the course of 14 days (experimental period) in Blab/c mice. Brain, heart, esophagus, large intestine do not reveal pathological changes. Images of the intestine are shown as an example.

FIGS. 14A-14G show results pertaining the use of WNTinib in combination with immunotherapy. FIG. 14A shows a hydrodynamic tail vein mouse model of MYC-lucOS; CTNNB1 mutant HCC. C57/BL6 mice were randomized into treatment groups using IVIS imaging to quantify tumor size based on luminescence signal intensity. After randomization, kinase inhibitors were dissolved in 25% cremaphor:ethanol in water and administered orally (30 mg/kg sorafenib, 20 mg/kg WNTinib) beginning 7 days after injection following 5 days on/2 days off dosing schedule. Immunotherapy (anti-PD-1) was administered via intraperitoneal injection on days 11, 13, and 15 after injection at 200 μg. Log-rank p-values for survival in drug treatment groups to vehicle group and median survival are shown. Mice were assigned to different treatment groups as follows: vehicle (25), sorafenib (10), aPD-1 (15), WNTinib (25), and WNTinib+aPD-1 (25). FIG. 14B shows tumor volume plot in MYC-CTNNB1 tumor organoid allografts treated with vehicle, anti-IgG control, anti-PD-1, sorafenib, sorafenib+anti-PD-1, WNTinib or WNTinib+anti-PD-1 (N=9-11 animals per group; mean, SEM). Significant differences between groups indicated by asterisks. ** P<0.005, *** P<0.0005, as calculated with an ANOVA with Tukey test for multiple comparisons. Animals were dosed via daily oral gavage using 20 mg/kg of WNTinib and 30 mg/kg of sorafenib. Immunotherapy (anti-PD-1, indicated as ICB in the figure) was administered via intraperitoneal injection on days 3, 5, and 7 post treatment initiation at 200 μg. Dosing started when tumor volumes reached ˜350 mm³. FIG. 14C shows the waterfall plots of tumor volume change from the starting baseline. WNTinib and WNTinib+anti PD-1 were the only two treatments able to induce tumor regression. FIG. 14D shows the median survival plots for the same experiment. Immunotherapy (anti-PD-1, indicated as ICB in the figure) was additionally administered (3 injections at 2 days intervals) via intraperitoneal injection at days 15 and 36 at 200 μg. FIG. 14E shows the quantitative PCR expression of WNT target genes in tumors derived from mice in FIG. 14A (N=3 per group). * P<0.05, as calculated with paired t-tests. FIG. 14F shows the western blot depicting the modulation of pT367 EZH2 in tumors derived from mice in FIG. 14A (N=3 per group). FIG. 14G shows the immune cell profiling of tumors from mice in FIG. 14A. 3 animals per indicated groups were assessed using spectral cytometry panels for both lymphocytes and myeloid cells. The analysis was conducted six days after the start of dosing (days 1-6: kinase inhibitors; days 3 and 5: immunotherapy). * P<0.05, ** P<0.005, as conducted with a ANOVA with Tukey test.

### DETAILED DESCRIPTION

### Definitions

Unless otherwise indicated, the definitions and embodiments described in this and other sections are intended to be applicable to all embodiments and aspects of the present application herein described for which they are suitable as would be understood by a person skilled in the art.

Singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure. In another example, reference to “a compound” includes both a single compound and a plurality of different compounds.

The term “about” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” depends on the particular system under study, and can be readily appreciated by one of ordinary skill in the art.

The term “and/or” as used herein means that the listed items are present, or used, individually or in combination. In effect, this term means that “at least one of” or “one or more” of the listed items is used or present.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “involving”, “having”, and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps. In embodiments or claims where the term comprising (or the like) is used as the transition phrase, such embodiments can also be envisioned with replacement of the term “comprising” with the terms “consisting of” or “consisting essentially of.” The methods, kits, systems, and/or compositions of the present disclosure can comprise, consist essentially of, or consist of, the components disclosed.

The terms “express” and “expression” mean allowing or causing the information in a gene or DNA sequence to become produced, for example producing an RNA or a protein by activating the cellular functions involved in transcription and/or translation of a corresponding gene or DNA sequence. A DNA sequence is expressed in or by a cell to form an “expression product” such as an RNA or a protein. The expression product itself, e.g., the resulting protein, may also be said to be “expressed” by the cell. An expression product can be characterized as intracellular, extracellular or transmembrane.

The term “indels” refers to small duplications, deletions, and/or insertions which involve anywhere between one to ten nucleotides and in other embodiments, the indels are duplications, deletions, and/or insertions involving up to 50 nucleotides. In some embodiments, the indel is a one nucleotide deletion indel. In other embodiments, the indel is a two nucleotide deletion indel. In other embodiments, the indel is a three nucleotide deletion indel, a four nucleotide deletion indel, a five nucleotide deletion indel, a six nucleotide deletion indel, a seven nucleotide deletion indel, an eight nucleotide deletion indel, a nine nucleotide deletion indel, or a ten nucleotides deletion indel. In other embodiments, the indel is a one nucleotide insertion indel, a two nucleotide insertion indel, a three nucleotide insertion indel, a four nucleotide insertion indel, a five nucleotide insertion indel, a six nucleotide insertion indel, a seven nucleotide insertion indel, an eight nucleotide insertion indel, a nine nucleotide insertion indel, or a ten nucleotide insertion indel. In the embodiments where the indels are up to 50 nucleotides, the duplication, deletion, or insertion can be up to 50 nucleotides, e.g., 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 nucleotides.

Certain terms employed in the specification, examples, and claims are collected herein. Unless defined otherwise, all technical and scientific terms used in this disclosure have the same meanings as commonly understood by one of ordinary skill in the art to which this disclosure belongs.

Preferences and options for a given aspect, feature, embodiment, or parameter of the disclosure should, unless the context indicates otherwise, be regarded as having been disclosed in combination with any and all preferences and options for all other aspects, features, embodiments, and parameters of the disclosure.

The Wnt Signaling Pathway

The present disclosure relates to methods of treating tumors and cancers having a dysregulated Wnt signaling pathway.

In β-catenin dependent (canonical) Wnt signaling, absence of Wnt ligands leads to phosphorylation of β-catenin by the destruction complex, which contains the scaffold proteins Axin and APC and the kinases GSK3β and CK1α (see, e.g., Zhan et al., “Wnt Signaling in Cancer,” Oncogene 36:1461-1473 (2017), which is hereby incorporated by reference in its entirety). In this state, β-catenin is phosphorylated by GSK3β, ubiquitinated by β-TrCP200, and targeted for proteasomal degradation. In the absence of nuclear β-catenin, a repressive complex containing T cell factor (TCF), lymphoid enhancer factor (LEF), and transducing-like enhancer protein (TLE/Groucho) recruits histone deacetylases (HDACs) to repress target genes. Conversely, nuclear β-catenin forms an active complex with LEF and TCF proteins by displacing TLE/Groucho complexes and recruitment of histone modifying co-activators such as CBP/p300, BRG1, BCL9, and Pygo.

As used herein, the term “dysregulated Wnt signaling pathway” refers to a Wnt signaling pathway comprising a component that interferes with canonical Wnt signaling. For example, the dysregulated Wnt signaling pathway may comprise a mutation in one or more of the β-catenin destruction complex proteins, e.g., β-catenin, Axin, adenomatous polyposis coli (APC), glycogen synthase kinase 3 (GSK3β), and casein kinase 1 (CK1a), upstream co-regulators of the pathway (e.g., ZNRF3/RNF43, LGR5, LRP6), or downstream co-regulators (e.g., FBXW, TCF7L2) (see, e.g., Kimelman & Xu, “β-Catenin Destruction Complex: Insights and Questions from a Structural Perspective,” Oncogene 25:7482-7491 (2006), which is hereby incorporated by reference in its entirety).

The dysregulation of Wnt signaling is associated with various neoplastic diseases (Jung et al., “Wnt Signaling in Cancer: Therapeutic Targeting of Wnt Signaling beyond β-Catenin and the Destruction Complex,” Exp. Mol. Med. 52(2):183-191 (2020), which is herein incorporated by reference in its entirety).

Using focused chemical libraries based on kinase inhibitor scaffolds, small molecule inhibitors of the Wnt signaling pathway having the structure of Formula (I) according to the present disclosure have been identified.

Compounds According to the Present Disclosure

Compounds of Formula (I) of the present disclosure have the following structure:

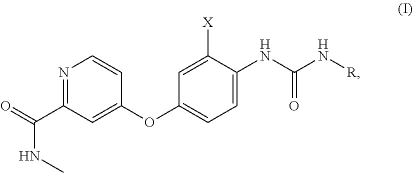

or a stereoisomer, pharmaceutically acceptable salt, oxide, or solvate thereof, where X is a halogen and R is a phenyl substituted with a perfluoroalkane.

As used herein, the term “halogen” means fluoro, chloro, bromo, or iodo. In some embodiments of the compounds of Formula (I), X is fluoro (F).

The term “perfluoralkane” means a fluorocarbon, which is a hydrocarbon having hydrogen atoms replaced by fluorine atoms. Perfluoroalkanes are made up of carbon and fluorine atoms only, and are fully saturated. A perfluoroalkane can be arranged in a linear, branched, cyclic, or polycyclic shape.

The phenyl may be substituted with perflouroalkane in any one or more of the ortho, meta, and para positions. In some embodiments, the phenyl is substituted with a single perflouroalkane in the para position.

In some embodiments, compounds of Formula (I) are as described herein, with the proviso that the compounds of Formula (I) do not include regorafenib.

In some embodiments, compounds of Formula (I) are as described herein, with the proviso that R is a perfluoroalkane other than CF₃.

In some embodiments of compounds of formula (I), R is a phenyl substituted with a perfluoroalkane comprising two or more carbon atoms.

In some embodiments of compounds of Formula (I), R is a phenyl substituted with a single perfluoroalkane compound and the phenyl has no other substituents.

In some embodiments, the perflouroalkane is selected from C₂F₅, and C₃F₇.

In some embodiments, the perfluoroalkane is C₂F₅. One non-limiting exemplary compound of Formula (I) where R is a phenyl substituted with a C₂F₅ perfluoralkane is as follows:

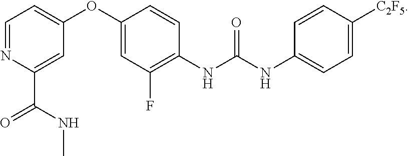

In some embodiments, the perfluoroalkane is C₃F₇. One non-limiting exemplary compound of Formula (I) where R is a phenyl substituted with a C₃F₇ perfluoralkane is as follows:

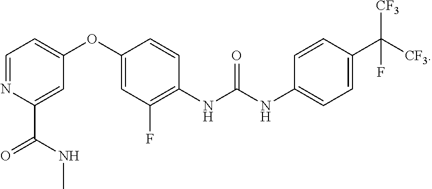 [SMILES: CNC(=O)c1cc(Oc2ccc(NC(=O)Nc3ccc(C(F)(C(F)(F)F)C(F)(F)F)cc3)c(F)c2)ccn1]

The term “substituted” means that one or more hydrogens on a designated atom is replaced with a selection from the indicated group, provided that the designated atom's normal valency is not exceeded.

Compounds described herein (e.g., compounds of Formula (I)) include the prodrugs, the pharmaceutically acceptable salts, the oxides, and the solvates, e.g. hydrates, where the context so permits.

Compounds described herein may contain one or more asymmetric centers and may thus give rise to enantiomers, diastereomers, and other stereoisomeric forms. Each chiral center may be defined, in terms of absolute stereochemistry, as (R)- or (S)-. The present application is meant to include all such possible isomers, as well as mixtures thereof, including racemic and optically pure forms. Optically active (R)- and (S)-, (−)- and (+)-, or (D)- and (L)-isomers may be prepared using chiral synthons or chiral reagents, or resolved using conventional techniques. All tautomeric forms are also intended to be included.

As would be understood by a person of ordinary skill in the art, the recitation of “a compound” is intended to include salts, oxides, solvates, and inclusion complexes of that compound as well as any stereoisomeric form, or a mixture of any such forms of that compound in any ratio. Thus, in accordance with some embodiments of the application, a compound as described herein, including in the contexts of pharmaceutical compositions, methods of treatment, and compounds per se, is provided as the salt form.

The term “solvate” refers to a compound in the solid state where molecules of a suitable solvent are incorporated in the crystal lattice. A suitable solvent for therapeutic administration is physiologically tolerable at the dosage administered. Examples of suitable solvents for therapeutic administration are ethanol and water. When water is the solvent, the solvate is referred to as a hydrate. In general, solvates are formed by dissolving the compound in the appropriate solvent and isolating the solvate by cooling or using an antisolvent. The solvate is typically dried or azeotroped under ambient conditions.

Inclusion complexes are described in Remington, The Science and Practice of Pharmacy, 19th Ed. 1:176-177 (1995), which is hereby incorporated by reference in its entirety. The most commonly employed inclusion complexes are those with cyclodextrins, and all cyclodextrin complexes, natural and synthetic, are specifically encompassed by the present application.

The term “pharmaceutically acceptable salt” refers to salts prepared from pharmaceutically acceptable non-toxic acids or bases including inorganic acids and bases and organic acids and bases. The term “pharmaceutically acceptable” means it is, within the scope of sound medical judgment, suitable for use in contact with the cells of humans and lower animals without undue toxicity, irritation, allergic response, and the like, and are commensurate with a reasonable benefit/risk ratio.

Compounds of Formula (I) may be prepared as set forth in Scheme 1, as follows. Reaction between an aniline (1 or 2) and N,N-carbonyldiimidazole (CDI) leads to formation of an acyl imidazole (Scheme 1). Analogs can be prepared by reacting an acyl imidazole with an amine (3).

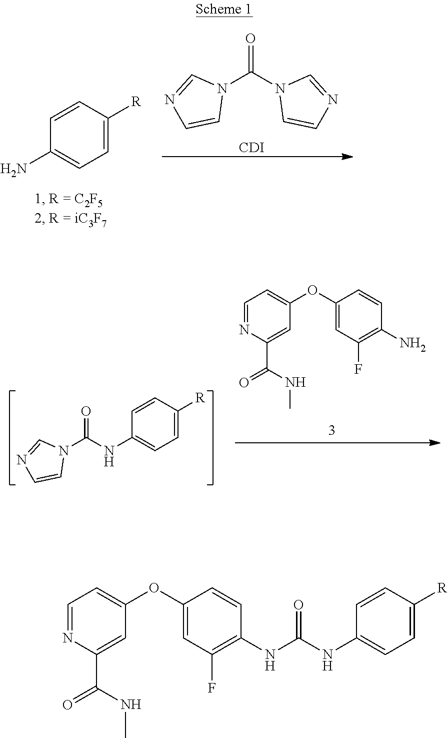

Other methods of preparing compounds of Formula (I) are described in the Examples below.

The compounds of Formula (I) can be isolated and purified in a known manner, for example, by subjecting the residue after distillation of the solvent to partition, extraction, re-precipitation, re-crystallization, or another purification method or combination of purification methods.

Methods of Treating Tumors and Cancers

One aspect of the present disclosure relates to a method of treating a tumor having a dysregulated Wnt signaling pathway. This method involves contacting a tumor having a dysregulated Wnt signaling pathway with a compound of Formula (I) under conditions effective to treat the tumor.

Another aspect of the present disclosure relates to a method of a treating cancer having a dysregulated Wnt signaling pathway in a subject in need thereof. This method involves administering to the subject a compound of Formula (I) under conditions effective to treat the subject for the cancer.

The term “tumor” or “neoplasm” refers to an abnormal mass of tissue that forms when cells grow and divide more than they should or do not die when they should. Tumors may be benign (not cancer) or malignant (cancer). Benign tumors may grow large but do not spread into, or invade, nearby tissues or other parts of the body. Malignant tumors can spread into, or invade, nearby tissues. They can also spread to other parts of the body through the blood and lymph systems.

The term “cancer” or “malignancy” refers to a diseases in which abnormal cells divide without control and can invade nearby tissues. Cancer cells can also spread to other parts of the body through the blood and lymph systems. There are several main types of cancer. Carcinoma is a cancer that begins in the skin or in tissues that line or cover internal organs. Sarcoma is a cancer that begins in bone, cartilage, fat, muscle, blood vessels, or other connective or supportive tissue. Leukemia is a cancer that begins in blood-forming tissue, such as the bone marrow, and causes too many abnormal blood cells to be made. Lymphoma and multiple myeloma are cancers that begin in the cells of the immune system. Central nervous system cancers are cancers that begin in the tissues of the brain and spinal cord.

The term “treating” means amelioration or relief from the symptoms and/or effects associated with the diseases or disorders described herein. Thus, “treating a tumor” or “treating a cancer” encompasses: (1) preventing, delaying, or reducing the incidence and/or likelihood of the appearance of at least one clinical or sub-clinical symptom of the tumor or cancer in a subject that may be afflicted with or predisposed to the tumor or cancer, but does not yet experience or display clinical or subclinical symptoms of the tumor or cancer; or (2) inhibiting the tumor or cancer, i.e., arresting, reducing or delaying the development of the tumor or cancer or a relapse thereof or at least one clinical or sub-clinical symptom thereof; or (3) relieving the tumor or cancer, i.e., causing regression of the tumor or cancer or at least one of its clinical or sub-clinical symptoms. The benefit to a subject to be treated is either statistically significant or at least perceptible to the patient or to the physician.

As noted above, aberrant Wtt signaling is associated with tumors and cancer. Accordingly, the dysregulated Wnt signaling pathway may comprise a mutation in one or more genes selected from the group consisting of CTNNB1, APC, AXIN1, AXIN2, GSK3B, LGR5, RN-F43, ZNRF3, LRP6, FBXW7, and TCF7L2. Table 1 below identifies genes involved in the Wnt signaling pathway by their gene name, Gene TD No., and NCBI reference transcript accession number.

TABLE 1
Exemplary Wnt Signaling Pathway Genes and Transcript Variants

| | | | Reference Transcript |
|---|---|---|---|
| Gene | Gene ID No.* | Transcript Variant | Accession Nos.* |
| Homo sapiens β- | 1499 | transcript variant 1 | NM_001904.4 |
| catenin (CTNNB1) | | transcript variant 2 | NM_001098209.2 |
| | | transcript variant 3 | NM_001098210.2 |
| | | transcript variant 4 | NM_001330729.2 |
| | | transcript variant X5 | XM_006712983.2 |
| | | transcript variant X8 | XM_006712985.1 |
| | | transcript variant X3 | XM_017005738.1 |
| | | transcript variant X1 | XM_024453356.1 |
| | | transcript variant X2 | XM_024453357.1 |
| | | transcript variant X4 | XM_024453358.1 |
| | | transcript variant X6 | XM_024453359.1 |
| | | transcript variant X7 | XM_024453360.1 |
| Homo sapiens | 324 | transcript variant 3 | NM_000038.6 |
| adenomatous | | transcript variant 2 | NM_001127510.3 |
| polyposis coli (APC) | | transcript variant 1 | NM_001127511.3 |
| | | transcript variant 4 | NM_001354895.2 |
| | | transcript variant 5 | NM_001354896.2 |
| | | transcript variant 6 | NM_001354897.2 |
| | | transcript variant 7 | NM_001354898.2 |
| | | transcript variant 8 | NM_001354899.2 |
| | | transcript variant 9 | NM_001354900.2 |
| | | transcript variant 10 | NM_001354901.2 |
| | | transcript variant 11 | NM_001354902.2 |
| | | transcript variant 12 | NM_001354903.2 |
| | | transcript variant 13 | NM_001354904.2 |
| | | transcript variant 14 | NM_001354905.2 |
| | | transcript variant 15 | NM_001354906.2 |
| Homo sapiens Axin 1 | 8312 | transcript variant 1 | NM_003502.4 |
| (AXIN1) | | transcript variant 2 | NM_181050.3 |
| | | transcript variant 3 | NR_134879.2 |
| | | transcript variant X1 | XM_011522682.2 |
| | | transcript variant X3 | XM_011522683.2 |
| | | transcript variant X5 | XM_011522684.2 |
| | | transcript variant X7 | XM_011522686.1 |
| | | transcript variant X6 | XM_017023745.2 |
| | | transcript variant X8 | XM_017023746.1 |
| | | transcript variant X9 | XM_017023747.1 |
| | | transcript variant X10 | XM_017023748.1 |
| | | transcript variant X11 | XR_001751996.1 |
| Homo sapiens Axin 2 | 8313 | transcript variant 1 | NM_004655.4 |
| (AXIN2) | | transcript variant 2 | NM_001363813.1 |
| | | transcript variant X2 | XM_011525319.2 |
| | | transcript variant X1 | XM_011525320.1 |
| | | transcript variant X3 | XM_011525321.2 |
| | | transcript variant X4 | XM_017025192.1 |
| | | transcript variant X5 | XM_017025193.1 |
| Homo sapiens | 2932 | transcript variant 2 | NM_001146156.2 |
| glycogen synthase | | transcript variant 1 | NM_002093.4 |
| kinase 3 beta | | transcript variant 3 | NM_001354596.2 |
| (GSK3B) | | transcript variant X2 | XM_006713610.3 |
| | | transcript variant X1 | XR_002959518.1 |
| Homo sapiens leucine | 8549 | transcript variant 1 | NM_003667.4 |
| rich repeat containing | | transcript variant 2 | NM_001277226.2 |
| G protein-coupled | | transcript variant 3 | NM_001277227.2 |
| receptor 5 (LGR5) | | transcript variant 4 | NR_110596.2 |
| Homo sapiens ring | 54894 | transcript variant 1 | NM_017763.6 |
| finger protein 43 | | transcript variant 2 | NM_001305544.2 |
| (RNF43) | | transcript variant 3 | NM_001305545.1 |
| | | transcript variant X1 | XM_011524955.3 |
| | | transcript variant X3 | XM_011524956.3 |
| | | transcript variant X2 | XM_017024800.2 |
| Homo sapiens zinc | 84133 | transcript variant 1 | NM_001206998.2 |
| and ring finger 3 | | transcript variant 2 | NM_032173.4 |
| (ZNR_F3) | | transcript variant X2 | XM_011530435.2 |
| | | transcript variant X3 | XM_011530436.3 |
| | | transcript variant X4 | XM_011530437.1 |
| | | transcript variant X5 | XM_011530438.2 |
| | | transcript variant X1 | XM_017028990.1 |
| | | transcript variant X6 | XM_024452286.1 |
| Homo sapiens LDL | 4040 | mRNA | NM_002336.3 |
| Receptor Related | | transcript variant X2 | XM_006719078.4 |
| Protein 6 (LRP6) | | transcript variant X4 | XM_011520671.3 |
| | | transcript variant X3 | XR_429035.3 |
| | | transcript variant X1 | XR_002957325.1 |
| Homo sapiens F-box | 55294 | transcript variant 5 | NM_001349798.2 |
| and WD Repeat | | transcript variant 2 | NM_018315.5 |
| Domain Containing 7 | | transcript variant 1 | NM_033632.3 |
| (FBXW7) | | transcript variant 3 | NM_001013415.2 |
| | | transcript variant 4 | NM_001257069.1 |
| | | transcript variant X5 | XM_011532084.2 |
| | | transcript variant X6 | XM_011532085.2 |
| | | transcript variant X7 | XM_011532086.2 |
| | | transcript variant X9 | XM_011532087.2 |
| | | transcript variant X10 | XM_011532088.2 |
| | | transcript variant X1 | XM_024454121.1 |
| | | transcript variant X2 | XM_024454122.1 |
| | | transcript variant X3 | XM_024454123.1 |
| | | transcript variant X4 | XM_024454124.1 |
| | | transcript variant X8 | XM_024454125.1 |
| | | transcript variant X11 | XM_024454126.1 |
| Homo sapiens | 6934 | transcript variant 1 | NM_001146274.2 |
| Transcription Factor 7 | | transcript variant 2 | NM_030756.5 |
| Like 2 (TCF7L2) | | transcript variant 3 | NM_001146283.2 |
| | | transcript variant 4 | NM_001146284.2 |
| | | transcript variant 5 | NM_001146285.2 |
| | | transcript variant 6 | NM_001146286.2 |
| | | transcript variant 7 | NM_001198525.2 |
| | | transcript variant 8 | NM_001198526.2 |
| | | transcript variant 9 | NM_001198527.2 |
| | | transcript variant 10 | NM_001198528.2 |
| | | transcript variant 11 | NM_001198529.2 |
| | | transcript variant 12 | NM_001198530.2 |
| | | transcript variant 13 | NM_001198531.2 |
| | | transcript variant 14 | NM_001349870.2 |
| | | transcript variant 15 | NM_001349871.1 |
| | | transcript variant 16 | NM_001363501.2 |
| | | transcript variant 17 | NM_001367943.1 |

*Each of which is hereby incorporated by reference in its entirety.

In some embodiments, the dysregulated signaling pathway comprises a mutation in CTNNB1. For example, the Wnt signaling pathway mutation may comprise a mutation in the β-catenin protein encoded by CTNNB1, the sequence of which (SEQ TD NO: 1) is as follows: MATQADLMELDMAMEPDRKAAVSHWQQQSYLDSGIHSGATTTAPSLSGKGNPEEEDVDTSQVLY EWEQGFSQSFTQEQVADIDGQYAMTRAQRVRAAMFPETLDEGMQIPSTQFDAAHPTNVQRLAEP SQMLKHAVVNLINYQDDAELATPAIPELTKLLNDEDQVVVNKAAVMVHQLSKKEASRHAIMRSP QMVSAIVRTMQNTNDVETARCTAGTLHNLSHHREGLLAIFKSGGIPALVKMLGSPVDSVLFYAI TTLHNLLLHQEGAKMAVRLAGGLQKMVALLNKTNVKFLAITTDCLQILAYGNQESKLIILASGG PQALVNIMRTYTYEKLLWTTSRVLKVLSVCSSNKPAIVEAGGMQALGLHLTDPSQRLVQNCLWT LRNLSDAATKQEGMEGLLGTLVQLLGSDDINVVTCAAGILSNLTCNNYKNKMMVCQVGGIEALV RTVLRAGDREDITEPAICALRHLTSRHQEAEMAQNAVRLHYGLPVVVKLLHPPSHWPLIKATVG LIRNLALCPANHAPLREQGAIPRLVQLLVRAHQDTQRRTSMGGTQQQFVEGVRMEEIVEGCTGA LHILARDVHNRIVIRGLNTIPLFVQLLYSPIENIQRVAAGVLCELAQDKEAAEAIEAEGATAPL TELLHSRNEGVATYAAAVLFRMSEDKPQDYKKRLSVELTSSLFRTEPMAWNETADLGLDIGAQG EPLGYRQDDPSYRSFHSGGYGQDALGMDPMMEHEMGGHHPGADYPVDGLPDLGHAQDLMDGLPP GDSNQLAWFDTDL. See UniProtKB entry P35222 (CTNB1_HUMAN). In some embodiments, the tumor encodes β-catenin comprising an N-terminal phosphodegron mutation or exon 3 indels.

As described herein, β-catenin comprises three domains: an N-terminal domain (˜130 aa), a central domain (residue 141-664) comprising 12 Armadillo (Arm) repeats, and a C-terminal domain (˜100 aa) (see, e.g., Kim & Jeong, “Mutation Hotspots in the β-Catenin Gene: Lessons from the Human Cancer Genome Databases,” Mol. Cells 42(1):8-16 (2019), which is hereby incorporated by reference in its entirety). The N-terminal domain is encoded by exon 3 (amino acid residues 5-80) of CTNNB1 and contains the phosphodegron sequence that binds to the F-box containing E3 ubiquitin ligase protein β-TrCP, leading to β-catenin ubiquitination by the SCF^{β-TrCP} cullin-RING ligase and subsequent degradation by the 26S proteasome (Simonetta et al., “Prospective Discovery of Small Molecule Enhancers of an E3 Ligase-Substrate Interaction,” Nature Comm. 10:1402 (2019), which is hereby incorporated by reference in its entirety). Mutations in the phosphodegron sequence of β-catenin impair the ability of β-catenin to effectively bind to β-TrCP leading to its stabilization and thereby facilitating an enhanced oncogenic transcriptional program. Thus, in some embodiments of the methods disclosed herein, the tumor and/or cancer encodes a β-catenin comprising an N-terminal phosphodegron mutation.

The β-catenin N-terminal phosphodegron mutation may be selected from the group consisting of D32G/N/Y/V/H/A, S33C/F/Y/P/A/T/L, G34R/E/V, I35S, 36P, S37F/C/A/P/Y, T41A/I/N, and S45F/P/Y/C/del or deletions in exon 3 and 4. As described supra, these mutations dysregulate the Wnt signaling pathway by disturbing phosphorylation-dependent ubiquitination of β-catenin. S45 is a priming-phosphorylation site for Casein Kinase I alpha (CK1α). S33, S37, and T41 are further phosphorylated by GSK3P.

In some embodiments, the β-catenin mutation occurs in a location of the β-catenin selected from the group consisting of D32, S33, G34, 135, H36, S37, T41, S45, K335, N387, W383, and combinations thereof (see, e.g., Kim & Jeong, “Mutation Hotspots in the β-Catenin Gene: Lessons from the Human Cancer Genome Databases,” Mol. Cells 42(1):8-16 (2019), which is hereby incorporated by reference in its entirety). D32 and G34 are required for binding with β-TrCP, a component of ubiquitin E3 ligase for phosphorylated β-catenin. K335, W383, and N387 are found in armadillo repeats 5 and 6 of β-catenin and are involved in binding to APC (Liu et al., “Oncogenic Mutations in Armadillo Repeats 5 and 6 of β-Catenin Reduce Binding to APC, Increasing Signaling and Transcription of Target Genes,” Gastroenterology 158(4):1029-1043 (2020), which is hereby incorporated by reference in its entirety). The mutation may be a substitution mutation, splice junction mutation, or a termination codon mutation.

Additional exemplary Wnt signaling pathway mutations in β-catenin may be selected from the group consisting of S23R, A25-33, D32G/N/Y/V/H/A, S33C/F/Y/P/A/T/L, G34R/E/V, I35S, H36P, S37F/C/A/P/Y, T41A/I/N, S45F/P/Y/C, ΔS45, L388P, A558-781, M688V, R710C, large deletions in CTNNB1, which encompass exon 3 and part of exon 4 or small insertions in Exon 3 and combinations thereof.

In some embodiments, the dysregulated signaling pathway comprises a mutation in APC. For example, the Wnt signaling pathway mutation may comprise a mutation in the adenomatous polyposis coli (APC) protein encoded by APC, the sequence of which (SEQ ID NO:2) is as follows: MAAASYDQLLKQVEALKMENSNLRQELEDNSNHLTKLETEASNMKEVLKQLQGSIEDEAMASSG QIDLLERLKELNLDSSNFPGVKLRSKMSLRSYGSREGSVSSRSGECSPVPMGSFPRRGFVNGSR ESTGYLEELEKERSLLLADLDKEEKEKDWYYAQLQNLTKRIDSLPLTENFSLQTDMTRRQLEYE ARQIRVAMEEQLGTCQDMEKPAQRRIARIQQIEKDILRIRQLLQSQATEAERSSQNKHETGSHD AERQNEGQGVGEINMATSGNGQGSTTRMDHETASVLSSSSTHSAPRRLTSHLGTKVEMVYSLLS MLGTHDKDDMSRTLLAMSSSQDSCISMRQSGCLPLLIQLLHGNDKDSVLLGNSRGSKEAPARAS AALHNIIHSQPDDKRGRREIRVLHLLEQIRAYCETCWEWQEAHEPGMDQDKNPMPAPVEHQICP AVCVLMKLSFDEEHRHAMNELGGLQAIAELLQVDCEMYGLTNDHYSITLRRYAGMALTNLTFGD VANKATLCSMKGCMRALVAQLKSESEDLQQVIASVLRNLSWRADVNSKKTLREVGSVKALMECA LEVKKESTLKSVLSALWNLSAHCTENKADICAVDGALAFLVGTLTYRSQTNTLAIIESGGGILR NVSSLIATNEDHRQILRENNCLQTLLQHLKSHSLTIVSNACGTLWNLSARNPKDQEALWDMGAV SMLKNLIHSKHKMIAMGSAAALRNLMANRPAKYKDANIMSPGSSLPSLHVRKQKALEAELDAQH LSETFDNIDNLSPKASHRSKQRHKQSLYGDYVFDTNRHDDNRSDNFNTGNMTVLSPYLNTTVLP SSSSSRGSLDSSRSEKDRSLERERGIGLGNYHPATENPGTSSKRGLQISTTAAQIAKVMEEVSA IHTSQEDRSSGSTTELHCVTDERNALRRSSAAHTHSNTYNFTKSENSNRTCSMPYAKLEYKRSS NDSLNSVSSSDGYGKRGQMKPSIESYSEDDESKFCSYGQYPADLAHKIHSANHMDDNDGELDTP INYSLKYSDEQLNSGRQSPSQNERWARPKHIIEDEIKQSEQRQSRNQSTTYPVYTESTDDKHLK FQPHFGQQECVSPYRSRGANGSETNRVGSNHGINQNVSQSLCQEDDYEDDKPTNYSERYSEEEQ HEEEERPTNYSIKYNEEKRHVDQPIDYSLKYATDIPSSQKQSFSFSKSSSGQSSKTEHMSSSSE NTSTPSSNAKRQNQLHPSSAQSRSGQPQKAATCKVSSINQETIQTYCVEDTPICFSRCSSLSSL SSAEDEIGCNQTTQEADSANTLQIAEIKEKIGTRSAEDPVSEVPAVSQHPRTKSSRLQGSSLSS ESARHKAVEFSSGAKSPSKSGAQTPKSPPEHYVQETPLMFSRCTSVSSLDSFESRSIASSVQSE PCSGMVSGIISPSDLPDSPGQTMPPSRSKTPPPPPQTAQTKREVPKNKAPTAEKRESGPKQAAV NAAVQRVQVLPDADTLLHFATESTPDGFSCSSSLSALSLDEPFIQKDVELRIMPPVQENDNGNE TESEQPKESNENQEKEAEKTIDSEKDLLDDSDDDDIEILEECIISAMPTKSSRKAKKPAQTASK LPPPVARKPSQLPVYKLLPSQNRLQPQKHVSFTPGDDMPRVYCVEGTPINFSTATSLSDLTIES PPNELAAGEGVRGGAQSGEFEKRDTIPTEGRSTDEAQGGKTSSVTIPELDDNKAEEGDILAECI NSAMPKGKSHKPFRVKKIMDQVQQASASSSAPNKNQLDGKKKKPTSPVKPIPQNTEYRTRVRKN ADSKNNLNAERVFSDNKDSKKQNLKNNSKVFNDKLPNNEDRVRGSFAFDSPHHYTPIEGTPYCF SRNDSLSSLDFDDDDVDLSREKAELRKAKENKESEAKVTSHTELTSNQQSANKTQAIAKQPINR GQPKPILQKQSTFPQSSKDIPDRGAATDEKLQNFAIENTPVCFSHNSSLSSLSDIDQENNNKEN EPIKETEPPDSQGEPSKPQASGYAPKSFHVEDTPVCFSRNSSLSSLSIDSEDDLLQECISSAMP KKKKPSRLKGDNEKHSPRNMGGILGEDLTLDLKDIQRPDSEHGLSPDSENFDWKAIQEGANSIV SSLHQAAAAACLSRQASSDSDSILSLKSGISLGSPFHLTPDQEEKPFTSNKGPRILKPGEKSTL ETKKIESESKGIKGGKKVYKSLITGKVRSNSEISGQMKQPLQANMPSISRGRTMIHIPGVRNSS SSTSPVSKKGPPLKTPASKSPSEGQTATTSPRGAKPSVKSELSPVARQTSQIGGSSKAPSRSGS RDSTPSRPAQQPLSRPIQSPGRNSISPGRNGISPPNKLSQLPRTSSPSTASTKSSGSGKMSYTS PGRQMSQQNLTKQTGLSKNASSIPRSESASKGLNQMNNGNGANKKVELSSRMSSTKSSGSESDRS ERPVLVRQSTFIKEAPSPTLRRKLEESASFESLSPSSRPASPTRSQAQTPVLSPSLPDMSLSTH SSVQAGGWRKLPPNLSPTIEYNDGRPAKRHDIARSHSESPSRLPINRSGTWKREHSKHSSSLPR VSTWRRTGSSSSILSASSESSEKAKSEDEKHVNSISGTKQSKENQVSAKGTWRKIKENEFSPTN STSQTVSSGATNGAESKTLIYQMAPAVSKTEDVWVRIEDCPINNPRSGRSPTGNTPPVIDSVSE KANPNIKDSKDNQAKQNVGNGSVPMRTVGLENRLNSFIQVDAPDQKGTEIKPGQNNPVPVSETN ESSIVERTPFSSSSSSKHSSPSGTVAARVTPFNYNPSPRKSSADSTSARPSQIPTPVNNNTKKR DSKTDSTESSGTQSPKRHSGSYLVTSV. See UniProtKB entry P25054 (APC_HUMAN). Exemplary Wnt signaling pathway mutations in APC may be selected from the group consisting of R99W, S171I, R414C, S722G, S784T, E911G, P1176L, A1184P, T1292M, T1313A, R1348W, S2621C, L2839F, A1296V, V1472I, S1495G, K516E, R549E, T2841L, V2843Q, and combinations thereof.

In some embodiments, the dysregulated signaling pathway comprises a mutation in AXIN1. For example, the Wnt signaling pathway mutation may comprise a mutation in the Axin1 protein encoded by AXIN1, the sequence of which (SEQ ID NO:3) is as follows: MNIQEQGFPLDLGASFTEDAPRPPVPGEEGELVSTDPRPASYSFCSGKGVGIKGETSTATPRRS DLDLGYEPEGSASPTPPYLKWAESLHSLLDDQDGISLFRTFLKQEGCADLLDFWFACTGFRKLE PCDSNEEKRLKLARAIYRKYILDNNGIVSRQTKPATKSFIKGCIMKQLIDPAMFDQAQTEIQAT MEENTYPSFLKSDIYLEYTRTGSESPKVCSDQSSGSGTGKGISGYLPTLNEDEEWKCDQDMDED DGRDAAPPGRLPQKLLLETAAPRVSSSRRYSEGREFRYGSWREPVNPYYVNAGYALAPATSAND SEQQSLSSDADTLSLTDSSVDGIPPYRIRKQHRREMQESVQVNGRVPLPHIPRTYRVPKEVRVE PQKFAEELIHRLEAVQRTREAEEKLEERLKRVRMEEEEGEDGDPSSGPPGPCHKLPPAPAWHHFP PRCVDMGCAGLRDAHEENPESILDEHVQRVLRTPGRQSPGPGHRSPDSGHVAKMPVALGGAASG HGKHVPKSGAKLDAAGLHHHRHVHHHVHHSTARPKEQVEAEATRRAQSSFAWGLEPHSHGARSR GYSESVGAAPNASDGLAHSGKVGVACKRNAKKAESGKSASTEVPGASEDAEKNQKIMQWIIEGE KEISRHRRTGHGSSGTRKPQPHENSRPLSLEHPWAGPQLRTSVQPSHLFIQDPTMPPHPAPNPL TQLEEARRRLEEEEKPASRAPSKQRYVQEVMRRGRACVRPACAPVLHVVPAVSDMELSETETRS QRKVGGGSAQPCDSIVVAYYFCGEPIPYRTLVRGRAVTLGQFKELLTKKGSYRYYFKKVSDEFD CGVVFEEVREDEAVLPVFEEKIIGKVEKVD. See UniProtKB entry 015169 (AXIN1_HUMAN). Exemplary Wnt signaling pathway mutations in Axin1 may be selected from the group consisting of L106R, P345L, G425S, G650S, R799Q, R146*, and combinations thereof.

In some embodiments, the dysregulated signaling pathway comprises a mutation in AXIN2. For example, the Wnt signaling pathway mutation may comprise a mutation in the Axin2 protein encoded by AXIN2, the sequence of which (SEQ ID NO:4) is as follows: MSSAMLVTCLPDPSSSFREDAPRPPVPGEEGETPPCQPGVGKGQVTKPMSVSSNTRRNEDGLGE PEGPASPDSPLTRWTKSLHSLLGDQDGAYLFRTFLEREKCVDTLDFWFACNGFRQMNLKDTKTL RVAKAIYKRYIENNSIVSKQLKPATKTYIRDGIKKQQIDSIMFDQAQTEIQSVMEENAYQMFLT SDIYLEYVRSGGENTAYMSNGGLGSLKVVCGYLPTLNEEEEWTCADFKCKLSPTVVGLSSKTLR ATASVRSTETVDSGYRSFKRSDPVNPYHIGSGYVFAPATSANDSEISSDALTDDSMSMTDSSVD GIPPYRVGSKKQLQREMHRSVKANGQVSLPHFPRTHRLPKEMTPVEPATFAAELISRLEKLKLE LESRHSLEERLQQIREDEEREGSELTLNSREGAPTQHPLSLLPSGSYEEDPQTILDDHLSRVLK TPGCQSPGVGRYSPRSRSPDHHHHHHSQYHSLLPPGGKLPPAAASPGACPLLGGKGFVTKQTTK HVHHHYIHHHAVPKTKEEIEAEATQRVHCFCPGGSEYYCYSKCKSHSKAPETMPSEQFGGSRGS TLPKRNGKGTEPGLALPAREGGAPGGAGALQLPREEGDRSQDVWQWMLESERQSKPKPHSAQST KKAYPLESARSSPGEPASRHHLWGGNSGHPRTTPPAHLFTQDPAMPPLTPPNTLAQLEEACRRL AEVSKPPKQRCCVASQQRDRNHSATVQTGATPFSNPSLAPEDHKEPKKLAGVHALQASELVVTY FFCGEEIPYRRMLKAQSLTLGHFKEQLSKKGNYRYYFKKASDEFACGAVFEEIWEDETVLPMYE GRILGKVERID. See UniProtKB entry Q9Y2T1 (AXIN2_HUMAN). Exemplary Wnt signaling pathway mutations in Axin2 may be selected from the group consisting of A578fs, A643fs, Q631*, Q696*, E633*, E698*, E612fs, M620fs, M685fs, and combinations thereof.

In some embodiments, the dysregulated signaling pathway comprises a mutation in glycogen synthase kinase 3 beta. For example, the Wnt signaling pathway mutation may comprise a mutation in the glycogen synthase kinase 3 beta protein encoded by GSK3B, the sequence of which (SEQ ID NO:5) is as follows: MSGRPRTTSFAESCKPVQQPSAFGSMKVSRDKDGSKVTTVVATPGQGPDRPQEVSYTDTKVIGN GSFGVVYQAKLCDSGELVAIKKVLQDKRFKNRELQIMRKLDHCNIVRLRYFFYSSGEKKDEVYL NLVLDYVPETVYRVARHYSRAKQTLPVIYVKLYMYQLFRSLAYIHSFGICHRDIKPQNLLLDPD TAVLKLCDFGSAKQLVRGEPNVSYICSRYYPAPELIFGATDYTSSIDVWSAGCVLAELLLGQPI FPGDSGVDQLVEIIKVLGTPTREQIREMNPNYTEFKFPQIKAHPWTKVFRPRTPPEAIALCSRL LEYTPTARLTPLEACAHSFFDELRDPNVKLPNGRDTPALFNFTTQELSSNPPLATILIPPHARI QAAASTPTNATAASDANTGDRGQTNNAASASASNST. See UniProtKB entry P49841 (GSK3B_HUMAN).

In some embodiments, the dysregulated signaling pathway comprises a mutation in leucine rich repeat containing G protein-coupled receptor 5. For example, the Wnt signaling pathway mutation may comprise a mutation in the leucine rich repeat containing G protein-coupled receptor 5 protein encoded by LGR5, the sequence of which (SEQ ID NO:6) is as follows: MDTSRLGVLLSLPVLLQLATGGSSPRSGVLLRGCPTHCHCEPDGRMLLRVDCSDLGLSELPSNL SVFTSYLDLSMNNISQLLPNPLPSLRFLEELRLAGNALTYIPKGAFTGLYSLKVLMLQNNQLRH VPTEALQNLRSLQSLRLDANHISYVPPSCFSGLHSLRHLWLDDNALTEIPVQAFRSLSALQAMT LALNKIHHIPDYAFGNLSSLVVLHLHNNRIHSLGKKCFDGLHSLETLDLNYNNLDEFPTAIRTL SNLKELGFHSNNIRSIPEKAFVGNPSLITIHFYDNPIQFVGRSAFQHLPELRTLTLNGASQITE FPDLTGTANLESLTLTGAQISSLPQTVCNQLPNLQVLDLSYNLLEDLPSFSVCQKLQKIDLRHN EIYEIKVDTFQQLLSLRSLNLAWNKIAIIHPNAFSTLPSLIKLDLSSNLLSSFPITGLHGLTHL KLTGNHALQSLISSENFPELKVIEMPYAYQCCAFGVCENAYKISNQWNKGDNSSMDDLHKKDAG MFQAQDERDLEDFLLDFEEDLKALHSVQCSPSPGPFKPCEHLLDGWLIRIGVWTIAVLALTCNA LVTSTVFRSPLYISPIKLLIGVIAAVNMLTGVSSAVLAGVDAFTFGSFARHGAWWENGVGCHVI GFLSIFASESSVFLLTLAALERGFSVKYSAKFETKAPFSSLKVIILLCALLALTMAAVPLLGGS KYGASPLCLPLPFGEPSTMGYMVALILLNSLCFLMMTIAYTKLYCNLDKGDLENIWDCSMVKHI ALLLFTNCILNCPVAFLSFSSLINLTFISPEVIKFILLVVVPLPACLNPLLYILFNPHFKEDLV SLRKQTYVWTRSKHPSLMSINSDDVEKQSCDSTQALVTFTSSSITYDLPPSSVPSPAYPVTESC HLSSVAFVPCL. See UniProtKB entry 075473 (LGR5_HUMAN). Exemplary Wnt signaling pathway mutations in leucine rich repeat containing G protein-coupled receptor 5 may be selected from the group consisting of D146F, D170F, A190D, and combinations thereof.

In some embodiments, the dysregulated signaling pathway comprises a mutation in ring finger protein 43. For example, the Wnt signaling pathway mutation may comprise a mutation in the ring finger protein 43 protein encoded by RNF43, the sequence of which (SEQ ID NO:7) is as follows: MSGGHQLQLAALWPWLLMATLQAGFGRTGLVLAAAVESERSAEQKAIIRVIPLKMDPTGKLNLT LEGVFAGVAEITPAEGKLMQSHPLYLCNASDDDNLEPGFISIVKLESPRRAPRPCLSLASKARM AGERGASAVLFDITEDRAAAEQLQQPLGLTWPVVLIWGNDAEKLMEFVYKNQKAHVRIELKEPP AWPDYDVWILMTVVGTIFVIILASVLRIRCRPRHSRPDPLQQRTAWAISQLATRRYQASCRQAR GEWPDSGSSCSSAPVCAICLEEFSEGQELRVISCLHEFHRNCVDPWLHQHRTCPLCMFNITEGD SFSQSLGPSRSYQEPGRRLHLIRQHPGHAHYHLPAAYLLGPSRSAVARPPRPGPFLPSQEPGMG PRHHRFPRAAHPPAPGEQQRLAGAQHPYAQGWGLSHLQSTSQHPAACPVPLRRARPPDSSGSGE SYCTERSGYLADGPASDSSSGPCHGSSSDSVVNCTDISLQGVHGSSSTFCSSLSSDFDPLVYCS PKGDPQRVDMQPSVTSRPRSLDSVVPTGETQVSSHVHYHRHRHHHYKKRFQWHGRKPGPETGVP QSRPPIPRTQPQPEPPSPDQQVTRSNSAAPSGRLSNPQCPRALPEPAPGPVDASSICPSTSSLF NLQKSSLSARHPQRKRRGGPSEPTPGSRPQDATVHPACQIFPHYTPSVAYPWSPEAHPLICGPP GLDKRLLPETPGPCYSNSQPVWLCLTPRQPLEPHPPGEGPSEWSSDTAEGRPCPYPHCQVLSAQ PGSEEELEELCEQAV. See UniProtKB entry Q68DV7(RNF43 HUMAN). Exemplary Wnt signaling pathway mutations in ring finger protein 43 protein may be selected from the group consisting of C290S, H292S, H295S, C298S, and combinations thereof.

In some embodiments, the dysregulated signaling pathway comprises a mutation in zinc and ring finger 3. For example, the Wnt signaling pathway mutation may comprise a mutation in the zinc and ring finger 3 protein encoded by ZNRF3, the sequence of which (SEQ ID NO:8) is as follows: MRPRSGGRPGATGRRRRRLRRRPRGLRCSRLPPPPPLPLLLGLLLAAAGPGAARAKETAFVEVV LFESSPSGDYTTYTTGLTGRFSRAGATLSAEGEIVQMHPLGLCNNNDEEDLYEYGWVGVVKLEQ PELDPKPCLTVLGKAKRAVQRGATAVIFDVSENPEAIDQLNQGSEDPLKRPVVYVKGADAIKLM NIVNKQKVARARIQHRPPRQPTEYFDMGIFLAFFVVVSLVCLILLVKIKLKQRRSQNSMNRLAV QALEKMETRKFNSKSKGRREGSCGALDTLSSSSTSDCAICLEKYIDGEELRVIPCTHRFHRKCV DPWLLQHHTCPHCRHNIIEQKGNPSAVCVETSNLSRGRQQRVTLPVHYPGRVHRTNAIPAYPTR TSMDSHGNPVTLLTMDRHGEQSLYSPQTPAYIRSYPPLHLDHSLAAHRCGLEHRAYSPAHPFRR PKLSGRSFSKAACFSQYETMYQHYYFQGLSYPEQEGQSPPSLAPRGPARAFPPSGSGSLLFPTV VHVAPPSHLESGSTSSFSCYHGHRSVCSGYLADCPGSDSSSSSSSGQCHCSSSDSVVDCTEVSN QGVYGSCSTFRSSLSSDYDPFIYRSRSPCRASEAGGSGSSGRGPALCFEGSPPPEELPAVHSHG AGRGEPWPGPASPSGDQVSTCSLEMNYSSNSSLEHRGPNSSTSEVGLEASPGAAPDLRRTWKGG HELPSCACCCEPQPSPAGPSAGAAGSSTLFLGPHLYEGSGPAGGEPQSGSSQGLYGLHPDHLPR TDGVKYEGLPCCFYEEKQVARGGGGGSGCYTEDYSVSVQYTLTEEPPPGCYPGARDLSQRIPII PEDVDCDLGLPSDCQGTHSLGSWGGTRGPDTPRPHRGLGATREEERALCCQAPALLRPGCPPEE AGAVRANFPSALQDTQESSTTATEAAGPRSHSADSSSPGA. See UniProtKB entry Q9ULT6 (ZNRF3_HUMAN).

In some embodiments, the dysregulated signaling pathway comprises a mutation in LDL Receptor Related Protein 6 protein. For example, the Wnt signaling pathway mutation may comprise a mutation in the LDL Receptor Related Protein 6 protein encoded by LRP6, the sequence of which (SEQ ID NO:9) is as follows: MGAVLRSLLACSFCVLLRAAPLLLYANRRDLRLVDATNGKENATIVVGGLEDAAAVDFVFSHGL IYWSDVSEEAIKRTEFNKTESVQNVVVSGLLSPDGLACDWLGEKLYWTDSETNRIEVSNLDGSL RKVLFWQELDQPRAIALDPSSGFMYWTDWGEVPKIERAGMDGSSRFIIINSEIYWPNGLTLDYE EQKLYWADAKLNFIHKSNLDGTNRQAVVKGSLPHPFALTLFEDILYWTDWSTHSILACNKYTGE GLREIHSDIFSPMDIHAFSQQRQPNATNPCGIDNGGCSHLCLMSPVKPFYQCACPTGVKLLENG KTCKDGATELLLLARRTDLRRISLDTPDFTDIVLQLEDIRHAIAIDYDPVEGYIYWTDDEVRAI RRSFIDGSGSQFVVTAQIAHPDGIAVDWVARNLYWTDTGTDRIEVTRLNGTMRKILISEDLEEP RAIVLDPMVGYMYWTDWGEIPKIERAALDGSDRVVLVNTSLGWPNGLALDYDEGKIYWGDAKTD KIEVMNTDGTGRRVLVEDKIPHIFGFTLLGDYVYWTDWQRRSIERVHKRSAEREVIIDQLPDLM GLKATNVHRVIGSNPCAEENGGCSHLCLYRPQGLRCACPIGFELISDMKTCIVPEAFLLFSRRA DIRRISLETNNNNVAIPLTGVKEASALDFDVTDNRIYWTDISLKTISPAFMNGSALEHVVEFGL DYPEGMAVDWLGKNLYWADTGTNRIEVSKLDGQHRQVLVWKDLDSPPALALDPAEGFMYWTEWG GKPKIDRAAMDGSERTTLVPNVGRANGLTIDYAKRRLYWTDLDTNLIESSNMLGLNREVIADDL PHPFGLTQYQDYIYWTDWSRRSIERANKTSGQNRTIIQGHLDYVMDILVFHSSRQSGWNECASS NGHCSHLCLAVPVGGFVCGCPAHYSLNADNRTCSAPTTFLLFSQKSAINRMVIDEQQSPDIILP IHSLRNVPAIDYDPLDKQLYWIDSRQNMIRKAQEDGSQGFTVVVSSVPSQNLEIQPYDLSIDIY SRYIYWTCEATNVINVTRLDGRSVGVVLKGEQDRPPAVVVNPEKGYMYFTNLQERSPKIERAAL DGTEREVLFFSGLSKPIALALDSRLGKLFWADSDLRRIESSDLSGANRIVLEDSNILQPVGLTV FENWLYWIDKQQQMIEKIDMTGREGRTKVQARIAQLSDIHAVKELNLQEYRQHPCAQDNGGCSH ICLVKGDGTTRCSCPMHLVLLQDELSCGEPPTCSPQQFTCFTGEIDCIPVAWRCDGFTECEDHS DELNCPVCSESQFQCASGQCIDGALRCNGDANCQDKSDEKNCEVLCLIDQFRCANGQCIGKHKK CDHNVDCSDKSDELDCYPTEEPAPQATNTVGSVIGVIVTIFVSGTVYFICQRMLCPRMKGDGET MTNDYVVHGPASVPLGYVPHPSSLSGSLPGMSRGKSMISSLSIMGGSSGPPYDRAHVTGASSSS SSSTKGTYFPAILNPPPSPATERSHYTMEFGYSSNSPSTHRSYSYRPYSYRHFAPPTTPCSTDV CDSDYAPSRRMTSVATAKGYTSDLNYDSEPVPPPPTPRSQYLSAEENYESCPPSPYTERSYSHH LYPPPPSPCTDSS. See UniProtKB entry 075581 (LRP6_HUMAN). Exemplary Wnt signaling pathway mutations in LDL Receptor Related Protein 6 protein may be selected from the group consisting of A19V, R473W, R611C, K1403R, and combinations thereof.

In some embodiments, the dysregulated signaling pathway comprises a mutation in F-box and WD repeat domain containing 7 protein. For example, the Wnt signaling pathway mutation may comprise a mutation in the F-box and WD repeat domain containing 7 protein encoded by FBXW7, the sequence of which (SEQ ID NO:10) is as follows: MNQELLSVGSKRRRTGGSLRGNPSSSQVDEEQMNRVVEEEQQQQLRQQEEEHTARNGEVVGVEP RPGGQNDSQQGQLEENNNRFISVDEDSSGNQEEQEEDEEHAGEQDEEDEEEEEMDQESDDFDQS DDSSREDEHTHTNSVTNSSSIVDLPVHQLSSPFYTKTTKMKRKLDHGSEVRSFSLGKKPCKVSE YTSTTGLVPCSATPTTFGDLRAANGQGQQRRRITSVQPPTGLQEWLKMFQSWSGPEKLLALDEL IDSCEPTQVKHMMQVIEPQFQRDFISLLPKELALYVLSFLEPKDLLQAAQTCRYWRILAEDNLL WREKCKEEGIDEPLHIKRRKVIKPGFIHSPWKSAYIRQHRIDTNWRRGELKSPKVLKGHDDHVI TCLQFCGNRIVSGSDDNTLKVWSAVTGKCLRTLVGHTGGVWSSQMRDNIIISGSTDRTLKVWNA ETGECIHTLYGHTSTVRCMHLHEKRVVSGSRDATLRVWDIETGQCLHVLMGHVAAVRCVQYDGR RVVSGAYDFMVKVWDPETETCLHTLQGHTNRVYSLQFDGIHVVSGSLDTSIRVWDVETGNCIHT LTGHQSLTSGMELKDNILVSGNADSTVKIWDIKTGQCLQTLQGPNKHQSAVTCLQFNKNFVITS SDDGTVKLWDLKTGEFIRNLVTLESGGSGGVVWRIPASNTKLVCAVGSRNGTEETKLLVLDFDV DMK. See UniProtKB entry Q969H0 (FBXW7_HUMAN).

In some embodiments, the dysregulated signaling pathway comprises a mutation in transcription factor 7 like 2 protein. For example, the Wnt signaling pathway mutation may comprise a mutation in the transcription factor 7 like 2 protein encoded by TCF7L2, the sequence of which (SEQ ID NO:11) is as follows: MPQLNGGGGDDLGANDELISFKDEGEQEEKSSENSSAERDLADVKSSLVNESETNQNSSSDSEA ERRPPPRSESFRDKSRESLEEAAKRQDGGLFKGPPYPGYPFIMIPDLTSPYLPNGSLSPTARTL HFQSGSTHYSAYKTIEHQIAVQYLQMKWPLLDVQAGSLQSRQALKDARSPSPAHIVSNKVPVVQ HPHHVHPLTPLITYSNEHFTPGNPPPHLPADVDPKTGIPRPPHPPDISPYYPLSPGTVGQIPHP LGWLVPQQGQPVYPITTGGFRHPYPTALTVNASMSRFPPHMVPPHHTLHTTGIPHPAIVTPTVK QESSQSDVGSLHSSKHQDSKKEEEKKKPHIKKPLNAFMLYMKEMRAKVVAECTLKESAAINQIL GRRWHALSREEQAKYYELARKERQLHMQLYPGWSARDNYGKKKKRKRDKQPGETNEHSECFLNP CLSLPPITDLSAPKKCRARFGLDQQNNWCGPCRRKKKCVRYIQGEGSCLSPPSSDGSLLDSPPP SPNLLGSPPRDAKSQTEQTQPLSLSLKPDPLAHLSMMPPPPALLLAEATHKASALCPNGALDLP PAALQPAAPSSSIAQPSTSSLHSHSSLAGTQPQPLSLVTKSLE. See UniProtKB entry Q9NQB0 (TF7L2_HUMAN).

In some embodiments of the methods according to the present disclosure, the tumor is associated with a colorectal cancer; a gastric cancer; an endometrial cancer; a lung cancer; a liver cancer; a hepatocellular carcinoma; a hepatocellular adenoma; a hepatoblastoma; a melanoma; a bladder carcinoma; a pilomatrixoma; an ovarian cancer; a medulloblastoma; an adenocortical carcinoma; a pancreatic cancer; a NSCLC; a liver adenoma; a LIAD; a hepatoblastoma; or a cancer of the uterus, pancreas, prostate, stomach, bladder, anus, or esophagus.

Hepatocellular carcinoma (HCC) is a type of adenocarcinoma and the most common type of liver tumor. HCC is associated with mutations in β-catenin (encoded by CTNNB1), which occur in ˜30% of cases and are of particular therapeutic interest (Llovet et al., “Hepatocellular Carcinoma,” Nat. Rev. Dis. Primers 7(1):6 (2021); Harding et al., “Prospective Genotyping of Hepatocellular Carcinoma: Clinical Implications of Next-Generation Sequencing for Matching Patients to Targeted and Immune Therapies,” Clin. Cancer Res. 25(7):2116-2126 (2018); Ally et al., “Comprehensive and Integrative Genomic Characterization of Hepatocellular Carcinoma,” Cell 169(7):1327-1341 (2017); and Hoshida et al., “Integrative Transcriptome Analysis Reveals Common Molecular Subclasses of Human Hepatocellular Carcinoma,” Cancer Res. 69(18):7385-7392 (2009), which are hereby incorporated by reference in its entirety) as well as APC (Huang et al., “β-Catenin Mutations are Frequent in Human Hepatocellular Carcinomas Associated with Hepatitis C Virus Infection,” Am. J. Pathol. 155(6):1795-1801 (1999), which is hereby incorporated by reference in its entirety).

β-catenin mutations have also been identified in a variety of human tumors including, e.g., colon cancer, endometrial carcinoma, ovarian cancer, medulloblastoma, prostate cancer, and bone and soft-tissue tumors, as well as skin tumors, thyroid carcinoma, and childhood hepatoblastoma (see, e.g., Huang et al., “β-Catenin Mutations are Frequent in Human Hepatocellular Carcinomas Associated with Hepatitis C Virus Infection,” Am. J. Pathol. 155(6):1795-1801 (1999), which is hereby incorporated by reference in its entirety).

Exemplary Wnt signaling pathway mutations associated with tumors and/or cancers are identified in Table 2 and Table 3 below.

TABLE 2
Exemplary Wnt Signaling Pathway Mutations*

| Cancer | β-catenin Mutation | APC Mutation |
|---|---|---|
| Colorectal cancer | S33Y, T41A, S45del, S45F | R2714C, Y159C, R1788C |
| Gastric cancer | D32N, G34E, G34V, S45C | R499*, R1114*, R302* |
| | | R2204*, E1576* |
| | | T1556Nfs*3 |
| Endometrial cancer | D32V, X561_splice, S37P, | P1233L, S678G, A2388V, |
| | D207G, S37C, S37P | T2514I, A2V |
| Lung cancer | S33L, S33F, T41A, S45F, | P865L, A2122dup, D2796G |
| | Y670 | |
| Liver cancer | G34V, S37C | |
| Hepatocellular carcinoma | D32G/N/Y/V/H/A, | |
| | S33C/F/Y/P/A/T/L, | |
| | G34R/E/V, 135S, H36P, | |
| | S37F/C/A/P/Y, T41A/I/N, | |
| | S45F/P/Y/C | |
| | DeltaExon3, DeltaExon3-4 | |
| Hepatoblastoma | Indels in Exon3 | |
| Melanoma | S33C, S45del | R2204* |
| Bladder carcinoma | | E1353* |
| | | R2204* |
| Pilomatrixoma (PTR) | G34E, S37C, S37F, T41I | |
| Ovarian cancer | S37C, T41I | |
| Medulloblastoma | | A1296V, V1472I, S1495G |
| Adenocortical carcinoma | S45F/P, G34R/E/V | S1465Wfs*3, R1858* |

*See, e.g., Kim & Jeong, “Mutation Hotspots in the β-Catenin Gene: Lessons from the Human Cancer Genome Databases,” Mol. Cells 42(1): 8-16 (2019), which is hereby incorporated by reference in its entirety.

TABLE 3
Exemplary Wnt Signaling Pathway Mutations*

| | | ZNRF3 | AXIN1 | AXIN2 |
|---|---|---|---|---|
| Cancer | RNF43 Mutation | Mutation | Mutation | Mutation |
| Colorectal | Frame shift | Promoter | T79Pfs*5 | G665Afs*24/ |
| cancer | G659Vfs*41, | silencing by DNA | P432Qfs*48 | N666Qfs*41, |
| | V299Gfs*143 | methylation | X372_splice | E405Gfs*56 |
| | H295Ifs*124 | | K641Rfs*64 | |
| | | | R22Dfs*62 | |
| Gastric cancer | H86R/Y, Frame | | | |
| | shift | | | |
| | G659Vfs*41, | | | |
| | D516Gfs*10 | | | |
| Endometrial | Frame shift | C330Y/ | R146Q | G665Afs*24 |
| cancer | G659Vfs*41, | C330_P331insR/ | R841 | X400_splice |
| | G417V, R228G | H332Tfs*7 | T79Pfs*5 | A599Pfs*90 |
| | | R417C | G265Efs*149 | Q175* |
| | | | V598Gfs*13 | E366* |
| | | | S339 = splice | R339H |
| | | | G508Vfs*197 | P65L |
| | | | A443Lfs*37 | |
| | | | V835Wfs*103 | |
| Lung cancer | | | | G665Afs*24/ |
| | | | | N666Qfs*41 |
| Adenocortical | | E339*, | | |
| carcinoma | | X339_splice | | |
| | | R307W | | |
| | | L191Qfs*19 | | |
| | | V394Sfs*4 | | |
| Hepatocellular | | P175Q, R246H | X340_splice | |
| carcinoma | | R149P | P494Rfs*211 | |
| | | | T555Qfs*34 | |
| | | | W635* | |
| | | | K607* | |
| | | | E397* | |
| | | | Q96* | |
| Pancreatic | R145*, Q22*, | | | |
| | Frame shift | | | |
| | V205Wfs*7, | | | |
| | A11Lfs*27 | | | |
| | L61Qfs*13 | | | |
| | V287Gfs*7 | | | |
| Melanoma | | | | |
| NSCLC | | | Promoter | |
| | | | silencing by | |
| | | | DNA | |
| | | | methylation | |

Suitable compounds of Formula (I) for use in the methods according to the present disclosure are described in more detail supra. In some embodiments of the compound of Formula (I), X is fluorine.

In some embodiments of the compound of formula (I), R is a phenyl substituted with C2F₅ or C₃F₇.

In some embodiments, the compound of Formula (I) has the chemical structure of

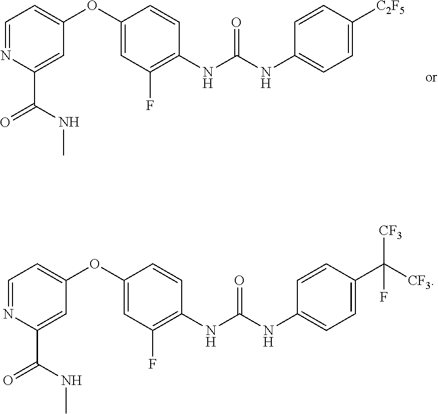

In some embodiments of the methods of the present disclosure, contacting a tumor having a dysregulated Wnt signaling pathway or treating a cancer having a dysregulated Wnt signaling pathway in a subject in need thereof with a compound of Formula (I) may further involve contacting a tumor having a dysregulated Wnt signaling pathway or administering to a subject an immune checkpoint inhibitor.

Immune checkpoint inhibitors are well known in the art. These drugs block different checkpoint proteins, including CTLA-4 (cytotoxic T lymphocyte associated protein 4), PD-1 (programmed cell death protein 1), PD-L1 (programmed death ligand 1), and PD-L2 (programmed death ligand 2). Thus, in some embodiments, the immune checkpoint inhibitor is selected from the group consisting of a CTLA-4 inhibitor, a PD-1 inhibitor, a PD-L1 inhibitor, and a PD-L2 inhibitor.

Programmed death protein 1 (PD-1) is an inhibitory receptor expressed by activated B cells, T cells, and natural killer (NK) cells, as well as some myeloid cells. PD-1 and its ligands, PD-L1 and PD-L2, control immune activity by causing a transient downregulation of T-cell function. Upregulated expression of PD-L1 on tumor and/or stromal cells in the tumor microenvironment enables engagement of PD-1 on activated T cells and functions to down-regulate T-cell activation, resulting in diminished antitumor T-cell responses.

In carrying out the methods described herein, the PD-1 pathway inhibitor may be an antibody. For example, the PD-1 pathway inhibitor may be an anti-PD-1 antibody. Suitable anti-PD-1 antibodies include, without limitation, nivolumab (OPDIVO©), pembrolizumab (KEYTRUDA©), cemiplimab (LIBTAYO©), pidilizumab (CT-011), REGN2810 (SAR-439684), spartalizumab (PDR001), camrelizumab (SHR1210), sintilimab (IBI308), tislelizumab (BGB-A317), toripalimab (JS 001), dostarlimab (TSR-042, WBP-285), INCMGA00012 (MGA012), AMP-224, AMP-514 (MEDI0680), and PF-06801591 (see, e.g., Liao et al., “A Review of Efficacy and Safety of Checkpoint Inhibitor for the Treatment of Acute Myeloid Leukemia,” Front. Pharmacol. 10: 609 (2019), which is hereby incorporated by reference in its entirety).

The fully human immunoglobulin G4 (IgG4) monoclonal antibody nivolumab (OPDIVO©, Bristol-Myers Squibb) and the humanized IgG4-κmonoclonal antibody pembrolizumab (KEYTRUDA©, Merck) target PD-1 to reverse the inhibitory signal and increase antitumor activity.

Similarly, monoclonal antibodies have been developed against PD-L1, including the humanized IgG1 agent atezolizumab (TECENTRIQ©, Genentech) and the fully human IgG1 agents avelumab (BAVENCIO©, EMD Serono/Pfizer), durvalumab (IMFINZI©, AstraZeneca), KN035, CK-301, AUNP12, CA-170, BMS-986189, MPDL3280A, and MEDI4736 (see, e.g., Powles et al., “MPDL3280A (anti-PD-L1) Treatment Leads to Clinical Activity in Metastatic Bladder Cancer,” Nature 515(7528): 558-62 (2014) and Massard et al., “Safety and Efficacy of Durvalumab (MEDI4736), an Anti-Programmed Cell Death Ligand-1 Immune Checkpoint Inhibitor, in Patients With Advanced Urothelial Bladder Cancer,” J. Clin. Oncol. 34(26):3119-3125 (2016), which are hereby incorporated by reference in their entirety).

CTLA-4 is a homologous molecule of CD28 that is a competitive antagonist for B7. In the immune recognition process, two signals are required for T lymphocyte expansion and differentiation: the T-cell receptor (TCR) binding to the HLA molecule-peptide complex and an antigen-independent costimulatory signal provided by the B7 (CD80 and Cd86)/CD28 interaction. CTLA-4 has a greater affinity and avidity for B7 than does CD28, and its translocation to the cell surface after T-cell activation results in B7 sequestration and transduction of a negative signal, responsible for T-cell inactivation (Perez-Garcia et al., “CTLA-4 Polymorphisms and Clinical Outcome after Allogeneic Stem Cell Transplantation from HLA-Identical Sibling Donors,” Blood 110(1):461-7 (2007), which is hereby incorporated by reference in its entirety). Ipilimumab (Yervoy) and tremelimumab are immune checkpoint inhibitors that block CTLA-4.

Contacting a tumor having a dysregulated Wnt signaling pathway or administering to a subject an immune checkpoint inhibitor may be carried out simultaneously with contacting or administering a compound of Formula (I). Thus, in some embodiments, said contacting or administering the immune checkpoint inhibitor is carried out before, after, or simultaneously with the administration of the compound of Formula (I). In some embodiments, the immune checkpoint inhibitor and the compound of Formula (I) may be administered by the same route of administration, or the immune checkpoint inhibitor and the compound of Formula (I) may be administered by different routes of administration.

Alternatively, contacting a tumor having a dysregulated Wnt signaling pathway or administering an immune checkpoint inhibitor may be carried out sequentially with contacting or administering a compound of Formula (I). In certain of these embodiments, the immune checkpoint inhibitor and the compound of Formula (I) disclosed herein may be administered as part of a single formulation.

Also contemplated herein is any variation of the above with respect to the sequence of administering the immune checkpoint inhibitor and the compound of Formula (I) in combination. In some embodiments, the immune checkpoint inhibitor is not administered prior to the compound of Formula (I). In other embodiments, the immune checkpoint inhibitor is administered prior to the compound of Formula (I).

In carrying out the methods of the present disclosure, it may be possible for compounds of Formula (I) to be administered as the raw chemical. However, compounds of Formula (I) may also be administered as a pharmaceutical composition. In some embodiments, a pharmaceutical composition is a composition comprising a compound of Formula (I) described herein and a carrier. Thus, in accordance with some embodiments of the present application, there is provided a pharmaceutical composition comprising a compound of Formula (I) or a pharmaceutically acceptable salt or solvate thereof, together with one or more carriers (e.g., a pharmaceutically acceptable carrier(s)) and optionally one or more other therapeutic ingredients. The carrier(s) must be “acceptable” in the sense of being compatible with the other ingredients of the formulation and not deleterious to the recipient thereof.

The immune checkpoint inhibitors, compounds of Formula I, and combinations thereof for use in the methods described herein can be formulated as pharmaceutical compositions suitable for oral, parenteral (including subcutaneous, intradermal, intramuscular, intravenous, and intraarticular), rectal, and topical (including dermal, buccal, sublingual, and intraocular) administration. The most suitable route may depend upon the condition and disorder of the recipient. Formulations may conveniently be presented in unit dosage form and may be prepared by any method well known in the art of pharmacy. Such methods include the step of bringing into association a compound of Formula (I) or pharmaceutically acceptable salts or solvates thereof (an “active ingredient”) with a carrier, which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations suitable for oral administration may be presented as discrete units such as capsules, cachets, or tablets each containing a predetermined amount of the active ingredient; as a powder or granules; as a solution or a suspension in an aqueous liquid or a non-aqueous liquid; or as an oil-in-water liquid emulsion; or a water-in-oil liquid emulsion. The active ingredient may also be presented as a bolus, electuary, or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active, or dispersing agent. Molded tablets may be made by molding in a suitable machine a mixture of the powdered compound moistened with an inert liquid diluent. The tablets may optionally be coated or scored and may be formulated so as to provide sustained, delayed, or controlled release of the active ingredient therein.

The pharmaceutical compositions may include a “pharmaceutically acceptable inert carrier,” and this expression is intended to include one or more inert excipients, which include, for example and without limitation, starches, polyols, granulating agents, microcrystalline cellulose, diluents, lubricants, binders, disintegrating agents, and the like. If desired, tablet dosages of the disclosed compositions may be coated by standard aqueous or nonaqueous techniques. “Pharmaceutically acceptable carrier” also encompasses controlled release means.

Pharmaceutical compositions may also optionally include other therapeutic ingredients, anti-caking agents, preservatives, sweetening agents, colorants, flavors, desiccants, plasticizers, dyes, and the like. Any such optional ingredient must be compatible with the compounds of Formula (I) to insure the stability of the formulation. The composition may contain other additives as needed including, for example, lactose, glucose, fructose, galactose, trehalose, sucrose, maltose, raffinose, maltitol, melezitose, stachyose, lactitol, palatinite, starch, xylitol, mannitol, myoinositol, and the like, and hydrates thereof, and amino acids, for example, alanine, glycine, and betaine, and peptides and proteins, for example, albumen.

Examples of excipients for use as the pharmaceutically acceptable carriers and the pharmaceutically acceptable inert carriers and the aforementioned additional ingredients include, but are not limited to, binders, fillers, disintegrants, lubricants, anti-microbial agents, and coating agents.

Dose ranges for adult humans vary, but may generally be from about 0.005 mg to 10 g/day orally. Tablets or other forms of presentation provided in discrete units may conveniently contain an amount of a compound of Formula (I) and/or an immune checkpoint inhibitor according to the present disclosure that is effective at such dosage or as a multiple of the same, for instance, units containing 5 mg to 500 mg, or around 10 mg to 200 mg. The precise amount of compound administered to a patient will be the responsibility of the attendant physician. However, the dose employed will depend on a number of factors, including the age and sex of the patient, the precise disorder being treated, and its severity.

A dosage unit (e.g., an oral dosage unit) can include from, for example, 1 to 30 mg, 1 to 40 mg, 1 to 100 mg, 1 to 300 mg, 1 to 500 mg, 2 to 500 mg, 3 to 100 mg, 5 to 20 mg, 5 to 100 mg (e.g., 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 150 mg, 200 mg, 250 mg, 300 mg, 350 mg, 400 mg, 450 mg, 500 mg) of a compound of Formula (I).

Additional information about pharmaceutical compositions and their formulation is described in Remington: The Science and Practice of Pharmacy, 20^{th} Edition, 2000, which is hereby incorporated by reference in its entirety.

Compounds of Formula (I) and/or immune checkpoint inhibitors according to the present disclosure can be administered, e.g., by intravenous injection, intramuscular injection, subcutaneous injection, intraperitoneal injection, topical, sublingual, intraarticular (in the joints), intradermal, buccal, ophthalmic (including intraocular), intranasaly (including using a cannula), or by other routes. The compounds can be administered orally, e.g., as a tablet or cachet containing a predetermined amount of the active ingredient, gel, pellet, paste, syrup, bolus, electuary, slurry, capsule, powder, granules, as a solution or a suspension in an aqueous liquid or a non-aqueous liquid, as an oil-in-water liquid emulsion or a water-in-oil liquid emulsion, via a micellar formulation (see, e.g., PCT Publication No. WO 97/11682, which is hereby incorporated by reference in its entirety) via a liposomal formulation (see, e.g., EP Patent No. 736299, PCT Publication No. WO 99/59550, and PCT Publication No. WO 97/13500, which are hereby incorporated by reference in their entirety), via formulations described in PCT Publication No. WO 03/094886 (which is hereby incorporated by reference in its entirety) or in some other form. A compound of Formula (I) and/or immune checkpoint inhibitors according to the present disclosure can also be administered transdermally (i.e., via reservoir-type or matrix-type patches, microneedles, thermal poration, hypodermic needles, iontophoresis, electroporation, ultrasound, or other forms of sonophoresis, jet injection, or a combination of any of the preceding methods (Prausnitz et al., Nature Reviews Drug Discovery 3:115 (2004), which is hereby incorporated by reference in its entirety). A compound of Formula (I) can be administered locally.

Compounds of Formula (I) and/or immune checkpoint inhibitors according to the present disclosure can be administered in the form a suppository or by other vaginal or rectal means. The compounds can be administered in a transmembrane formulation as described in PCT Publication No. WO 90/07923, which is hereby incorporated by reference in its entirety. The compounds can be administered non-invasively via the dehydrated particles, such as those described in U.S. Pat. No. 6,485,706, which is hereby incorporated by reference in its entirety. The compounds can be administered in an enteric-coated drug formulation such as those described in PCT Publication No. WO 02/49621, which is hereby incorporated by reference in its entirety. The compounds can be administered intranasaly using formulations such as those described in U.S. Pat. No. 5,179,079, which is hereby incorporated by reference in its entirety. Formulations suitable for parenteral injection are described in PCT Publication No. WO 00/62759, which is hereby incorporated by reference in its entirety. The compounds of Formula (I) can be administered using the casein formulation described in U.S. Patent Application Publication No. 2003/0206939 and PCT Publication No. WO 00/06108, which are hereby incorporated by reference in their entirety. The compounds can be administered using the particulate formulations described in U.S. Patent Application Publication No. 20020034536, which is hereby incorporated by reference in its entirety.

The compounds, alone or in combination with other suitable components, can be administered by pulmonary route utilizing several techniques including, but not limited to, intratracheal instillation (delivery of solution into the lungs by syringe), intratracheal delivery of liposomes, insufflation (administration of powder formulation by syringe or any other similar device into the lungs), and aerosol inhalation. Aerosols (e.g., jet or ultrasonic nebulizers, metered-dose inhalers (“MDIs”), and dry-Powder inhalers (“DPIs”)) can also be used in intranasal applications. Aerosol formulations are stable dispersions or suspensions of solid material and liquid droplets in a gaseous medium and can be placed into pressurized acceptable propellants, such as hydrofluoroalkanes (HFAs, i.e., HFA-134a and HFA-227, or a mixture thereof), dichlorodifluoromethane (or other chlorofluorocarbon propellants such as a mixture of Propellants 11, 12, and/or 114), propane, nitrogen, and the like. Pulmonary formulations may include permeation enhancers such as fatty acids, and saccharides, chelating agents, enzyme inhibitors (e.g., protease inhibitors), adjuvants (e.g., glycocholate, surfactin, span 85, and nafamostat), preservatives (e.g., benzalkonium chloride or chlorobutanol), and ethanol (normally up to 5% but possibly up to 20%, by weight). Ethanol is commonly included in aerosol compositions as it can improve the function of the metering valve and in some cases also improve the stability of the dispersion.

Pulmonary formulations may also include surfactants which include, but are not limited to, bile salts and those described in U.S. Pat. No. 6,524,557 and references therein, which are hereby incorporated by reference in their entirety. The surfactants described in U.S. Pat. No. 6,524,557, e.g., a C₈-C₁₆ fatty acid salt, a bile salt, a phospholipid, or alkyl saccharide may be advantageous in that some of them also reportedly enhance absorption of the compound in the formulation.

Also suitable are dry powder formulations comprising a therapeutically effective amount of active compound blended with an appropriate carrier and adapted for use in connection with a dry-powder inhaler. Absorption enhancers that can be added to dry powder formulations include those described in U.S. Pat. No. 6,632,456, which is hereby incorporated by reference in its entirety. PCT Publication No. WO 02/080884, which is hereby incorporated by reference in its entirety, describes methods for the surface modification of powders. Aerosol formulations may include those described in U.S. Pat. Nos. 5,230,884 and 5,292,499; PCT Publication Nos. WO 017/8694 and 01/78696; U.S. Patent Application Publication No. 2003/019437, 2003/0165436; and PCT Publication No. WO 96/40089 (which includes vegetable oil), which are hereby incorporated by reference in their entirety. Sustained release formulations suitable for inhalation are described in U.S. Patent Application Publication Nos. 2001/0036481, 2003/0232019, and 2004/0018243 as well as in PCT Publication Nos. WO 01/13891, 02/067902, 03/072080, and 03/079885, which are hereby incorporated by reference in their entirety.

Pulmonary formulations containing microparticles are described in PCT Publication No. WO 03/015750, U.S. Patent Application Publication No. 2003/0008013, and PCT Publication No. WO 00/00176, which are hereby incorporated by reference in their entirety. Pulmonary formulations containing stable glassy state powder are described in U.S. Patent Application Publication No. 2002/0141945 and U.S. Pat. No. 6,309,671, which are hereby incorporated by reference in their entirety. Other aerosol formulations are described in EP Patent No. 1338272, PCT Publication No. WO 90/09781, U.S. Pat. Nos. 5,348,730 and 6,436,367, PCT Publication No. WO 91/04011, and U.S. Pat. Nos. 6,294,153 and 6,290,987, which are hereby incorporated by reference in their entirety, which describe a liposomal based formulation that can be administered via aerosol or other means.

Powder formulations for inhalation are described in U.S. Patent Application Publication No. 2003/0053960 and PCT Publication No. WO 01/60341, which are hereby incorporated by reference in their entirety. The compounds can be administered intranasally as described in U.S. Patent Application Publication No. 2001/0038824, which is hereby incorporated by reference in its entirety.

Solutions of medicament in buffered saline and similar vehicles are commonly employed to generate an aerosol in a nebulizer. Simple nebulizers operate on Bernoulli's principle and employ a stream of air or oxygen to generate the spray particles. More complex nebulizers employ ultrasound to create the spray particles. Both types are well known in the art and are described in standard textbooks of pharmacy.

Other devices for generating aerosols employ compressed gases, usually hydrofluorocarbons and chlorofluorocarbons, which are mixed with the medicament and any necessary excipients in a pressurized container. These devices are likewise described in standard textbooks.

The compounds of Formula (I) and/or the immune checkpoint inhibitors according to the present disclosure can be incorporated into a liposome to improve half-life. The compounds can also be conjugated to polyethylene glycol (“PEG”) chains. Methods for pegylation and additional formulations containing PEG-conjugates (i.e., PEG-based hydrogels, PEG modified liposomes) can be found in Harris and Chess, Nature Reviews Drug Discovery 2:214-221, which is hereby incorporated by reference in its entirety, and the references therein. The compounds can be administered via a nanocochleate or cochleate delivery vehicle (BioDelivery Sciences International). The compounds can be delivered transmucosally (i.e., across a mucosal surface such as the vagina, eye, or nose) using formulations such as that described in U.S. Pat. No. 5,204,108, which is hereby incorporated by reference in its entirety. The compounds can be formulated in microcapsules as described in PCT Publication No. WO 88/01165, which is hereby incorporated by reference in its entirety. The compounds can be administered intra-orally using the formulations described in U.S. Patent Application Publication No. 2002/0055496, PCT Publication No. WO 00/47203, and U.S. Pat. No. 6,495,120, which are hereby incorporated by reference in their entirety. The compounds can be delivered using nanoemulsion formulations described in PCT Publication No. WO 01/91728, which is hereby incorporated by reference in its entirety.

The compounds may be delivered directly to a targeted cell/tissue/organ. Additionally, and/or alternatively, the compounds may be administered to a non-targeted area along with one or more agents that facilitate migration of the compounds to (and/or uptake by) a targeted tissue, organ, or cell. As will be apparent to one of ordinary skill in the art, the compound itself can be modified to facilitate its transport to a target tissue, organ, or cell, including its transport across the blood-brain barrier; and/or to facilitate its uptake by a target cell (e.g., its transport across cell membranes).

In some embodiments, contacting a tumor having a dysregulated Wnt signaling pathway with a compound of Formula (I) and/or an immune checkpoint inhibitor according to the present disclosure is carried out in vitro.

In some embodiments, contacting a tumor having a dysregulated Wnt signaling pathway with a compound of Formula (I) and/or an immune checkpoint inhibitor according to the present disclosure is carried out in vivo in a subject having the tumor with a dysregulated Wnt signaling pathway. When contacting with a compound of Formula (I) and/or an immune checkpoint inhibitor according to the present disclosure is carried out in vivo in a subject having the tumor comprising a dysregulated Wnt signaling pathway, contacting may be carried out by administering the compound of Formula (I) and/or an immune checkpoint inhibitor according to the present disclosure to the subject.

Contacting a subject with a compound or administering of compounds and/or pharmaceutical compositions to a subject may involve administering therapeutically effective amounts, which means an amount of compound effective in treating the stated conditions and/or disorders in a subject. Such amounts generally vary according to a number of factors well within the purview of ordinarily skilled artisans. These include, without limitation, the particular subject, as well as the subject's age, weight, height, general physical condition, and medical history, the particular compound used, as well as the carrier in which it is formulated and the route of administration selected for it; and, the nature and severity of the condition being treated.

Administering typically involves administering pharmaceutically acceptable dosage forms, which means dosage forms of compounds described herein, and includes, for example, tablets, dragees, powders, elixirs, syrups, liquid preparations, including suspensions, sprays, inhalants tablets, lozenges, emulsions, solutions, granules, capsules, and suppositories, as well as liquid preparations for injections, including liposome preparations. Techniques and formulations generally may be found in Remington's Pharmaceutical Sciences, Mack Publishing Co., Easton, Pa., latest edition, which is hereby incorporated by reference in its entirety.

Administering may be carried out orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes. Compounds may be administered alone or with suitable pharmaceutical carriers, and can be in solid or liquid form, such as tablets, capsules, powders, solutions, suspensions, or emulsions.

As used herein, the term “subject” refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cat, or a dog. In some embodiments, the subject is a research animal. In some embodiments, the subject is genetically engineered, e.g., a genetically engineered non-human subject.

In some embodiments, when the subject has a tumor having a dysregulated Wnt signaling pathway comprising a mutation in one or more genes selected from the group consisting of CTNNB1, APC, AXIN1, AXIN2, GSK3B, LGR5, RNF43, ZNRF3, LRP6, FBXW7, and TCF7L2, the subject may be identified as having a tumor cell comprising cells containing a CTNNB1, APC, AXIN1, AXIN2, GSK3B, LGR5, RNF43, ZNRF3, LRP6, FBXW7, and TCF7L2mutation by testing tumor cells for such mutations. This may occur, for example and without limitation, through sequencing (by targeted sequencing, droplet digital PCR, etc.).

In some embodiments, a subject with a tumor comprising a dysregulated Wnt signaling pathway is identified prior to administering a compound of Formula (I).

In some embodiments, identifying a subject with a tumor comprising a dysregulated Wnt signaling pathway involves obtaining a tissue sample from the tumor and testing the sample for a marker associated with dysregulated Wnt signaling pathway.

“Obtaining a tissue sample” as used herein, refers to obtaining possession of a sample by directly acquiring or indirectly acquiring the sample. Directing acquiring a sample means performing a process (e.g., performing a physical method such as surgery, biopsy, or extraction) to obtain the sample. Indirectly acquiring a sample refers to receiving the sample from another party or source (e.g., a third party laboratory that directly acquired the sample). Methods described herein can include obtaining a tissue sample from a tumor.

The source of the tissue sample can be solid tissue, as from a fresh, frozen, and/or preserved organ tissue sample, biopsy, or aspirate; blood or any blood constituents; bodily fluids such as cerebral spinal fluid, amniotic fluid, peritoneal fluid, or interstitial fluid; or cells from any time in gestation or development of the subject. In some embodiments, the tissue sample is from a tumor. The tissue sample can contain compounds that are not naturally intermixed with the tissue in nature, such as preservatives, anticoagulants, buffers, fixatives, nutrients, antibiotics, or the like. The sample may be preserved as a frozen sample or as fomaldehyde- or paraformaldehyde-fixed paraffin-embedded (“FFPE”) tissue preparation. For example, the sample can be embedded in a matrix, e.g., an FFPE block or frozen sample. Typically, the sample is a tumor sample, e.g., includes one or more premalignant or malignant cells. In certain embodiments, the sample, e.g., the tumor sample, is acquired from a solid tumor, a soft tissue tumor, or a metastatic lesion. In some embodiments, the sample, e.g., the tumor sample, includes tissue or cells from a surgical margin. In some embodiments, the sample, e.g., the tumor sample, includes one or more circulating tumor cells (e.g., acquired from a blood sample).

Identifying a tumor having a dysregulated Wnt signaling pathway can be carried out using methods that are well known in the art. In some embodiments, detecting or identifying a dysregulated Wnt signaling pathway comprises identifying or detecting a mutation in a one or more genes selected from the group consisting of CTNNB1, APC, AXIN1, AXIN2, GSK3B, LGR5, RNF43, ZNRF3, LRP6, FBXW7, and TCF7L2. Detecting or identifying a mutation in a gene can be carried out, in some embodiments, by sequencing at least a portion of the nucleotide sequence of the gene comprising the mutation. This can be done by direct sequencing, including direct sequencing of one or more gene regions comprising possible mutations, from a tissue sample obtained from the tumor of a subject. Direct sequencing assays typically involve isolating a DNA sample from the subject using any suitable method known in the art, and cloning the region of interest to be sequenced into a suitable vector for amplification by growth in a host cell (e.g., bacteria) or direct amplification by PCR or other amplification assay. Following amplification, the DNA can be sequenced using any suitable method. One sequencing method involves high-throughput next generation sequencing (“NGS”) to identify genetic variation. Various NGS sequencing chemistries are available and suitable for use in carrying out the methods disclosed herein, including pyrosequencing (Roche® 454), sequencing by reversible dye terminators (Illumina® HiSeq, Genome Analyzer and MiSeq systems or the like), sequencing by sequential ligation of oligonucleotide probes (Life Technologies® SOLiD), and hydrogen ion semiconductor sequencing (e.g., Life Technologies®, Ion Torrent™, Oxford Nanopore, or PacBio IsoSeq platforms). Alternatively, classic sequencing methods, such as the Sanger chain termination method or Maxam-Gilbert sequencing, which are well known to those of ordinary skill in the art, can be used to carry out the methods disclosed herein.

In some embodiments, the dysregulated Wnt signaling pathway is identified or detected in a hybridization assay utilizing one or more oligonucleotide probes comprising a nucleotide sequence that is complementary to a nucleic acid molecule encoding for CTNNB1, APC, AXIN1, AXIN2, GSK3B, LGR5, RNF43, ZNRF3, LRP6, FBXW7, and TCF7L2. In a hybridization assay, the presence or absence of a gene mutation is determined based on the hybridization of one or more oligonucleotide probes to one or more nucleic acid molecules in a sample from the subject. The oligonucleotide probe or probes comprise a nucleotide sequence that is complementary to at least the region of the gene that contains the identified mutation. The oligonucleotide probes are designed to be complementary to the wild type, non-mutant nucleotide sequence and/or the mutant nucleotide sequence of the one or more genes to effectuate the detecting of the presence or the absence of the mutation in the sample from the subject upon contacting the sample with the oligonucleotide probe(s).

A variety of hybridization assays that are known in the art are suitable for use in the methods disclosed herein. These methods include, without limitation, direct hybridization assays, such as northern blot or Southern blot (see, e.g., Ausabel et al., Current Protocols in Molecular Biology, John Wiley & Sons, NY (1991), which is hereby incorporated by reference in its entirety). Alternatively, direct hybridization can be carried out using an array based method where oligonucleotide probe(s) designed to be complementary to a particular non-mutant or mutant gene region are affixed to a solid support. A labeled DNA or cDNA sample from the subject is contacted with the array containing the oligonucleotide probe(s), and hybridization of nucleic acid molecules from the sample to their complementary oligonucleotide probes on the array surface is detected. Examples of direct hybridization array platforms include, without limitation, the Affymetrix GeneChip or SNP arrays and Illumina's Bead Array.

In another embodiment, identifying is carried out with an amplification-based assay which amplifies a nucleic acid molecule comprising a Wnt signaling pathway component. Amplification based assays include assays such as molecular beacon assays, nucleic acid arrays, and allele-specific PCR. Other common genotyping methods include, but are not limited to, restriction fragment length polymorphism assays; primer extension assays, such as allele-specific primer extension (e.g., Illumina© Infinium© assay), arrayed primer extension (see Krjutskov et al., “Development of a Single Tube 640-plex Genotyping Method for Detection of Nucleic Acid Variations on Microarrays,” Nucleic Acids Res. 36(12):e75 (2008), which is hereby incorporated by reference in its entirety), homogeneous primer extension assays, primer extension with detection by mass spectrometry (e.g., Sequenom© iPLEX SNP genotyping assay) (see Zheng et al., “Cumulative Association of Five Genetic Variants with Prostate Cancer,” N. Eng. J. Med. 358(9):910-919 (2008), which is hereby incorporated by reference in its entirety), multiplex primer extension sorted on genetic arrays; flap endonuclease assays (e.g., the Invader© assay) (see Olivier “The Invader Assay for SNP Genotyping,” Mutat. Res. 573(1-2):103-10 (2005), which is hereby incorporated by reference in its entirety); 5′ nuclease assays, such as the TaqMan® assay (see U.S. Pat. No. 5,210,015 to Gelfand et al. and U.S. Pat. No. 5,538,848 to Livak et al., which are hereby incorporated by reference in their entirety); and oligonucleotide ligation assays, such as ligation with rolling circle amplification, homogeneous ligation, OLA (see U.S. Pat. No. 4,988,617 to Landgren et al., which is hereby incorporated by reference in its entirety), multiplex ligation reactions followed by PCR, wherein zipcodes are incorporated into ligation reaction probes, and amplified PCR products are determined by electrophoretic or universal zipcode array readout (see U.S. Pat. Nos. 7,429,453 and 7,312,039 to Barany et al., which are hereby incorporated by reference in their entirety). Such methods may be used in combination with detection mechanisms such as, for example, luminescence or chemiluminescence detection, fluorescence detection, time-resolved fluorescence detection, fluorescence resonance energy transfer, fluorescence polarization, mass spectrometry, and electrical detection.

According to one embodiment, once a dysregulated Wnt signaling pathway mutation is identified, a compound of Formula (I) may be administered to the subject.

Cancers amenable to the treatment method of the present invention include, without limitation, hepatocellular carcinoma; endometrial cancer; colorectal cancer; liver adenoma; LIAD; hepatocellular adenoma; melanoma; or cancer of the uterus, pancreas, prostate, stomach, bladder, anus, or esophagus.

In some embodiments, the cancer treated in the methods of the present application is hepatocellular carcinoma.

In some embodiments, the cancer treated is colorectal cancer.

As described herein supra, the subject may be a mammalian subject, e.g., a human subject.

In some embodiments, the subject is treated for colorectal cancer; gastric cancer; endometrial cancer; lung cancer; liver cancer; hepatocellular carcinoma; hepatocellular adenoma; hepatoblastoma; melanoma; bladder carcinoma; pilomatrixoma; ovarian cancer; medulloblastoma; adenocortical carcinoma; pancreatic cancer; NSCLC; liver adenoma; LIAD; hepatoblastoma; or cancer of the uterus, pancreas, prostate, stomach, bladder, anus, or esophagus.

As used herein, the term “reduce” or “reduces” refers to its meaning as is generally accepted in the art. With reference to compounds according to the present disclosure, “reduce” or “reduces” generally refers to a suppression in the transcription and/or translation of a Wnt target gene or in the levels of the Wnt target gene product relative to the transcription and/or translation of the Wnt target gene observed in the absence of the compound of Formula (I) according to the present disclosure. In some embodiments, the reduction in the transcription and/or translation of a Wnt target gene or in the levels of the Wnt target gene product is at least 10%, at least 2000, at least 300%, at least 40%, at least 50%, at least 60%, at least 70%, at least 80%, at least 90, at least 950, at least 96, at least 9700 at least 98, at least 9900 up to 100% (i.e., no detectable transcription and/or translation) or a reduction of at least 2-fold, at least 5-fold, at least 10-fold, at least 20-fold, at least 30-fold, at least 40-fold, at least 50-fold, or more relative to that observed in the absence of the compounds according to the present disclosure. Exemplary Wnt regulated proteins are provided in Table 4 below.

TABLE 4
Exemplary Wnt Regulated Proteins

| | Organism/ | up/ | |
|---|---|---|---|
| Gene | system | down | Ref.* |
| c-myc | human colon | up | He et al., “Identification of c-MYC as a Target of the APC Pathway,” |
| | cancer | | Science 281: 1509-12 (1998) |
| n-myc | mesenchyme | up | Ten Berge et al., “Wnt and FGF Signals Interact to Coordinate |
| | limbs | | Growth with Cell Fate Specification During Limb Development,” |
| | | | Development 135(19): 3247-3257 (2008) |
| Cyclin D | human colon | up | Tetsu & McCormick, “Beta-Catenin Regulates Expression of Cyclin |
| | cancer | | D1 in Colon Carcinoma Cells,” Nature 398(6726): 422-426 (1999) |
| | | | and Shtutman et al., “The Cyclin D1 Gene is a Target of the Beta- |
| | | | Catenin/LEF-1 Pathway,” PNAS 96(10): 5522-5527 (1999) |
| Tcf-1 | human colon | up | Roose et al., “Synergy between Tumor Suppressor APC and the |
| | cancer | | Beta-Catenin-Tcf4 Target Tcf1,” Science 285(5435): 1923-1926 |
| | | | (1999) |
| LEF1 | human colon | up | Hovanes et al., “Beta-Catenin-Sensitive Isoforms of Lymphoid |
| | cancer | | Enhancer Factor-1 are Selectively Expressed in Colon Cancer,” Nat. |
| | | | Genet. 28(1): 53-57 (2001) and Filali et al., “Wnt-3 A/Beta-Catenin |
| | | | Signaling Induces Transcription from the LEF-1 Promoter,” J. Biol. |
| | | | Chem. 277(36): 33398-33410 (2002) |
| PPARdelta | human colon | up | He et al., “PPARdelta is an APC-Regulated Target of Nonsteroidal |
| | cancer | | Anti-Inflammatory Drugs,” Cell 99(3): 335-345 (1999) |
| c-jun | human colon | up | Mann et al., “Target Genes of Beta-Catenin-T Cell- |
| | cancer | | Factor/Lymphoid-Enhancer-Factor Signaling in Human Colorectal |
| | | | Carcinomas,” PNAS 96(4): 1603-1608 (1999) |
| fra-1 | human colon | up | Mann et al., “Target Genes of Beta-Catenin-T Cell- |
| | cancer | | Factor/Lymphoid-Enhancer-Factor Signaling in Human Colorectal |
| | | | Carcinomas,” PNAS 96(4): 1603-1608 (1999) |
| uPAR | human colon | up | Mann et al., “Target Genes of Beta-Catenin-T Cell- |
| | cancer | | Factor/Lymphoid-Enhancer-Factor Signaling in Human Colorectal |
| | | | Carcinomas,” PNAS 96(4): 1603-1608 (1999) |
| matrix | human colon | up | Brabletz et al., “Beta-Catenin Regulates the Expression of the Matrix |
| metallo- | cancer | | Metalloproteinase-7 in Human Colorectal Cancer,” Am. J. Pathol. |
| proteinase | | | 155(4): 1033-1038 (1999) and Crawford et al., “The |
| MMP-7 | | | Metalloproteinase Matrilysin is a Target of Beta-Catenin |
| | | | Transactivation in Intestinal Tumors,” Oncogene 18(18): 2883-2891 |
| | | | (1999) |
| Axin-2 | human colon | up | Yan et al., “Elevated Expression of Axin2 and hnkd mRNA Provides |
| | cancer | | Evidence that Wnt/Beta -Catenin Signaling is Activated in Human |
| | | | Colon Tumors,” PNAS 98(26): 14973-14978 (2001); |
| | | | Lustig et al., “Negative Feedback Loop of Wnt Signaling through |
| | | | Upregulation of Conductin/Axin2 in Colorectal and Liver Tumors,” |
| | | | Mol. Cell Biol. 22(4): 1184-1193 (2002); and Jho et al., “Wnt/Beta- |
| | | | Catenin/Tcf Signaling Induces the Transcription of Axin2, a |
| | | | Negative Regulator of the Signaling Pathway,” Mol. Cell. Biol. |
| | | | 22(4): 1172-1183 (2002) |
| Nr-CAM | human colon | up | Conacci-Sorrell et al., “NR_-CAM is a Target Gene of the Beta- |
| | cancer | | Catenin/LEF-1 Pathway in Melanoma and Colon Cancer and its |
| | | | Expression Enhances Motility and Confers Tumorigenesis,” Genes |
| | | | Dev. 16(16): 2058-2072 (2002) |
| ITF-2 | human colon | up | Kolligs et al., “ITF-2, a Downstream Target of the Wnt/TCF |
| | cancer | | Pathway, is Activated in Human Cancers with Beta-Catenin Defects |
| | | | and Promotes Neoplastic Transformation,” Cancer Cell 1(2): 145-155 |
| | | | (2002) |
| Gastrin | human colon | up | Koh et al., “Gastrin is a Target of the Beta-Catenin/TCF-4 Growth- |
| | cancer | | Signaling Pathway in a Model of Intestinal Polyposis,” J. Clin. |
| | | | Invest. 106(4): 533-539 (2000) |
| CD44 | human colon | up | Wielenga et al., “Expression of CD44 in Apc and Tcf Mutant Mice |
| | cancer | | Implies Regulation by the WNT Pathway,” Am. J. Pathol. |
| | | | 154(2): 515-523 (1999) |
| EphB/ | human colon | up/ | Batlle et al., “Beta-Catenin and TCF Mediate Cell Positioning in the |
| ephrin-B | cancer | down | Intestinal Epithelium by Controlling the Expression of |
| | | | EphB/ephrinB,” Cell 111(2): 251-263 (2002) |
| BMP4 | human colon | up | Kim et al., “Oncogenic Beta-Catenin is Required for Bone |
| | cancer | | Morphogenetic Protein 4 Expression in Human Cancer Cells,” |
| | | | Cancer Res. 65(10): 2744-2748 (2002) |
| claudin-1 | human colon | up | Miwa et al., “Involvement of Claudin-1 in the Beta-Catenin/Tcf |
| | cancer | | Signaling Pathway and its Frequent Upregulation in Human |
| | | | Colorectal Cancers,” Oncol. Res. 12(11-12): 469-476 (2001) |
| Survivin | human colon | up | Zhang et al., “Evidence that APC Regulates survivin Expression: A |
| | cancer | | Possible Mechanism Contributing to the Stem Cell Origin of Colon |
| | | | Cancer,” Cancer Res. 61(24): 8664-8667 (2001) |
| VEGF | human colon | up | Zhang et al., “Regulation of Vascular Endothelial Growth Factor by |
| | cancer | | the Wnt and K-ras Pathways in Colonic Neoplasia,” Cancer Res. |
| | | | 61(16): 6050-6054 (2001) |
| FGF18 | human colon | up | Shimokawa et al., “Involvement of the FGF18 Gene in Colorectal |
| | cancer | | Carcinogenesis, as a Novel Downstream Target of the Beta- |
| | | | Catenin/T-Cell Factor Complex,” Cancer Res. 63(19): 6116-6120 |
| | | | (2003) |
| Hath 1 | human colon | down | Leow et al., “Hathl, Down-Regulated in Colon Adenocarcinomas, |
| | cancer | | Inhibits Proliferation and Tumorigenesis of Colon Cancer Cells,” |
| | | | Cancer Res. 64(17): 6050-6057 (2004) |
| Met | human colon | up | Boon et al., “Wnt Signaling Regulates Expression of the Receptor |
| | cancer | | Tyrosine Kinase met in Colorectal Cancer,” Cancer Res. |
| | | | 62(18): 5126-5128 (2002) |
| endothelin-1 | human colon | up | Kim et al., “Beta-Catenin Activates the Growth Factor Endothelin-1 |
| | cancer | | in Colon Cancer Cells,” Oncogene 24(4): 597-604 (2005) |
| c-myc | human colon | up | Jung et al., “Identification of MYCBP as a Beta-Catenin/LEF-1 |
| binding | cancer | | Target using DNA Microarray Analysis,” Life Sci. 77(110: 1249-1262 |
| protein | | | (2005) |
| L1 neural | human colon | up | Gavert et al., “L1, a Novel Target of Beta-Catenin Signaling, |
| adhesion | cancer | | Transforms Cells and is Expressed at the Invasive Front of Colon |
| | | | Cancers,” J. Cell. Biol. 168(4): 633-642 (2005) |
| Id2 | human colon | up | Rockman et al., “Id2 is a Target of the Beta-Catenin/T Cell Factor |
| | cancer | | Pathway in Colon Carcinoma,” J. Biol. Chem. 276(48): 45113-45119 |
| | | | (2001) and Willert et al., “A Transcriptional Response to Wnt Protein |
| | | | in Human Embryonic Carcinoma Cells,” BMC Develop. Biol. 2: 8 |
| | | | (2002) |
| Jagged | human colon | up | Rodilla et al., “Jagged1 is the Pathological Link Between Wnt and |
| | cancer | | Notch Pathways in Colorectal Cancer,” PNAS 106(15): 6315-6320 |
| | | | (2009) |
| Msl1 | human colon | up | Spears et al., “Novel Double-Negative Feedback Loop between |
| | cancer | | Adenomatous Polyposis coli and Musashil in Colon Epithelia,” J. |
| | | | Biol. Chem. 286(7): 4946-4950 (2011) |
| Tiam1 | Colon tumors | | Malliri et al., “The rac Activator Tiaml is a Wnt-Responsive Gene |
| | | | that Modifies Intestinal Tumor Development,” J. Biol. Chem. |
| | | | 281(1): 543-548 (2006) |
| Nitric Oxide | Hepg2 cells | up | Du et al., “Regulation of Human Nitric Oxide Synthase 2 Expression |
| Synthase 2 | | | by Wnt Beta-Catenin Signaling,” Cancer Res. 66(14): 7024-7031 |
| | | | (2006) |
| Telomerase | ES, other | up | Hoffmyer et al., “Wnt/β-catenin Signaling Regulates Telomerase in |
| | stem | | Stem Cells and Cancer Cells,” Science 336(6088): 1594-1554 (2012) |
| Dickkopf | Various cells, | up | Niida et al., “DKK1, A Negative Regulator of Wnt Signaling, is a |
| | tumors | | Target of the Beta-Catenin/TCF Pathway,” Oncogene 23(52): 8520- |
| | | | 8526 (2004); Gonzalez-Sancho et al., “The Wnt Antagonist |
| | | | DICKKOPF-1 Gene is a Downstream Target of Beta-Catenin/TCF |
| | | | and is DowNR_egulated in Human Colon Cancer,” Oncogene |
| | | | 24(6): 1098-1103 (2005); and Chamorro et al., “FGF-20 and DKK1 |
| | | | are Transcriptional Targets of Beta-Catenin and FGF-20 is |
| | | | Implicated in Cancer and Development,” EMBO J. 24(1): 73-84 |
| | | | (2005) |
| FGF9 | ovarian | up | Hendrix et al., “Fibroblast Growth Factor 9 has Oncogenic Activity |
| | endome- | | and is a Downstream Target of Wnt Signaling in Ovarian |
| | trioid adeno- | | Endometrioid Adenocarcinomas,” Cancer Res. 66(3): 1354-1362 |
| | carcinoma | | (2006) |
| LBH | breast cancer | up | Rieger et al., “The Embryonic Transcription Cofactor LBH is a |
| | | | Direct Target of the Wnt Signaling Pathway in Epithelial |
| | | | Development and in Aggressive Basal Subtype Breast Cancers,” |
| | | | Mol. Cell Biol. 30(17): 4267-4279 (2010) |
| FGF20 | Various cells, | | Chamorro et al., “FGF-20 and DKK1 are Transcriptional Targets of |
| | tumors | | Beta-Catenin and FGF-20 is Implicated in Cancer and |
| | | | Development,” EMBO J. 24(1): 73-84 (2005) |
| LGR5/ | Intestine | up | Barker et al., “Identification of Stem Cells in Small Intestine and |
| GPR49 | | | Colon by Marker Gene Lgr5,” Nature 449(7165): 1003-1007 (2007) |
| Sox9 | Intestine | up | Blache et al., “SOX9 is an Intestine Crypt Transcription Factor, is |
| | | | Regulated by the Wnt Pathway, and Represses the CDX2 and MUC2 |
| | | | Genes,” J. Cell Biol. 166(1): 37-47 (2004) |
| Sox9 | Mesen- | down | Hill et al., “Canonical Wnt/Beta-Catenin Signaling Prevents |
| | chyme | | Osteoblasts from Differentiating into Chondrocytes,” Dev. Cell |
| | | | 8(5): 727-738 (2005); Day et al., “Wnt/Beta-Catenin Signaling in |
| | | | Mesenchymal Progenitors Controls Osteoblast and Chondrocyte |
| | | | Differentiation During Vertebrate Skeletogenesis,” Dev. Cell |
| | | | 8(5): 739-750 (2005); and Yano et al., “The Canonical Wnt Signaling |
| | | | Pathway Promotes Chondrocyte Differentiation in a Sox9-Dependent |
| | | | Manner,” Biochem. Biophys. Res. Commun. 333(4): 1300-1308 |
| | | | (2005) |
| Sox17 | gastrointest- | up | Du et al., “Induction and Down-Regulation of Sox17 and its Possible |
| | inal tumors | | Roles During the Course of Gastrointestinal Tumorigenesis,” |
| | | | Gastroenterology 137(4): 1346-1357 (2009) |
| Runx2 | chondro- | up | Dong et al., “Wnt Induction of Chondrocyte Hypertrophy through the |
| | cytes | | Runx2 Transcription Factor,” J. Cell Physiol. 208(1): 77-76 (2006) |
| Gremlin | fibroblasts | up | Klapholz-Brown et al., “Transcriptional Program Induced by Wnt |
| | | | Protein in Human Fibroblasts Suggests Mechanisms for Cell |
| | | | Cooperativity in Defining Tissue Microenvironments,” PLos One |
| | | | 2(9): e945 (2007) |
| SALL4 | | | Bohm et al., “SALL4 is Directly Activated by TCF/LEF in the |
| | | | Canonical Wnt Signaling Pathway,” Biochem. Biophys. Res. |
| | | | Commun. 348(3): 898-907 (2006) |
| RANK | Osteoblasts | down | Spencer et al., “Wnt Signalling in Osteoblasts Regulates Expression |
| ligand | | | of the Receptor Activator of NFkappaB Ligand and Inhibits |
| | | | Osteoclastogenesis In Vitro,” J. Cell Sci. 119(Pt. 7): 1283-1296 |
| | | | (2006) |
| Osteoproteg | Osteoblasts | up | Glass et al., “Canonical Wnt Signaling in Differentiated Osteoblasts |
| erin | | | Controls Osteoclast Differentiation,” Dev. Cell 8(5): 751-764 (2005) |
| CCN1/Cyr61 | Osteoblasts | up | Si et al., “CCN1/Cyr61 is Regulated by the Canonical Wnt Signal |
| | | | and Plays an Important Role in Wnt3A-Induced Osteoblast |
| | | | Differentiation of Mesenchymal Stem Cells,” Mol. Cell Biol. |
| | | | 26(8): 2955-2964 (2006) |
| Pituitary | esophageal | | Zhou et al., “Overexpression of Human Pituitary Tumor |
| tumor | squamous | | Transforming Gene (hPTTG), is Regulated by Beta-Catenin /TCF |
| transforming | cell | | Pathway in Human Esophageal Squamous Cell Carcinoma,” Int. J. |
| gene | carcinoma | | Cancer 113(6): 891-898 (2005) |
| (PTTG) | | | |
| Delta-like 1 | somites | | Galceran et al., “LEF1-Mediated Regulation of Delta-like1 links Wnt |
| | | | and Notch Signaling in Somitogenesis,” Genes Dev. 18(22): 2718- |
| | | | 2723 (2004) and Hofmann et al., “WNT Signaling, in Synergy with |
| | | | T/TBX6, Controls Notch Signaling by Regulating Dll1 Expression in |
| | | | the Presomitic Mesoderm of Mouse Embryos,” Genes Dev. |
| | | | 18(22): 2712-2717 (2004) |
| FoxN1 | thymus | yes | Balciunaite et al., “Wnt Glycoproteins Regulate the Expression of |
| | | | FoxN1, the Gene Defective in Nude Mice,” Nat. Immunol. |
| | | | 3(11): 1102-1108 (2002) |
| matrix | Human | | Marchenko et al., “Promoter Characterization of the Novel Human |
| metalloprote | | | Matrix Metalloproteinase-26 Gene: Regulation by the T-Cell Factor- |
| inase-26 | | | 4 Implies Specific Expression of the Gene in Cancer Cells of |
| | | | Epithelial Origin,” Biochem. J. 353(Pt 2): 253-262 (2002) |
| nanog | ES | | Pereira et al., “Repression of Nanog Gene Transcription by Tcf3 |
| | | | Limits Embryonic Stem Cell Self-Renewal,” Mol. Cell Biol. |
| | | | 26(20): 7479-7491 (2006) and Cole et al., “Tcf3 is an Integral |
| | | | Component of the Core Regulatory Circuitry of Embryonic Stem |
| | | | Cells,” Genes Dev. 22(6): 746-755 (2008) |
| Oct 4 | ES | up | Cole et al., “Tcf3 is an Integral Component of the Core Regulatory |
| | | | Circuitry of Embryonic Stem Cells,” Genes Dev. 22(6): 746-755 |
| | | | (2008) |
| snail | ES/EB | up | Ten Berge et al., “Wnt Signaling Mediates Self-Organization and |
| | | | Axis Formation in Embryoid Bodies,” Cell Stem Cell 3(5): 508-518 |
| | | | (2008) |
| Fibronectin | ES/EB | up | Ten Berge et al., “Wnt Signaling Mediates Self-Organization and |
| | | | Axis Formation in Embryoid Bodies,” Cell Stem Cell 3(5): 508-518 |
| | | | (2008) |
| Frizzled 7 | EC cells | up | Willert et al., “A Transcriptional Response to Wnt Protein in Human |
| | | | Embryonic Carcinoma Cells,” BMC Dev. Biol. 2: 8 (2002) |
| Follistatin | EC cells, | up | Willert et al., “A Transcriptional Response to Wnt Protein in Human |
| | ovary | | Embryonic Carcinoma Cells,” BMC Dev. Biol. 2: 8 (2002) and Yao et |
| | | | al., “Follistatin Operates Downstream of Wnt4 in Mammalian Ovary |
| | | | Organogenesis,” Dev. Dyn. 230(2): 210-215 (2004) |
| Wnt3a | EC cells | | Zhang et al., “Secreted Frizzled Related Protein 2 Protects Cells from |
| | | | Apoptosis by Blocking the Effect of Canonical Wnt3a,” J. Mol. Cell |
| | | | Cardiol. 46(3): 370-377 (2009) |
| Islet1 | Cardiac cells | up | Lin et al., “Beta-Catenin Directly Regulates Islet1 Expression in |
| | | | Cardiovascular Progenitors and is Required for Multiple Aspects of |
| | | | Cardiogenesis,” PNAS 104(22): 9313-9318 (2007) |
| Twist | Wnt1 induced | up | Howe et al., “Twist is Up-Regulated in Response to Wnt1 and |
| | mammary | | Inhibits Mouse Mammary Cell Differentiation,” Cancer Res. |
| | cancer | | 63(8): 1906-1913 (2003) |
| Stromelysin | Wnt-1 | up | Prieve & Moon, “Stromelysin-1 and Mesothelin are Differentially |
| | transformed | | Regulated by Wnt-5a and Wnt-1 in C57mg Mouse Mammary |
| | mouse cells | | Epithelial Cells,” BMC Dev. Biol. 3: 2 (2003) |
| WISP | Wnt-1 | up | Xu et al., “WISP-1 is a Wnt-1- and Beta-Catenin-Responsive |
| | transformed | | Oncogene,” Genes Dev. 14(5): 585-595 (2000) |
| | mouse cells | | |
| Brachyury | Mouse (Wnt- | up | Arnold et al., “Brachyury is a Target Gene of the Wnt/Beta-Catenin |
| (Tbox1) | 3A) | | Signaling Pathway,” Mech. Dev. 91(1-2): 249-258 (2000) |
| Tbx3 | Human | up | Renard et al., “Tbx3 is a Downstream Target of the Wnt/Beta- |
| (Tbox3) | | | Catenin Pathway and a Critical Mediator of Beta-Catenin Survival |
| | | | Functions in Liver Cancer,” Cancer Res. 67(3): 901-910 (2007) |
| Nkx2.2 | Neural tube | down | Lei et al., “Wnt Signaling Inhibitors Regulate the Transcriptional |
| | | | Response to Morphogenetic Shh-Gli Signaling in the Neural Tube,” |
| | | | Dev. Cell 11(3): 325-337 (2006) |
| Gbx2 | Neural Crest | up | Li et al., “The Posteriorizing Gene Gbx2 is a Direct Target of Wnt |
| | | | Signalling and the Earliest Factor in Neural Crest Induction,” |
| | | | Development 136(19): 3267-3278 (2009) |
| Cacnalg | Neuron | up | Wisniewska et al., “LEF1/Beta-Catenin Complex Regulates |
| | | | Transcription of the Cav3.1 Calcium Channel Gene (Cacnalg) in |
| | | | Thalamic Neurons of the Adult Brain,” J. Neruosci. 30(14): 4957- |
| | | | 4969 (2010) |
| WISP-1, | 3T3-L1 | up | Longo et al., “Wnt Signaling Protects 3T3-LI Preadipocytes from |
| WISP-2, | Preadipocytes | | Apoptosis through Induction of Insulin-Like Growth Factors,” J. |
| IGF-II, | | | Biol. Chem. 277(41): 38239-38244 (2002) |
| Proliferin-2, | | | |
| Proliferin-3, | | | |
| Emp, IGF-I, | | | |
| VEGF-C, | | | |
| MDR1, | | | |
| COX-2, IL-6 | | | |
| betaTrCP | | up | Spiegelman et al., “Wnt/Beta-Catenin Signaling Induces the |
| | | | Expression and Activity of BetaTrCP Ubiquitin Ligase Receptor,” |
| | | | Mol. Cell 5(5): 877-882 (2000) |
| Cdc25 | Sarcoma cells | up | Viyajakumar et al., “Wnt Signaling is Activated at High Frequency |
| | | | and Drives Proliferation of Multiple Human Sarcoma Subtypes |
| | | | through a TCF/ß-Catenin Target Gene, CDC25A,” Cancer Cell |
| | | | 19(5): 601-612 (2011) |
| Pitx2 | pituitary | up | Kioussi et al., “Identification of a Wnt/Dvl/Beta-Catenin --> Pitx2 |
| | | | Pathway Mediating Cell-Type-Specific Proliferation During |
| | | | Development,” Cell 111(5): 673-685 (2002) |
| EGF | Liver | up | Tan et al., “Epidermal Growth Factor Receptor: A Novel Target of |
| receptor | | | the Wnt/Beta-Catenin Pathway in Liver,” Gastroenterology |
| | | | 129(1): 285-302 (2005) |
| P16ink4A | Melanocytes | down | Delmas et al., “Beta-Catenin Induces Immortalization of |
| | | | Melanocytes by Suppressing p16INK4a Expression and Cooperates |
| | | | with N-Ras in Melanoma Development,” Genes Dev. 21(22): 2923- |
| | | | 2935 (2007) |
| CTLA-4 | Melanomas | up | Shah et al., “CTLA-4 is a Direct Target of Wnt/Beta-Catenin |
| | | | Signaling and is Expressed in Human Melanoma Tumors,” J. Invest. |
| | | | Dermatol. 128(12): 2870-2879 (2008) |
| Interleukin8 | Endothelial | | Masckauchan et al., “Wnt/Beta-Catenin Signaling Induces |
| | cells | | Proliferation, Survival and Interleukin-8 in Human Endothelial |
| | | | Cells,” Angiogenesis 8(1): 43-51 (2005) |
| versican | vascular | up | Rahmani et al., “Regulation of the Versican Promoter by the Beta- |
| | smooth | | Catenin-T-Cell Factor Complex in Vascular Smooth Muscle Cells,” |
| | muscle cells | | J. Biol. Chem. 280(13): 13019-13028 (2005) |
| Tnfrsf19 | Somitic | up | Buttitta et al., “Microarray Analysis of Somitogenesis Reveals Novel |
| | mesoderm | | Targets of Different WNT Signaling Pathways in the Somitic |
| | | | Mesoderm,” Dev. Biol. 258(1): 91-104 (2003) |

*Each of which are hereby incorporated by reference in their entirety.

According to some embodiments of the methods disclosed herein, contacting a subject or administering one or more of the compounds according to the present disclosure (e.g., a compound of Formula (I) and/or an immune checkpoint inhibitor) is effective to reduce at least one symptom of a disease or condition associated with a tumor and/or cancer having a dysregulated Wnt signaling pathway. For example, contacting a subject or administering one or more of the compounds according to the present disclosure may be effective to decrease a symptom of the disease or condition associated with the tumor and/or cancer (e.g., the size or a primary tumor, the presence of metastasis, the size of a metastasis) in a subject by at least 5%, 10%, 15%, 20%, 25%, 30%, 35%, 40%, 45%, 50%, 55%, 60%, 65%, 70%, 75%, 80%, 85%, 90%, 95%, 96%, 98%, 99%, or 100%.

As used herein, the term “survival” refers to a living patient and includes overall survival as well as progression-free survival. One-year and two-year survival rates refer to estimates of the proportion of subjects alive at 12 or 24 months. The term “overall survival” refers to the time from the start of treatment that the subject remains alive. The term “progression-free survival” refers to the time from treatment to the first day of disease progression.

The term “prolonging survival” refers to an increase in overall survival/or progression-free survival in treated subjects as compared to a control treatment protocol (e.g., sorafenib). Survival may be at least about one month, two months, three months, four months, five months, six months, 7 months, eight months, nine months, 10 months, 11 months, or at least about one year, at least about two years, at least about 3 years, at least about 4 years, at least about 5 years, at least about 6 years, at least about 7 years, at least about 8 years, at least about 9 years, at least about 10 years, or more after initiation of treatment or after initial diagnosis. It is monitored such as a year, or at least about four years, or at least about five years, or at least about ten years.

In some embodiments, said contacting or administering to the subject a compound of Formula (I) is effective to prolong the survival (i.e., overall survival and/or progression-free survival) of the selected subject to a greater extent than when the selected subject is treated with a control treatment protocol (e.g., sorafenib). In some embodiments, said contacting or administering to the subject a compound of Formula (I) and an immune checkpoint inhibitor is effective to prolong the survival (i.e., overall survival and/or progression-free survival) of the selected subject to a greater extent than when the selected subject is treated with a control treatment protocol (e.g., an immune checkpoint inhibitor monotherapy or sorafenib monotherapy).

### Methods of Treating Tumors and Cancers Having Cytoplasmic EZH2

As described herein, it has unexpectedly been found that WNTinib (i.e., a compound according to Formula (I) of the present disclosure) treatment elicits activation of EZH2 to selectively block transcription of Wnt targets. Thus, a further aspect of the present application relates to a method of treating a tumor. This method involves contacting a tumor comprising cytoplasmic EZH2 with a kinase inhibitor compound under conditions effective to treat the tumor.

Suitable tumors are described in detail supra. In some embodiments, the tumor is associated with a colorectal cancer; a gastric cancer; an endometrial cancer; a lung cancer; a liver cancer; a hepatocellular carcinoma; a hepatocellular adenoma; a hepatoblastoma; a melanoma; a bladder carcinoma; a pilomatrixoma; an ovarian cancer; a medulloblastoma; an adenocortical carcinoma; a pancreatic cancer; a NSCLC; a liver adenoma; a LIAD; a hepatoblastoma; or a cancer of the uterus, pancreas, prostate, stomach, bladder, anus, or esophagus.

Suitable compounds according to Formula (I) are described in detail supra.

In some embodiments, the kinase inhibitor compound is a compound of Formula (I) having the following structure:

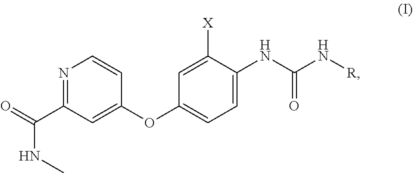

or a stereoisomer, pharmaceutically acceptable salt, oxide, or solvate thereof, wherein X is a halogen and R is a phenyl substituted with a perfluoroalkane. In some embodiments, the phenyl may be substituted with perflouroalkane in any one or more of the ortho, meta, and para positions. In some embodiments, the phenyl is substituted with a single perflouroalkane in the para position. In some embodiments, compounds of Formula (I) are as described herein, with the proviso that the compounds of Formula (I) do not include regorafenib. In some embodiments, compounds of Formula (I) are as described herein, with the proviso that R is a perfluoroalkane other than CF₃. In some embodiments of compounds of formula (I), R is a phenyl substituted with a perfluoroalkane comprising two or more carbon atoms.

In some embodiments, the method of treating a tumor comprising cytoplasmic EZH2 further involves contacting the tumor with an immune checkpoint inhibitor. Suitable immune checkpoint inhibitors are described in detail supra.

In some embodiments, cytoplasmic EZH2 is identified or detected in a immunohistochemistry, immunofluorescent, or western blot assay utilizing a phosphor-specific antibody able to selectively bind to pT367 EZH2.

In some embodiments, said contacting with a kinase inhibitor and/or an immune checkpoint inhibitor is carried out by administering the kinase inhibitor and/or the immune checkpoint inhibitor to a subject. The subject may be, e.g., a mammalian subject. In some embodiments, the subject is a human subject.

In some embodiments, said contacting is effective to reduce the amount of cytoplasmic EZH2 in the tumor. In some embodiments, the reduction in the amount of cytoplasmic EZH2 in the tumor is at least 10%, at least 20%, at least 30%, at least 40%, at least 50%, at least 60%, at least 70%, at least 80%, at least 90%, at least 95%, at least 96%, at least 97%, at least 98%, at least 99%, up to 100% (i.e., no detectable transcription and/or translation) or a reduction of at least 2-fold, at least 5-fold, at least 10-fold, at least 20-fold, at least 30-fold, at least 40-fold, at least 50-fold, or more relative to that observed in the absence contacting with the kinase inhibitor according to the present disclosure.

### EXAMPLES

The examples below are intended to exemplify the practice of embodiments of the disclosure but are by no means intended to limit the scope thereof.

### Example 1—General Chemical Methods

All solvents were purchased from Sigma-Aldrich and were used as received; anhydrous solvents were used for chemical reactions, and IPLC grade solvents were used for aqueous work-ups, recrystallizations, and chromatography. The palladium metal on solid support, used in hydrogenation reactions, was purchased from Sigma-Aldrich as 10% w/w on activated carbon (dry basis), with 50% w/w water added (Degussa type); designated in procedures as “5% w/w on activated carbon.” Other reagents were purchased from various vendors and were used as received. Reactions were run as described in the individual procedures using standard double manifold and syringe techniques. Glassware was dried by baking in an oven at 130° C. for 12 hours prior to use, or was flame-dried. The pH of aqueous solutions was estimated using pH paper. Vacuum filtrations were carried out using a house vacuum line (˜100 torr). In the individual procedures, the phrases “concentration under vacuum” and “concentrated to dryness” mean that solvent was removed on a rotary evaporator using a diaphragm pump (with an automatic vacuum regulator) and remaining traces of volatiles were removed on a high-vacuum (<1 torr) oil pump. Unless specified otherwise, the term “flask” refers to the round-bottomed variety. Reactions were monitored by TLC using EMD silica gel 60 F₂₅₄ (250 μm) glass-backed plates (visualized by UV fluorescence quenching and stained with basic KMnO₄ solution) and by liquid chromatography-tandem mass spectrometry (LC-MS). Analysis by reverse-phase LC-MS was carried out on a Waters Acquity I-Class UPLC system, with a C18 column (2.1×30 mm; 1.7 μm particle size), heated at 50° C., eluted at 0.6 mL/min, and using a 3 min linear gradient method with a mobile phase consisting of water/acetonitrile (0.1% v/v formic acid added to each): 95:5→1:99(0-2.5 min), then 1:99(2.5-3 min). Sample runs were monitored using alternating positive/negative electrospray ionization (50-1000 amu) and UV detection at 254 nm. Dimensions of plugs, pads, and columns for filtration or flash chromatography are reported as: ((diameter×length) cm). The 5¾ inch pipets (4 mL) used for filtration and micro scale flash chromatography were purchased from Fisher Scientific (product number 22-378-893). Automated preparative normal- and reverse-phase chromatography was carried out with an Interchim PuriFlash 450 purification system with a diode array detector (runs were monitored at 220-400 nm). Pre-packed silica gel cartridges (12, 25, and 40 g; 15 μm particle size) were employed for normal-phase (silica gel) chromatography, eluting at 20-30 mL/min. Preparative reverse-phase chromatography was carried out with an Agilent 1260 Infinity using a C18 column (30×100 mm; 5 μm particle size) with a multiwavelength detector, eluting at 40 mL/min with a pressure limit of 200 bar; crude samples were injected with an autosampler, typically in a 90:10 mixture of MeOH/DMSO (1.5 mL/injection). Carbon-decoupled ¹H NMR spectra were recorded at 400 MHz on a Bruker spectrometer and are reported in ppm using the residual solvent signal (dimethylsulfoxide-d6=2.50 ppm) as an internal standard. Data are reported as: {(shift), [(s=singlet, d=doublet, dd=doublet of doublets, ddd=doublet of a doublet of doublets, t=triplet, dt=doublet of triplets, q=quartet, quin=quintet, sext=sextet, sept=septet, m=multiplet, br=broad, ap=apparent), (J=coupling constant in Hz), (integration)]}. Proton-decoupled ¹³C NMR spectra were recorded at 100 MHz on a Bruker spectrometer and are reported in ppm using the residual solvent signal (dimethylsulfoxide-d₆=39.5 ppm) as an internal standard. Proton-decoupled ¹⁹F NMR spectra were recorded at 376 MHz on a Bruker spectrometer and are reported in ppm using added CFCl₃ (0.00 ppm) as an internal standard; compounds with only one signal were integrated relative to a known amount of the internal standard.

### Example 2—APS-8-89 (6): 4-(perfluoroethyl)aniline

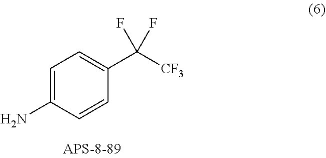 [SMILES: Nc1ccc(C(F)(F)C(F)(F)F)cc1]

A 150 mL sealable heavy-walled vessel was charged with tetrabutylammonium hydrogen sulfate (460 mg, 1.35 mmol), sodium bicarbonate (1.25 mg, 14.9 mmol), and water (35 mL). Sodium hydrosulfite (2.82 g, 16.2 mmol) was added in portions to the stirred solution over 1 min. The solution was diluted with methyl tert-butyl ether (MTBE; 15 mL) and then aniline (1.23 mL, 13.5 mmol) was added in a steady stream by syringe. To the biphasic mixture was added a solution of perfluoroethyl iodide (3.65 g, 14.8 mmol) and MTBE (10 mL) over 5 min by pipet. The solution was generated by bubbling perfluoroethyl iodide gas through MTBE cooled to 0° C. The headspace was purged with Ar, the vessel was sealed, and the reaction was stirred for 11 h protected from light. The biphasic mixture was transferred to a separatory funnel, the layers were separated, and the aqueous phase was extracted with Et₂O (50 mL). The organic extracts were pooled, washed with brine (25 mL), dried (Na₂SO₄), and filtered. The filtrate was concentrated under vacuum to leave a red oil, which was purified by silica gel chromatography (40 g cartridge), eluting at 40 mL/min and using a linear gradient of hexanes/CH₂Cl₂: 100:0→60:40 over 33 column volumes. Obtained 531 mg (19%) of the title compound as a yellow liquid: 1H NMR (400 MHz, DMSO-d₆) δ 7.25 (d, J=8.8 Hz, 2H), 6.66 (d, J=8.8 Hz, 2H), 5.84 (s, 2H); ¹⁹F NMR (376 MHz, DMSO-d₆) δ −83.8 (s, 3F), −111.0 (s, 2F); LC-MS (ESI+) m/z: [M+H]⁺ Calcd for C₈H₇F₅N, 212.0; Found 212.1.

### Example 3—APS-8-42-1 (8): 4-(perfluoropropan-2-yl)aniline

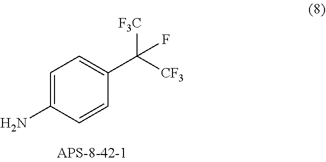 [SMILES: Nc1ccc(C(F)(C(F)(F)F)C(F)(F)F)cc1]

A 40 mL vial was charged with tetrabutylammonium hydrogen sulfate (298 mg, 0.878 mmol), sodium bicarbonate (811 mg, 9.65 mmol), and water (15 mL). Sodium hydrosulfite (1.68 g, 9.65 mmol) was added in portions to the stirred solution over 1 min. The solution was diluted with MTBE (15 mL) and then aniline (800 μL, 11.0 mmol) was added in a steady stream. To the biphasic mixture was added heptafluoro-2-iodopropane (1.37 mL, 9.63 mmol) over 5 min by syringe. The headspace was purged with Ar, the vial was sealed with a screwcap, and the reaction was stirred for 8 h protected from light. The biphasic mixture was transferred to a separatory funnel, the layers were separated, and the aqueous phase was extracted with Et₂O (25 mL). The organic extracts were pooled, washed with brine (25 mL), dried (Na₂SO₄), and filtered. The filtrate was concentrated under vacuum to leave a red oil, which was purified by silica gel chromatography (40 g cartridge), eluting at 40 mL/min and using a linear gradient of hexanes/CH₂Cl₂: 100:0→50:50 over 33 column volumes. Obtained 1.40 g (61%) of the title compound as a yellow liquid: ¹H NMR (400 MHz, DMSO-d₆) δ 7.23 (d, J=8.6 Hz, 2H), 6.68 (d, J=8.3 Hz, 2H), 5.75 (d, J=4.9 Hz, 2H); ¹⁹F NMR (376 MHz, DMSO-d₆) 6-75.0 (d, J=9.2 Hz, 6F), −179.7-−179.5 (m, 1F); LC-MS (ESI+) m/z: [M+H]⁺ Calcd for C₉H7F₇N, 262.0; Found 262.1.

### Example 4—APS-4-76 (67): 4-chloro-N-methylpicolinamide

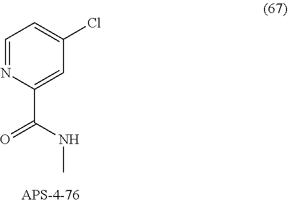

A flame-dried 250 mL flask, cooled under Ar, was charged with 4-chloropicolinic acid (10.0 g, 63.5 mmol), and THE (125 mL). The mixture was cooled to 0° C. and oxalyl chloride (6.70 mL, 79.2 mmol) was added dropwise over 5 min via syringe, followed by DMF (0.1 mL), which was added by syringe in one shot (CAUTION: rapid release of gas). After 30 min the reaction mixture was allowed to warm to room temperature and was stirred under a balloon of Ar for 15 h. The resulting brown solution was concentrated on a rotary-evaporator. A drying tube filled with KOH pellets was used to trap residual HCl. The remaining oil was concentrated to dryness from toluene (3×10 mL) and then was dried further under high vacuum to provide a solid. The crude 4-chloropicolinoyl chloride hydrochloride salt was placed under Ar and THF (50 mL) was added. The dark solution was cooled to 0° C. and methylamine (160 mL, 2.0 M solution in THF, 320 mmol) was added dropwise over 20 min via syringe. After 5 min the reaction was allowed to warm to room temperature and was stirred for 16 h. The reaction mixture was diluted with water (200 mL) and extracted with EtOAc (3×150 mL). The organic extracts were pooled, washed with water (100 mL) and brine (2×100 mL), dried (Na₂SO₄), and filtered. Concentration under vacuum gave ˜11 g of a red-brown oil, which was purified by silica gel chromatography (40 g cartridge), eluting at 30 mL/min, and using a linear gradient of hexanes/EtOAc: 100:0→0:100 over 30 column volumes. The appropriate fractions were pooled and concentrated to dryness. The remaining clear colorless oil (˜10 g) was dissolved in a mixture of hexanes/CH₂Cl₂ (4:1; 150 mL) and allowed to stand at −20° C. for 12 h. The resulting precipitate was isolated by vacuum filtration, washed with hexanes (2×30 mL), and air-dried to yield 8.90 g (82%) of the title compound as a white solid: ¹H NMR (400 MHz, DMSO-d₆) δ 8.85 (br ap d, J=3.4 Hz, 1H), 8.62 (dd, J=5.3, 0.6 Hz, 1H), 8.01 (dd, J=2.2, 0.6 Hz, 1H), 7.75 (dd, J=5.3, 2.2 Hz, 1H), 2.82 (d, J=4.9 Hz, 3H); ¹³C NMR (100 MHz, DMSO-d₆) δ 163.1, 151.8, 150.0, 144.5, 126.3, 121.8, 26.1; LC-MS (ESI+) m/z: [M+H]⁺ Calcd for C₇H₈ClN₂O, 171.0; Found 171.1.

### Example 5—APS-5-27 (5): 4-(4-amino-3-fluorophenoxy)-N-methylpicolinamide

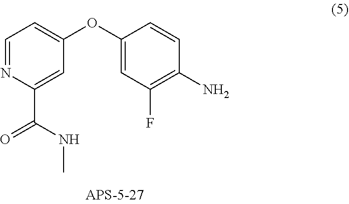 [SMILES: CNC(=O)c1cc(Oc2ccc(N)c(F)c2)ccn1]

An oven-dried two-necked 100 mL flask (equipped with an inlet adapter and septum), under Ar, was charged with 4-amino-3-fluorophenol (1.12 g, 8.81 mmol) and DMF (18 mL). To the stirred solution was added potassium tert-butoxide (978 mg, 8.72 mmol) in portions over 2 min. The resulting dark-purple mixture was stirred for 3 h, then 4-chloro-N-methylpicolinamide (1.06 g, 6.21 mmol) was added in one portion, and the reaction was heated at 90° C. for 10 h under a balloon of Ar. The reaction was allowed to cool to room temperature and then was poured into stirred ice-water (50 mL). Stirring was continued for 15 min and then the mixture was extracted with EtOAc (3×50 mL). The organic extracts were pooled; washed with 1 M KOH (3×50 mL), water (50 mL), and brine (2×50 mL); dried (Na₂SO₄); and filtered. Concentration under vacuum gave a brown solid, which was purified by silica gel chromatography (40 g cartridge), eluting at 30 mL/min and using a linear gradient of hexanes/EtOAc: 100:0→0:100 over 38 column volumes. Obtained 882 mg (54%) of the title compound as a light-brown solid: ¹H NMR (400 MHz, DMSO-d₆) δ 8.74 (br q, J=4.6 Hz, 1H), 8.47 (d, J=5.6 Hz, 1H), 7.35 (d, J=2.5 Hz, 1H), 7.09 (dd, J=5.6, 2.7 Hz, 1H), 7.01 (dd, J=11.9, 2.6 Hz, 1H), 6.81-6.89 (m, 1H), 6.76-6.80 (m, 1H), 5.22 (br s, 2H), 2.78 (d, J=4.9 Hz, 3H); ¹⁹F NMR (376 MHz, DMSO-d₆) δ-130.7 (s, 1F); LC-MS (ESI+) m/z: [M+H]⁺ Calcd for C₁₃H₁₃FN₃O₂ 262.1; Found 262.1.

### Example 6—WNTinib1: APS-8-100-2 (40): 4-(3-fluoro-4-(3-(4-(perfluoroethyl)phenyl)ureido)phenoxy)-N-methylpicolinamide

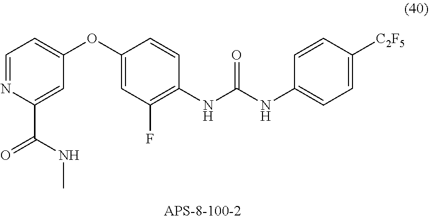

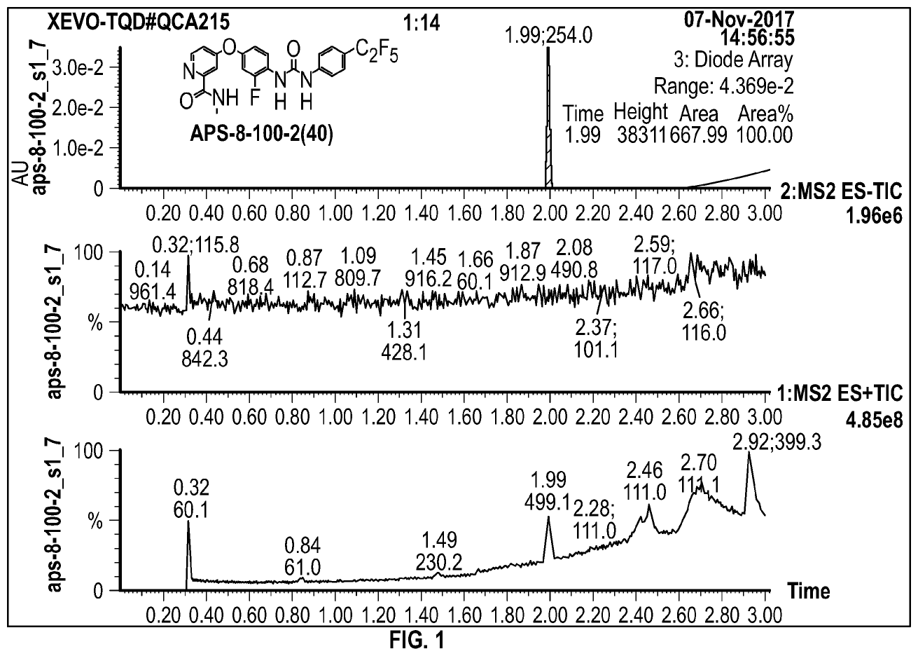

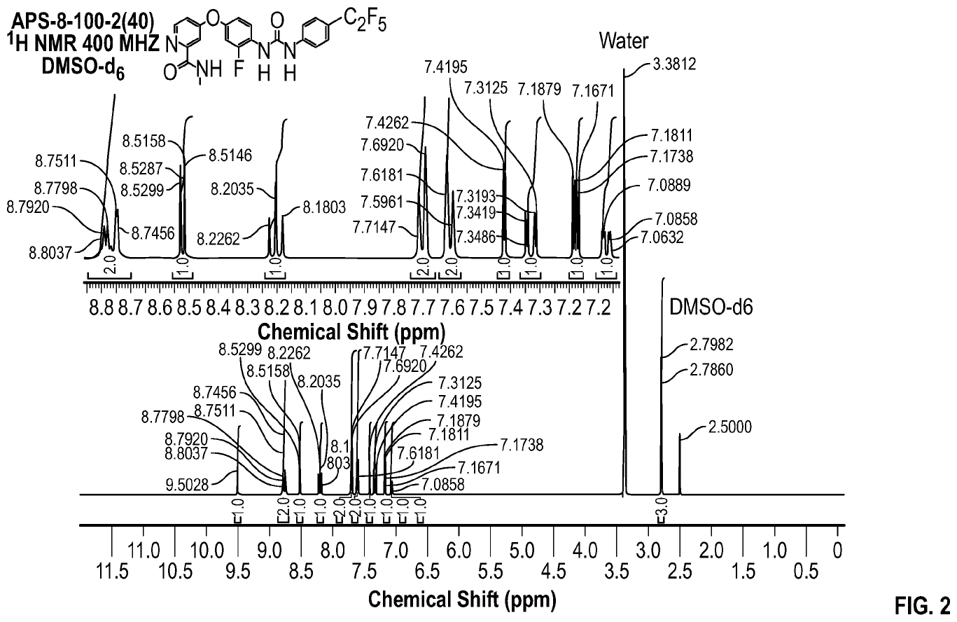

A solution of 4-(perfluoroethyl)aniline (6; 87.0 mg, 0.412 mmol) and CH₂Cl₂ (1 mL) was added to a 0° C. solution of CDI (70.0 mg, 0.432 mmol) and CH₂Cl₂ (1 mL) dropwise over 1 min. The solution was stirred at 0° C. for 1 h and then at room temperature for 24 h. To the acyl imidazole solution was added 5 (107 mg, 0.410 mmol) in one portion and stirring was continued for 12 h. Purification by silica gel chromatography (25 g cartridge), eluting at 25 mL/min and using a linear gradient of CH₂Cl₂/EtOAc: 100:0→50:50 over 25 column volumes, provided 107 mg (52%) of the title compound as a white solid: ¹H NMR (400 MHz, DMSO-d₆) δ 9.50 (s, 1H), 8.70-8.85 (m, 2H), 8.52 (dd, J=5.6, 0.5 Hz, 1H), 8.20 (t, J=9.2 Hz, 1H), 7.66-7.75 (m, 2H), 7.57-7.64 (m, 2H), 7.40-7.44 (m, 1H), 7.33 (dd, J=11.7, 2.7 Hz, 1H), 7.18 (dd, J=5.6, 2.7 Hz, 1H), 7.07 (ddd, J=8.9, 2.7, 1.1 Hz, 1H), 2.79 (d, J=4.9 Hz, 3H); ¹⁹F NMR (376 MHz, DMSO-d₆) δ-83.8-−83.7 (m, 3F), −112.5-−112.1 (m, 2F), −124.4 (s, 1F); LC-MS (ESI+) m/z: [M+H]⁺ Calcd for C₂₂H₁₇F₆N₄O₃ 499.1; Found 499.1. See FIG. 1 and FIG. 2.

### Example 7—WNTinib2: APS-8-50-2 (36): 4-(3-fluoro-4-(3-(4-(perfluoropropan-2-yl)phenyl)ureido)phenoxy)-N-methylpicolinamide

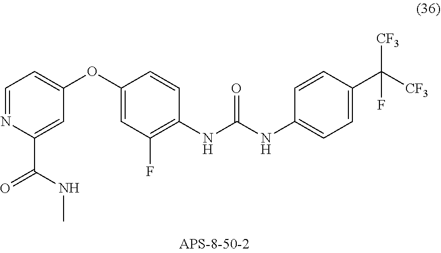 [SMILES: CNC(=O)c1cc(Oc2ccc(NC(=O)Nc3ccc(C(F)(C(F)(F)F)C(F)(F)F)cc3)c(F)c2)ccn1]

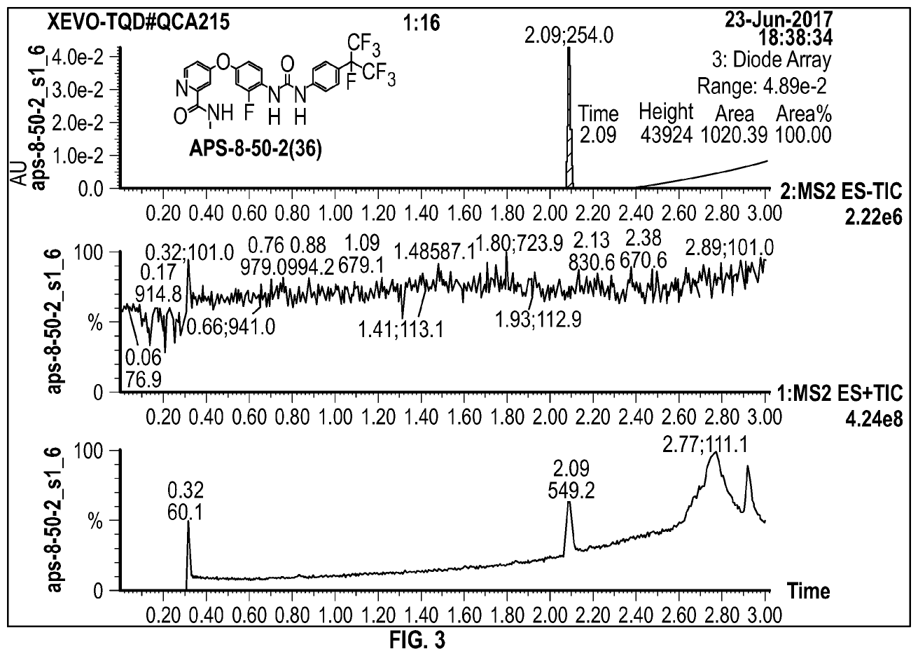

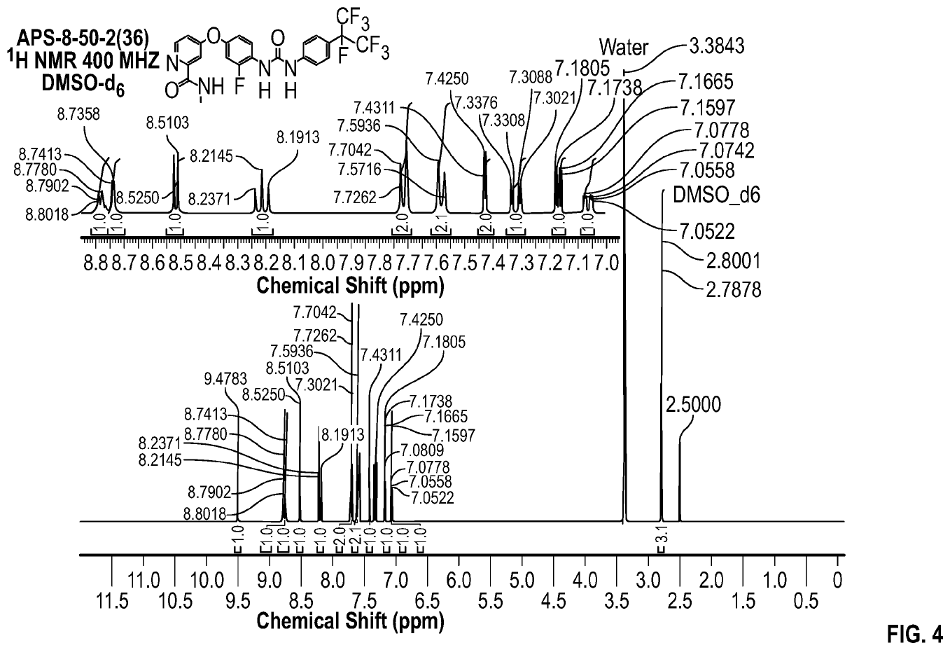

A solution of 4-(perfluoropropan-2-yl)aniline (8; 107 mg, 0.410 mmol) and CH₂Cl₂ (1 mL) was added to a 0° C. solution of CDI (70.0 mg, 0.432 mmol) and CH₂Cl₂ (1 mL) dropwise over 1 min. The solution was stirred at 0° C. for 1 h and then at room temperature for 24 h. To the acyl imidazole solution was added 5 (107 mg, 0.410 mmol) in one portion and stirring was continued for 12 h. Purification by silica gel chromatography (25 g cartridge), eluting at 25 mL/min and using a linear gradient of CH₂Cl₂/EtOAc: 100:0→65:35 over 23 column volumes, provided 145 mg (64%) of the title compound as a white solid: ¹H NMR (400 MHz, DMSO-d₆) δ 9.48 (s, 1H), 8.78 (q, J=4.8 Hz, 1H), 8.74 (d, J=2.2 Hz, 1H), 8.52 (d, J=5.9 Hz, 1H), 8.21 (t, J=9.2 Hz, 1H), 7.72 (d, J=8.8 Hz, 2H), 7.58 (d, J=8.8 Hz, 2H), 7.43 (d, J=2.4 Hz, 1H), 7.32 (dd, J=11.5, 2.7 Hz, 1H), 7.17 (dd, J=5.6, 2.7 Hz, 1H), 7.07 (ddd, J=8.9, 2.6, 1.2 Hz, 1H), 2.79 (d, J=4.9 Hz, 3H); ¹⁹F NMR (376 MHz, DMSO-d₆) δ-74.9 (d, J=6.9 Hz, 6F), −124.6-−124.5 (m, 1F), −180.8-−180.7 (m, 1F); LC-MS (ESI+) m/z: [M+H]⁺ Calcd for C₂₃H₁₇F₈N₄O₃ 549.1; Found 549.2. See FIG. 3 and FIG. 4.

### Materials and Methods for Examples 8-11

### Hydrodynamic Tail-Vein Injections

Vectors were specifically delivered into hepatocytes by injecting a solution of sterile saline (0.9%) containing 10 μg of transposon vectors (pT3-EF1α-MYC (Addgene #92046), pT3-EF1α-Tert, pT3-N90-CTNNB1 (Addgene #31785)), 10 μg CRISPR/Cas9 vectors (px330-sgKmt2c, px330-sgPten), and 2.5 μg of the transposon-encoding vector SB13-Luc according to each specific condition. A volume equivalent to 10% of mouse body weight was injected through the lateral tail vein using a 3 mL syringe with a 26-gauge needle (Molina-Sanchez et al., “Cooperation Between Distinct Cancer Driver Genes Underlies Intertumor Heterogeneity in Hepatocellular Carcinoma,” Gastroenterology 159(6):2203-2220 (2020), which is hereby incorporated by reference in its entirety).

### Tumor Organoid Derivation and Culturing

Liver tumors were minced and digested in sterile digestion media (PBS, 0.125 mg/mL collagenase from Clostridium histolyticum, 0.125 mg/mL dispase II, and 0.1 mg/mL DNaseI) for two hours at 37° C. Tumor dissociate was strained through a 70 μm strainer and washed with basal media (Advanced DMEM/F-12, 1% glutamine, 1% penicillin/streptomycin, 10 mM HEPES). Cells were counted, washed, resuspended at 50,000 cells per 50 μL Matrigel (Corning), and plated in 24 well plates. Cell dissociate was cultured in murine tumor organoid media (basal media, 1:50 B27, 1 mM N-acetylcysteine, 10% Rspo1-conditioned media, 10 mM nicotinamide, 10 nM recombinant human [Leu¹⁵]-gastrin I, 50 ng/mL recombinant mouse EGF, 100 ng/mL recombinant human FGF10, and 50 ng/mL recombinant human HGF) until organoids formed (Broutier et al., “Culture and Establishment of Self-Renewing Human and Mouse Adult Liver and Pancreas 3D Organoids and their Genetic Manipulation,” Nat. Protoc. 1:1724-1743 (2016), which is hereby incorporated by reference in its entirety). To passage, organoids were taken out of Matrigel in basal media, spun down at 300 g for 5 minutes at 4° C., mechanically broken by passing through a 21-gauge needle, washed in basal media, and plated in Matrigel. Patient-derived organoids were cultured in human tumor organoid media (basal media, 1:50 B27 no vitamin A, 1:100 N2, 1 mM N-acetylcysteine, 10% Rspo1-conditioned media, 10 mM nicotinamide, 10 nM recombinant human [Leu¹⁵]-gastrin I, 50 ng/mL recombinant human EGF, 100 ng/mL recombinant human FGF10, 25 ng/mL recombinant human HGF, 10 μM forskolin, and 5 μM A83-01) and passaged as above. For all tumor organoids, media was changed twice a week, and organoids were split upon attainment of dense cultures. Hematoxylin and eosin staining was performed as published (Broutier et al., “Culture and Establishment of Self-Renewing Human and Mouse Adult Liver and Pancreas 3D Organoids and their Genetic Manipulation,” Nat. Protoc. 1:1724-1743 (2016), which is hereby incorporated by reference in its entirety).

### Adherent Cell Line Culturing

All cell lines were grown in complete RPMI (10% FBS, 1% glutamine, 1% penicillin/streptomycin). Media was changed twice a week, and cells were split upon attainment of dense cultures.

### Drug Toxicity Assays

For assays in organoid lines, 96 well plates were first coated with a 50:50 solution of basal media:Matrigel (35 μL/well), which polymerized for 15 minutes at 37° C. Tumor organoids were taken out of Matrigel, broken, and washed. Tumor organoids were seeded at 1,000 cells per well and were treated the following day with serial dilutions (in the case of IC₅₀ curves) or single doses (in the case of the initial screen) of drugs in technical triplicate. Final DMSO concentrations were kept below 0.5%. For adherent lines, cells were seeded at 5,000 cells per well in uncoated 96 well plates. Murine passaged human primary hepatocytes (mpPHH) were isolated as published and seeded at 40,000 cells per well in collagen-coated 96 well plates (Michailidis et al., “Expansion, in Vivo-Ex Vivo Cycling, and Genetic Manipulation of Primary Human Hepatocytes,” Proc. Natl. Acad. Sci. USA 117(3):1678-1688 (2020), which is hereby incorporated by reference in its entirety). Viability was measured three days post drug administration with either luminescence from CellTiter-Glo or absorbance at 560 and 590 nm from resazurin. Viability data was analyzed by normalizing individual drug-treated well values to DMSO-treated wells. BLISS scores were computed using SynergyFinder (Ianevski et al., “SyngeryFinder: A Web Application for Analyzing Drug Combination Dose-Response Matrix Data,” Bioinformatics 33(15):2413-2415 (2017), which is hereby incorporated by reference in its entirety).

### Human Primary HCC Cell Line Generation and Genotyping

Biopsies were minced and digested in sterile digestion media (PBS, 1 mg/mL collagenase IV) for 90 minutes at 37° C. Tumor dissociate was strained though a 70 μm strainer and washed with complete RPMI (20% FBS, 1% glutamine, 1% penicillin/streptomycin). Dissociate was counted, and 100,000 cells were plated on collagen-coated 35 mm plates in primary HCC media (complete RPMI, 40 ng/mL recombinant human EGF, 10 μM Y-27632, and 5 μM A83-01). Media was changed twice a week until the formation of tumor colonies, which were manually picked for further propagation. For CTNNB1 genotyping, genomic DNA was isolated from cell lines (Qiagen). The target genomic region was amplified by PCR using primers listed in Table 5 below. PCR was performed with Herculase II Fusion DNA polymerase (Agilent) according to manufacturer's instructions using 200 ng of genomic DNA as a template. PCR conditions were: 95°×2 minutes, 95°-0:20→58°-0:20→72°-0:30×34 cycles, 72° C.×3 minutes. The PCR product was column purified (Qiagen) and submitted for MiSeq (Macrogen).

TABLE 5
Oligonucleotide Sequences

| | | SEQ |
|---|---|---|
| | | ID |
| Oligonucleotide | Sequence | NO: |
| CTNNB1-MiSeq-F | TCAATGGGTCATATCACAGATTCT | 12 |
| CTNNB1-MiSeq-R | TCCTCTTCCTCAGGATTGCC | 13 |
| Axin2-qPCR-F | TGACTCTCCTTCCAGATCCCA | 14 |
| Axin2-qPCR-R | TGCCCACACTAGGCTGACA | 15 |
| Jun-qPCR-F | CCTTCTACGACGATGCCCTC | 16 |
| Jun-qPCR-R | GGTTCAAGGTCATGCTCTGTTT | 17 |
| Wnt16-qPCR-F | GCAGGCTGTCGCCAAGTTA | 18 |
| Wnt16-qPCR-R | GTCTGCCTCTGGTCTTTTTCTC | 19 |
| Lef1-qPCR-F | TGTTTATCCCATCACGGGTGG | 20 |
| Lef1-qPCR-R | CATGGAAGTGTCGCCTGACAG | 21 |
| Gapdh-qPCR-F | TGGATTTGGACGCATTGGTC | 22 |
| Gapdh-qPCR-R | TTTGCACTGGTACGTGTTGAT | 23 |
| Actb-qPCR-F | GGCTGTATTCCCCTCCATCG | 24 |
| Actb-qPCR-R | CCAGTTGGTAACAATGCCATGT | 25 |
| Ezh2-shRNA-1 | CCGGCGGCTCCTCTAACCATGTTT | 26 |
| | ACTCGAGTAAACATGGTTAGAGGA | |
| | GCCGTTTTTG | |
| Ezh2-shRNA-2 | CCGGGCACAAGTCATCCCGTTAAA | 27 |
| | GCTCGAGCTTTAACGGGATGACTT | |
| | GTGCTTTTTG | |
| shRNA-Ctrl | CCGGGCGCGATAGCGCTAATAATT | 28 |
| | TCTCGAGAAATTATTAGCGCTATC | |
| | GCGCTTTTT | |

### Kinome Profiling

WNTinib's inhibitory profile was performed using Eurofins' (DiscoverX) KINOMVEscan platform. Inhibition trees were visualized using KinMap (Eid et al., “KinMap: A Web-Based Tool for Interactive Navigation through Human Kinome Data,” BMC 10 Bioinformatics 18(1):16 (2017), which is hereby incorporated by reference in its entirety). For regorafenib and sorafenib, publicly available data was used to visualize inhibition trees (Uitdehaag et al., “Comparison of the Cancer Gene Targeting and Biochemical Selectivities of All Targeted Kinase Inhibitors Approved for Clinical Use,” Plos ONE 9(3):e92146 (2014) and Davis et al., “Comprehensive Analysis of Kinase Inhibitor Selectivity,” Nat. Biotechnol. 29(11):1046-1051 (2011), which are hereby incorporated by reference in their entirety).

### Phosphoproteomics

Lysate Preparation

Tumor organoids were grown in 24 well plates, and after drug treatment for 24 hours, were collected in Cell Recovery Solution (Corning). Tumor organoids were left to rotate at 4° C. for one hour in order to dissolve the Matrigel. Tumor organoids were spun down at 300 g at 4° C. for 5 minutes. 10 million cells were counted, lysed in urea buffer (8 M urea, 50 mM Tris pH 8.2) for 10 minutes, sonicated, and centrifuged at maximum speed for 10 minutes at 4° C. The cleared lysate's concentration was measured using the BCA protein assay. The reduced and alkylated proteins in the lysate were digested overnight with LysC, followed by another overnight digestion with trypsin at the enzyme to protein ratio of 1:25 (mg). Tryptic digestion was stopped with 1% TFA, and the digested peptides were desalted using a HLB 6 cc cartridge (Waters). The cartridge was conditioned with 1 mL ACN, followed by 1 mL 0.5% acetic acid and samples were loaded onto the cartridge. The cartridge was washed with 1 mL 0.5% acetic acid and eluted with 1 ml 65% ACN in 0.5% acetic acid. The elution was performed once more by reloading the eluate into the cartridge. Phosphoenrichment was performed with the titansphere (TiO2) method. Input control from the desalted eluate was collected for analysis prior to enrichment, and the remaining eluate was brought to a final concentration of 40% ACN and 6% TFA. Samples were sequentially incubated with 2 mg of titansphere per 1 mg of peptide twice, and the beads were loaded onto C8 Stage-Tips. The beads were washed with 10% ACN, 40% ACN, and 60% ACN, all with 6% TFA, and eluted sequentially with 5% ammonia water and 10% ammonia water with 25% ACN. Phosphoenriched eluates were desalted, labelled with TMT6plex in 100 mM TEAB and quenched with 1M NH₄HCO₂. The pooled labelled samples were desalted and fractionated with HpH C18 10 μm resin as above.

Mass Spectrometry

Analysis was performed using the Easy nLC1000 (Thermo) chromatography system coupled with Orbitrap Fusion (Thermo). Each sample was separated into 90 minute gradients (0.1% FA, 99.9% ACN with 0.1% FA) using 50 cm×75 μm ID Easy-Spray column (C-18, 2 μm particles). The following acquisition parameters were applied: data dependent mode in a speed mode −3 sec cycle, OT-MS resolution 60K, 4e5 ions, OT-MS/MS 7.5K, AGC target of 5e4 and HCD fragmentation at 40% collision energy, isolation window 1 m/z.

Data Processing

Peak lists were generated in Proteome Discoverer 2.3 software using Mascot with forward/decoy mouse Uniprot database searches with the following parameters: precursor mass tolerance MS 20 ppm, MS/MS 0.06 Da, 3 missed cleavages, static modifications: carbamidomethyl (C), TMT 6plex, variable modifications: acetyl (N-terminal protein), deamidated (NQ), oxidation (M), phospho (STY) with 2 levels of false discovery rate (FDR): strict=FDR 1%, medium=FDR 5%.

Computational Analysis

Quantified protein abundances were imported into the R environment to facilitate data analysis and visualization (https://www.R-project.org/). Only proteins with at least two quantifying peptide counts were used for downstream analysis. This filtering was not applied to individual site data. Total phosphoproteins and sites were normalized by first adjusting the summed abundance in each sample to be the same as the averaged value from all the samples, followed by variance stabilization normalization. Differential expression was determined by using estimates of variance-mean dependence (DESeq2) with a Benjamini-Hochberg FDR correction. Phosphoproteins significantly (FDR<0.05) modulated by WNTinib were used to perform a STRING interactome analysis with parameters: highest confidence interaction score and 3 kmeans clusters. Phosphoproteins belonging to individual clusters were used for a gProfiler analysis to identify enriched reactome pathways, which also identified leading phosphoproteins. To compute kinase-substrate relationships, missing values in raw data were imputated with PhosR (for data available in at least 1 replicate per condition) (Kim et al., “PhosR Enables Processing and Functional Analysis of Phosphoproteomic Data,” Cell Rep. 34(8):108771 (2021), which is hereby incorporated by reference in its entirety). Sites were median-centered, with additional normalization to subtract the effect of non-changing phosphorylated sites. Ratios of samples treated with WNTinib against DMSO were taken for combined kinase-substrate score calculation. Ratios were further filtered based on adjusted p-value <0.05 (ANOVA test) to identify top kinases responsible for phosphorylation sites. Kinase-substrate predictions were also computed by downloading predicted kinase scores from PhosphoNet, followed by rank ordering of score averages for individual kinases. All phosphoproteomic data files are submitted to JPOST Repository with the identifier (JPST001111; PXID: PXD024958; reviewer access key 1167).

### RNA Sequencing

RNA Preparation

Tumor organoids were grown in 24 well plates, and after drug treatment for 24 hours, were collected in Trizol. RNA was extracted using standard procedures and quality was assessed with a bioanalyzer. Libraries were built using the NEBNext Ultra II total RNA kit and sequenced with a HiSeq X (PE150).

Computational Analysis

Raw FASTQ files were pseudoaligned to a reference transcriptome generated from the mouse genome GRCm38 and transcript abundances were estimated using Kallisto (v0.46.1). The quality of the transcriptomic data was checked and reported with FastQC and MultiQC. Transcripts were mapped to gene symbols with biomaRt (v2.46.3) using the Ensembl 96 release, and gene-level read counts were obtained by aggregating all transcripts per gene. Differentially expressed genes were identified by DESeq2 (v1.30.0) and analyses were done on log₂-transformed data. Genes with a q-value <0.05 (p-value adjusted according to the Benjamini-Hochberg multiple comparison adjustment) were included in following analysis. Pathway enrichment analysis was done with gProfiler and GSEA. All RNAseq data files are submitted to the NCBI Gene Expression Omnibus database (http://www.ncbi.nlm.nih.gov/geo) with the accession number (GSE169444; reviewer token: yvsxakqmnvshtih).

### Quantitative PCR

1 μg of Trizol-purified RNA was used for cDNA preparation using the Applied Biosystems High-Capacity cDNA kit. qPCR was performed using Bio-Rad SYBR green master mix on a QuantStudio 5 thermal cycler (ThermoFisher). Primer sequences can be found in Table 5.

### Western Blotting

Tumor organoids were grown in 24 well plates and were collected in Cell Recovery Solution (Corning) after drug treatment. Tumor organoids were left to rotate at 4° C. for one hour in order to dissolve the Matrigel. Tumor organoids were spun down at 300 g at 4° C. for 5 minutes. Pellets were lysed in 2× Laemmli buffer (+4% 2-mercaptoethanol) by heating at 95° C. for 10 minutes followed by 5 seconds of sonication. SDS-PAGE was run using standard procedures. Antibody information can be found in Table 6.

TABLE 6
Antibody Information

| Antibody | Manufacturer | Catalog ID |
|---|---|---|
| Actb | Abcam | ab8227 |
| ATF-2 (D4L2X) XP | Cell Signaling | 35031 |
| ATF2 Phospho T71 | Abcam | ab32019 |
| B-Tubulin | Cell Signaling | 2146 |
| Ezh2 (D2C9) XP | Cell Signaling | 5246 |
| Ezh2 Phospho Thr367 | MyBioSource | MBS9429020 |
| GAPDH (D16H11) XP | Cell Signaling | 5174 |
| Histone H3 | Abcam | ab1791 |
| MKK6 | Cell Signaling | 9264 |
| Vinculin | Cell Signaling | 13901 |
| H3K27me3 | Active Motif | 39155 |

### Cellular Fractionation

Tumor organoids were grown in 24 well plates and were collected in Cell Recovery Solution (Corning) after drug treatment. Tumor organoids were left to rotate at 4° C. for one hour in order to dissolve the Matrigel. Tumor organoids were spun down at 300 g at 4° C. for 5 minutes, counted, and 1 million cells were aliquoted per condition. Cells were spun down again at 500 g, 4° C., for 5 minutes in ultra-low attachment 1.5 mL Eppendorf tubes. Cells were resuspended in 100 μL of lysis buffer 1 (50 mM HEPES-KOH, pH 7.5, 140 mM NaCl, 1 mM EDTA, 10% glycerol, 0.5% NP-40, 0.25% Triton X-100, lx protease/phosphatase inhibitors) and agitated at 4° C. for 10 minutes. Lysate was spun at 1,350 g, 4° C., for 5 minutes. The supernatant was saved for the cytoplasmic fraction. The pellet was resuspended in 100 μL of lysis buffer 2 (10 mM Tris-HCl, pH 8.0, 200 mM NaCl, 1 mM EDTA, 0.5 mM EGTA, lx protease/phosphatase inhibitors) and agitated at room temperature for 10 minutes. The supernatant was saved for the nuclear fraction. The pellet was resuspended in 35 μL of lysis buffer 3 (10 mM Tris-HCl, pH 8.0, 100 mM NaCl, 1 mM EDTA, 0.5 mM EGTA, 0.1% Na-Deoxycholate, 0.5% N-lauroylsarcosine, lx protease/phosphatase inhibitors) and agitated at 4° C. for 10 minutes. 1/10 volume of 10% Triton X-100 was added to the lysate, which was spun down at 20,000 g, 4° C., for 10 minutes. Supernatant was taken for chromatin fraction. Lysates were diluted 1:1 with 2× laemmli buffer (+4% 2-mercaptoethanol) and heated at 95° C. for 10 minutes followed by five seconds of sonication. SDS-PAGE was performed using standard procedures.

### Lentivirus Production and Infection

For MKK6 overexpression, FLAG-MKK6(S207E, T211E)-pcw107 (Addgene #64625) was used. For EZH2 depletion, MISSION shRNA constructs were used (Sigma, TRC2 series). shRNA sequences can be found in Table 5. To generate lentivirus, HEK293T cells were placed in antibiotic-free DMEM (Gibco) supplemented with 10% fetal bovine serum. Lentiviral packaging plasmids (pCMV-VSVG, pCMV-Δ8.9) and plasmids of interest were transfected into cells using Lipofectamine 3000 (ThermoFisher). Virus-containing supernatant was harvested 48 and 72 hours after transfection, and viral particles were concentrated with Lenti-X Concentrator according to the manufacturer's instructions (Takara Bio). Lenti-X GoStix Plus were utilized to determine virus titer (Takara Bio). For lentiviral infection of SNU398 cells, lentivirus was added at a multiplicity of infection (MOI) of 3 along with 8 μg/ml of Polybrene Infection/Transfection Reagent (EMD Millipore). Puromycin was added at a concentration of 2 μg/ml 48 hours after infection. Cells were incubated with puromycin for 4 days until all cells in an uninfected control well died. After infection and selection, stable overexpression was confirmed by western blotting. For lentiviral infection of organoids, organoids were collected in Cell Recovery Solution (Corning) and were left to rotate at 4° C. for one hour in order to dissolve the matrigel. Organoids were spun down at 300 g, 4° C., for 5 minutes. Organoids were dissociated into single cells with TrypLE (Gibco) by rotating at room temperature for 5 minutes. After rotation, an equivalent volume of basal media was added, and cells were centrifuged at 300 g, 4° C., for 5 minutes. Cells were resuspended in transduction media (murine tumor organoid media+10 μM Y-27632, 3 μM CHIR99021), and TransDUX MAX (Takara Bio) reagents were added according to manufacturer's instructions. Cell suspensions were distributed into ultra low-attachment 24-well plates (Corning), and lentiviruses were added at a MOI of 3. Parafilm-wrapped plates were then spun at 600 g for 1 hour at 32° C. Following centrifugation, cells were incubated at 37° C. for 4-6 hours. Infected cells were collected in 15 ml conical tubes, centrifuged at 300 g for 5 minutes at 4° C., and redistributed into new 24-well plates in Matrigel. Organoids were grown in transduction media, and 2 μg/ml of puromycin was added 48 hours after infection. Organoids were incubated with puromycin for 4 days until all organoids in an uninfected control well died. After infection and selection, stable overexpression or depletion was confirmed by western blotting or qPCR.

### Cellular Thermal Shift Assay (CETSA)

SNU398 cells were trypsinized, centrifuged at 300 g for 5 minutes, washed once in PBS, and centrifuged at 300 g for 5 minutes. 1.3 million cells/condition were resuspended in CETSA Buffer (50 mM HEPES pH 7.5, 10 mM MgCl₂, lx protease/phosphatase inhibitors). Samples were centrifuged at maximum speed at 4° C. for 20 minutes, and supernatant was collected. The supernatant was aliquoted into PCR tubes, and 10 μM of test compounds or DMSO were added to individual tubes for a 30-minute incubation period. After drug incubation, samples were heated to a gradient of temperatures for 3 minutes and cooled to 37° C. for 3 minutes. After heat treatment, samples were centrifuged at max speed at 4° C. for 10 minutes, and supernatants were transferred to new tubes. Lysates were diluted 1:1 with 2× laemmli buffer (+4% 2-mercaptoethanol), vortexed, heated at 70° C. for 10 minutes, and subsequently run using SDS-PAGE.

### NanoBRET™

For NanoBRET™ dose response experiments, SNU398 cells were transfected with a MAPK14-NanoLuc© Fusion Vector according to the Promega NanoBRET™ TE Intracellular Kinase Assay protocol. Following overnight transfection, cells were trypsinized and redistributed on a white 96-well plate (20,000 cells per well). Cells were incubated with 1 μM of K-4 Tracer and sorafenib or WNTinib at doses ranging from 0.02→20 μM for two hours. After drug incubation, plates were equilibrated to room temperature for 15 minutes, and NanoBRET™ Nano-Glo© Substrate and Extracellular NanoLuc© Inhibitor were added to wells. Bioluminescent donor (NanoLuc© luciferase) and fluorescent acceptor (Tracer) emission were measured on a GloMax plate reader at 450 and 600 nm, respectively. BRET ratio is reported as acceptor signal divided by the donor signal for each well. Conditions were evaluated in technical triplicates for each experiment, and experiments were repeated in biological duplicates. For washout experiments, the Promega NanoBRET™ TE Intracellular Kinase Assay protocol was modified so that 150,000 cells were plated per well instead of 20,000. After transfection, cells were incubated with sorafenib or WNTinib at either 1 or 10 μM or with DMSO. Following a 2 hour drug incubation, cells were centrifuged at 300 g for 5 minutes, and the supernatant was aspirated to remove drug from media. After two washes in OptiMEM (Gibco), cells were resuspended in OptiMEM and plated on a white 96-well plate. K-4 Tracer, NanoBRET™ Nano-Glo© Substrate, and Extracellular NanoLuc© Inhibitor were added to cells immediately before recording donor and acceptor signal. The plate was continuously read on a GloMax plate reader for 2 hours to record BRET signal from the association of the K-4 Tracer with the MAPK14-NanoLuc© Fusion Vector. Conditions were evaluated in technical triplicates, and experiments were repeated in biological duplicates.

### Mice

6-8-week-old C57BL/6J, BALB/c, and BALB/c nude female mice were obtained from Envigo. All mouse studies were approved by the Icahn School of Medicine at Mount Sinai (ISMMS) Institutional Animal Care and Use Committee (protocol number 2018-0013). Mice were maintained under pathogen-free conditions, and food and water were provided ad libitum. All animals were examined prior to initiation of studies and were acclimated to the laboratory environment for one week prior to use.

### In Vivo Studies

Drug Preparation

Solid stocks of drugs were first dissolved in a 1:1 solution of Kolliphor EL:ethanol (Sigma and frozen at −80° C. Drugs were diluted with 4 parts water before oral gavage (200 μL/mouse).

Dose Escalation Studies

Mice were randomized into treatment groups, and daily oral dosing began one week after mice arrived at ISMMS. Body weight was recorded daily, and animals were monitored for any signs of sickness throughout the treatment period.

Allograft Studies

Tumor organoids were grown in 24 well plates, taken out of Matrigel in basal media, spun down at 300 g for 5 minutes at 4° C., mechanically broken by passing through a 21-gauge needle, and counted. 300,000 cells per allograft were prepared in 8 mg/mL Matrigel. 6-8-week-old C57BL/6J mice were shaved the day prior to injection. A 26^{5/8}-gauge needle was used to implant organoids into the flank in 100 μL Matrigel. Tumor allografts were measured daily with calipers, and upon an average volumetric measurement of 100 mm³, animals were randomized and drug dosing was started. Drugs were dosed daily at 30 mg/kg via oral gavage. Tumor allografts were also measured daily.

HDTV Study

A sterile 0.9% NaCl solution/plasmid mix was prepared containing DNA—12 μg of pT3-EF1α-MYC-IRES-luciferase-OS (MYC-lucOS), 10 μg of pT3-N90-CTNNB1 (CTNNB1), and a 4:1 ratio of transposon to SB13 transposase-encoding plasmid. A volume equivalent to 10% of mouse body weight was injected through the tail vein using a 3 mL syringe with a 26-gauge needle (11). Animals were monitored for 6 days, and bioluminescent signal from tumors was visualized using an IVIS Spectrum system. Anesthetized mice were imaged 5 minutes after intraperitoneal injection of 100 mg/kg D-luciferin (Thermo Fisher Scientific). IVIS Living Image software was used to quantify luciferase signal, which was normalized by subtracting background signal. Mice were randomized into treatment groups, where each group had an equivalent average luciferase signal. Oral drug treatment began in randomized arms seven days after plasmid injection. Kinase inhibitors were prepared as stated above, and WNTinib and sorafenib were dosed at concentrations of 20 mg/kg and 30 mg/kg, respectively. Drugs were administered following a 5 days on/2 days off treatment schedule. Body weights were recorded daily, and animals were monitored for signs of sickness, tumor palpability, and death. Sick animals were sacrificed according to the ISMMS IACUC guidelines.

### Pharmacokinetics

All animal experiments were carried out in accordance with ethical guidelines and approved by the Institutional Animal Care and Use Committee (IACUC), Biological Resource Center (BRC) A*STAR. BALB/c mice were purchased from InVivos, Singapore and fed with a standard laboratory diet and distilled water ad libitum. The animals were kept on a 12-hour light/dark cycle at 22±2° C. in individually ventilated caging systems with 50-65% humidity in the Biological Resource Centre, A*Star, Singapore. For single dose oral pharmacokinetic studies, female BALB/c mice (n=8, 6-8 weeks old) were used. The animals were fasted overnight and administered with a single oral dose (20 mg/kg) of WNTinib as prepared above. Blood samples were collected at pre-determined time points [0 (pre-dosing), 0.5, 1, 2, 4, 8, 12, and 24 hours] through the retro-orbital plexus (using micro hematocrit capillary tubes) into microvette CB300 K2E tubes. A sparse sampling technique (four mice per time point) was adopted during blood collection so that blood loss from each mouse was kept less than 10% of the total blood volume. Plasma was harvested by centrifuging the blood using a Sorvall Legend Micro 21R centrifuge (Thermo Scientific) at 3000 rpm for 15 minutes and stored at −80±100 until further analysis. Mice were allowed to access feed 2 hours post-dosing and water ad libitum. Pharmacokinetic analysis of these compounds was performed using a Shimadzu UFLC prominence system (equipped with degasser (DGU-20A5), isopump (LC-20AD), autosampler (SIL-20AC HT) and column oven (CTO-20AC)) connected to a Sciex 4000 Q-Trap LC/MS/MS with heated electrospray ionization source. The primary stock solution (1.0 mg/mL) of WNTinib and Carbamazepine (Internal Standard, IS) were prepared in DMSO and used for subsequent dilutions. Required secondary and working stocks were prepared from the primary stocks using acetonitrile:water (75:25, v/v) as a diluent for the calibration curve (CC) and quality control (QC) studies. For the pharmacokinetic analysis, the mouse plasma was mixed with carbamazepine (50 ng/ml, final concentration) and extraction solvent (70% acetonitrile and 0.1% formic acid) and shaken for 10 minutes. After vigorous shaking, the samples were centrifuged at 14000 rpm for 20 minutes and the supernatant was collected. Chromatographic resolution of compound and the IS was achieved by injecting 1-2 μL of the processed sample on a Luna C18 100 Å column (50×2.0 mm, 5 μm; Phenomenex, USA) maintained at room temperature using a mobile phase consisting of 5 mM ammonium formate (buffered with 0.1% formic acid) and acetonitrile delivered at a flow-rate of 0.4 mL/minute. Quantitation was carried out using the multiple reaction monitoring of the transitions.

### Statistics

Statistics were done in Prism 9 (GraphPad). Comparisons between groups were calculated with either two-tailed paired or unpaired t tests (distinct testing). For comparisons between multiple groups, ANOVA tests were used (F and degrees of freedom noted, repeated measure). For survival analyses, log rank P values were calculated (repeated measure).

### Example 8—WNTinib Selectively Antagonizes CTNNB1-Mutated HCC

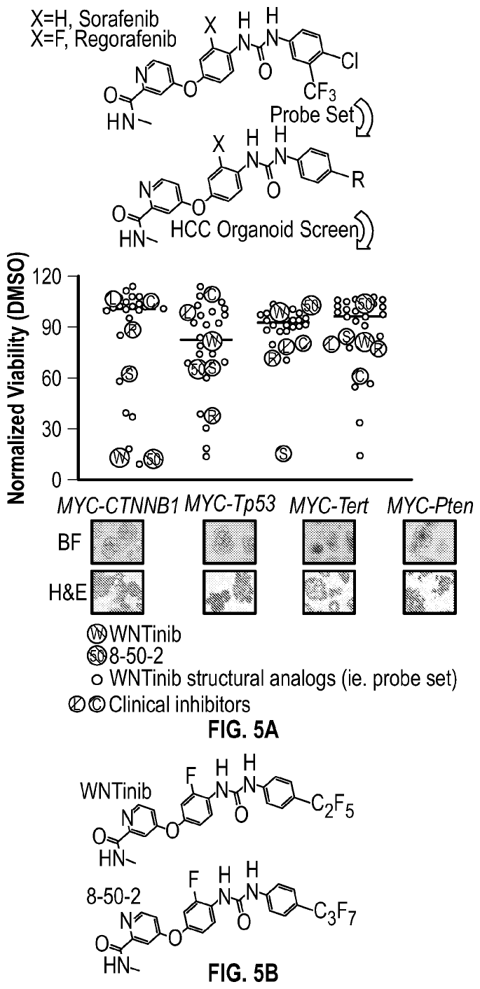

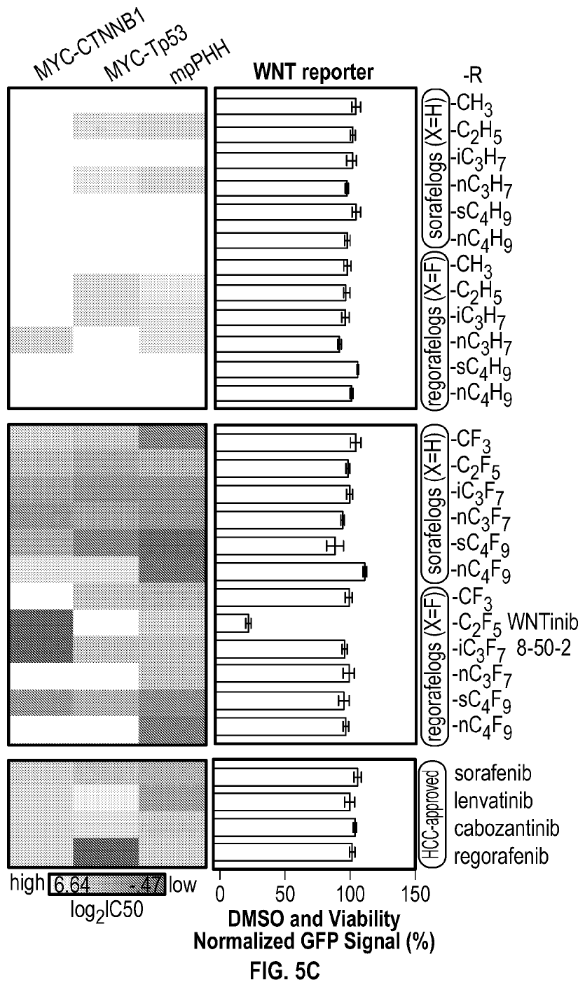

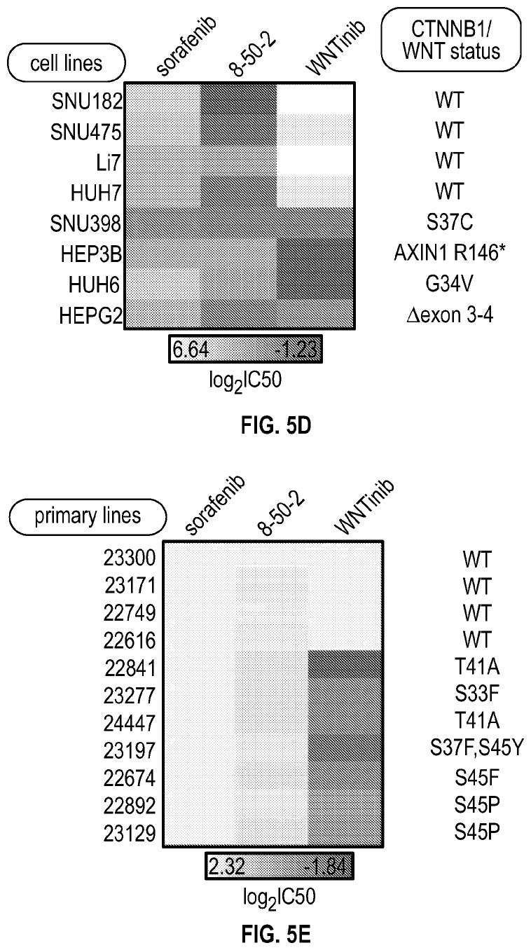

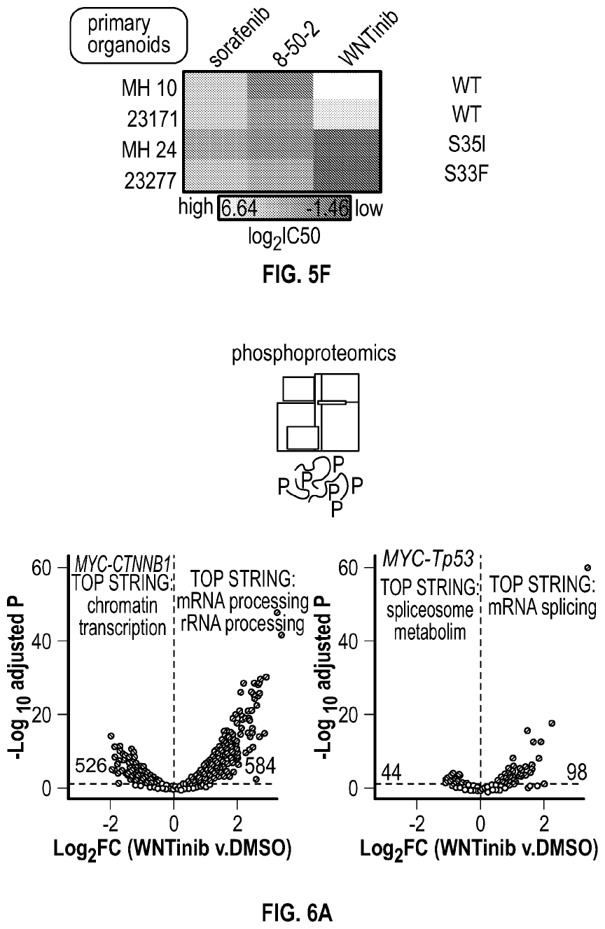

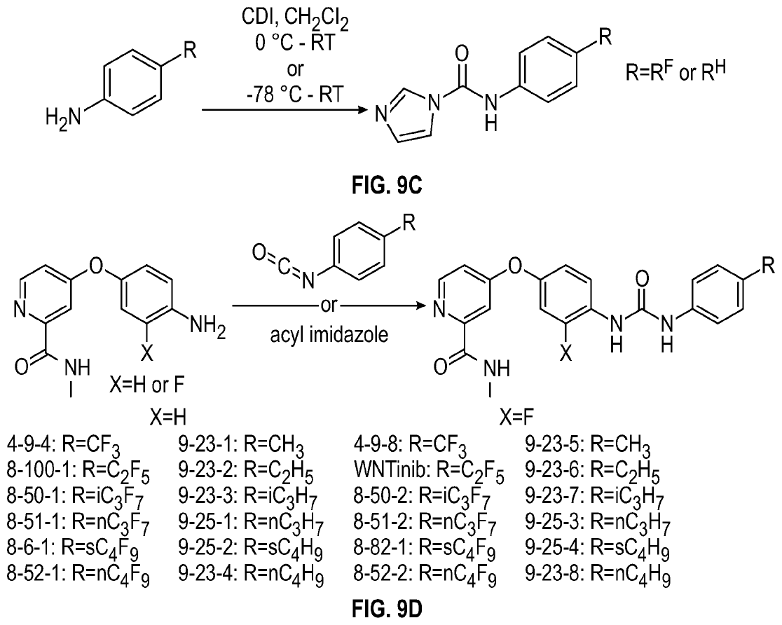

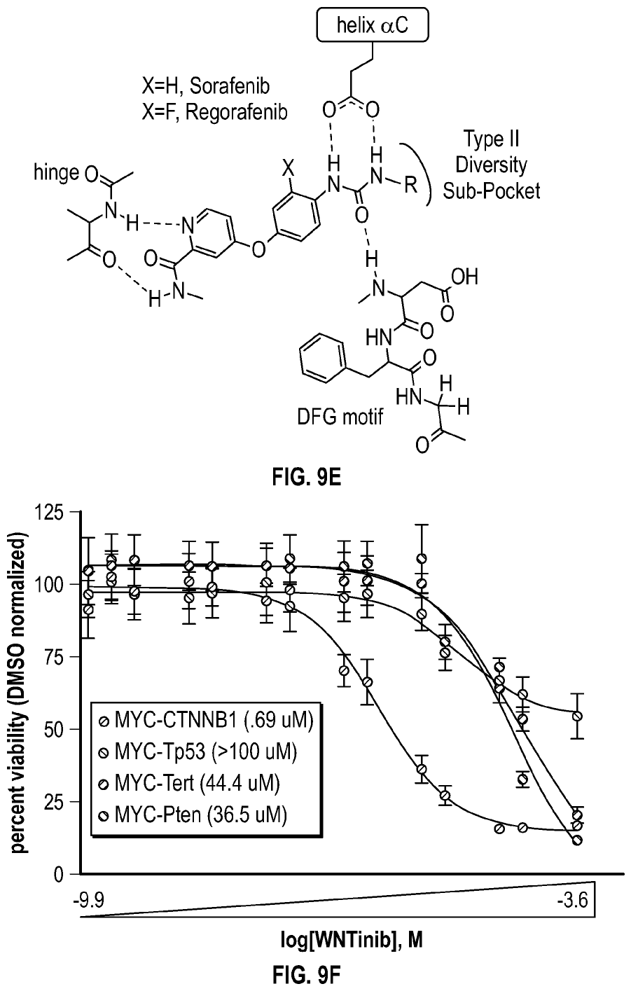

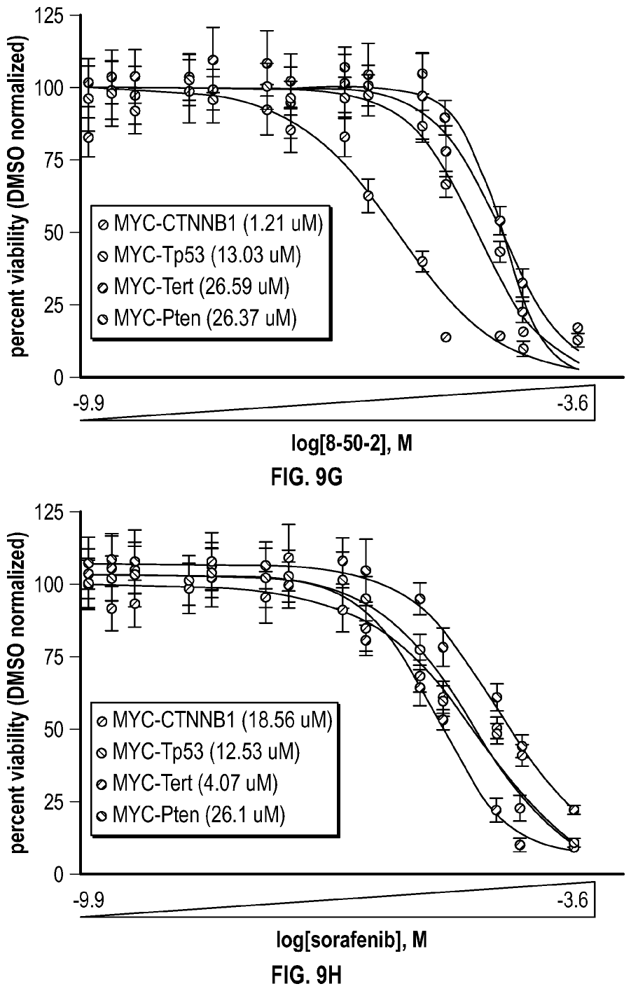

Multi-targeted kinase inhibitors (multi-KIs) have demonstrated clinical success in HCC; however, these drugs display limitations including relatively non-selective target-inhibition profiles. Applicant recently developed a chemical strategy for the diversification and disease-specific improvement of tool and FDA-approved multi-KIs (Dar et al., “Chemical Genetic Discovery of Targets and Anti-Targets for Cancer Polypharmacology,” Nature 486(7401):80-84 (2012); Sonoshita et al., “A Whole-Animal Platform to Advance a Clinical Kinase Inhibitor into a New Disease Space,” Nat. Chem. Biol., 14(3):291-298 (2018); and Yu et al., “Phenotype-Based Screens with Conformation-Specific Inhibitors Reveal a p38 Gamma and Delta as Targets for HCC Polypharmacology,” Mol. Cancer Ther. 18(9):1506-1519 (2019), which are hereby incorporated by reference in their entirety). Using regorafenib and sorafenib as starting molecules, a library of small molecules that are differentially substituted with hydrocarbon and perfluoroalkane groups at a type II diversity sub-pocket that is predicted to distinguish kinase targets was developed (FIG. 5A, FIGS. 9A-9E).

These small molecules were screened in four tumor organoid models derived from genetically-engineered mouse models of HCC (FIG. 5B) (Molina-Sanchez et al., “Cooperation Between Distinct Cancer Driver Genes Underlies Intertumor Heterogeneity in Hepatocellular Carcinoma,” Gastroenterology 159(6): 2203-2220 (2020), which is hereby incorporated by reference in its entirety). The tumor organoids shared overexpression of MYC and possessed unique oncogenic second hits: (I) overexpression of Δ90 β-catenin (MYC-CTNNB1), (II) deletion of p53 (MYC-Tp53), (III) overexpression of Tert (MYC-Tert) and (IV) deletion of Pten (MYC-Pten). This screen yielded potent and selective activity for WNTinib) in the MYC-CTNNB1 tumor organoids. A close analog (8-50-2) was additionally identified and will be used as a comparison for further downstream analysis. (FIG. 5B, FIGS. 9F-9I).

The library was further refined by performing dose-response curves on tumor organoids and primary human hepatocytes (FIG. 1C). In addition, stable MYC-CTNNB1 organoids expressing a GFP-based WNT reporter were created to assess direct transcriptional inhibition of the pathway. Substitution of length and branch-varying hydrocarbon chains conferred little activity, though activity markedly improved with certain perfluoroalkane substitutions. WNTinib was exemplary for its therapeutic index, as defined by its preferential activity in the CTNNB1-driven model as compared to hepatocytes. WNTinib was additionally the only compound to downregulate WNT-driven GFP expression. Notably, clinical kinase inhibitors did not display a similar therapeutic index, nor did they repress the WNT pathway reporter (FIG. 1C).

WNTinib's unique specificity was further confirmed by using a comprehensive set of WNT-driven HCC models (i.e., of CTNNB1- or AXIN1-mutants): publicly available human HCC cell lines (FIG. 5D), primary patient-derived HCC cell lines (FIG. 5E), and primary patient-derived HCC organoids (FIG. 5F).

### Example 9—WNTinib Represses Wnt Pathway Transcriptional Output

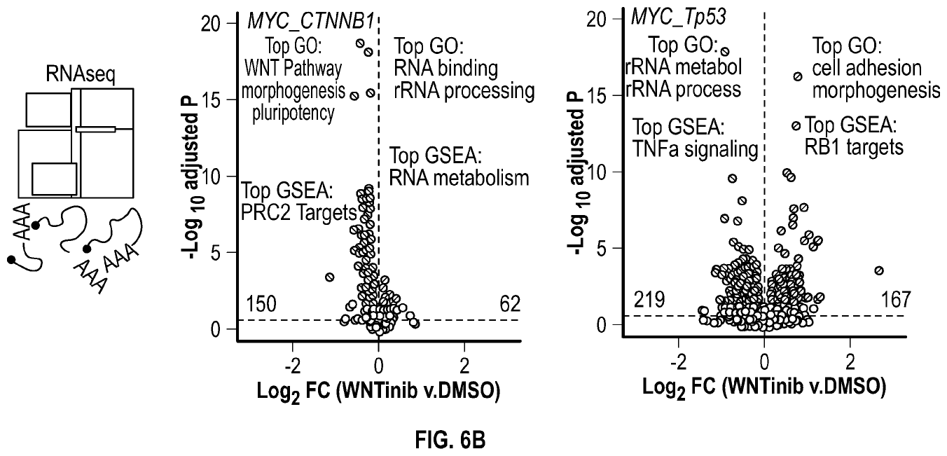

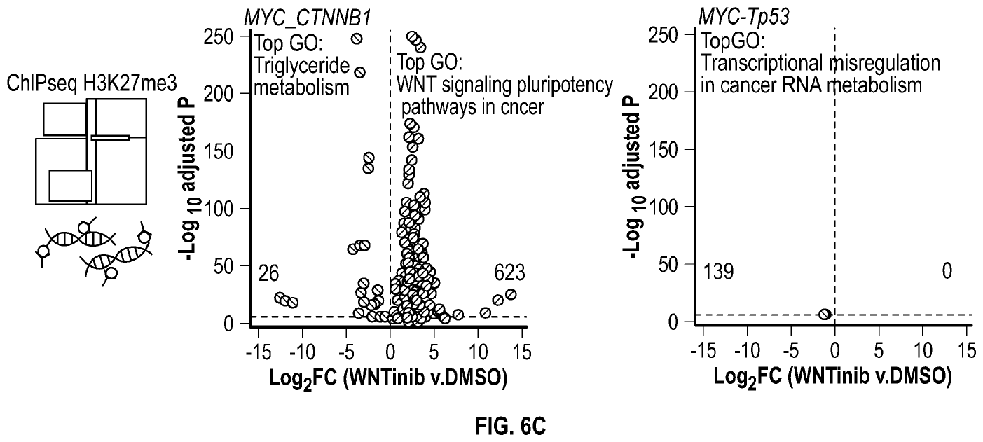

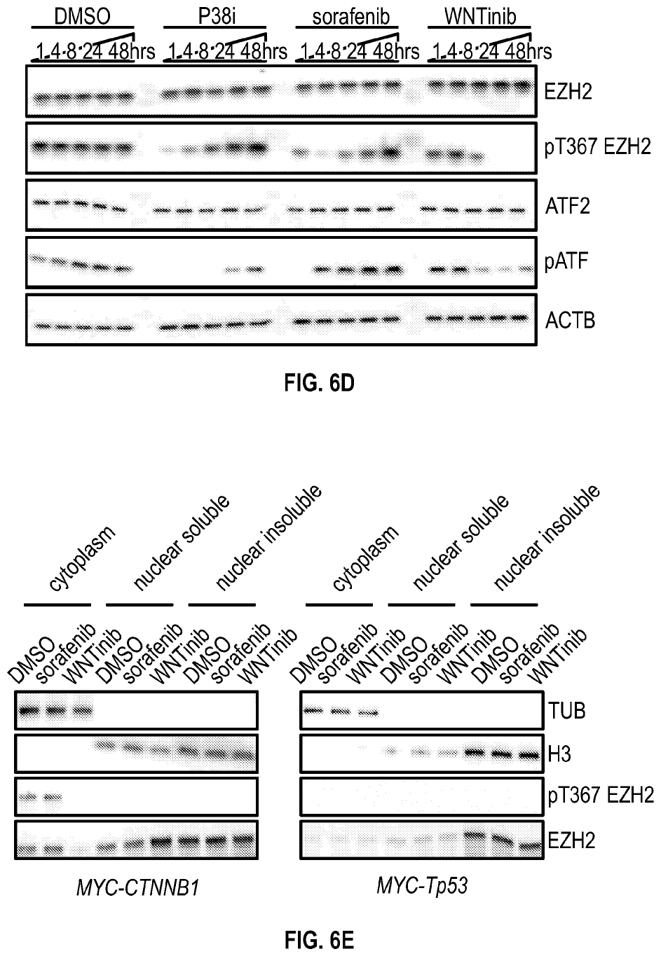

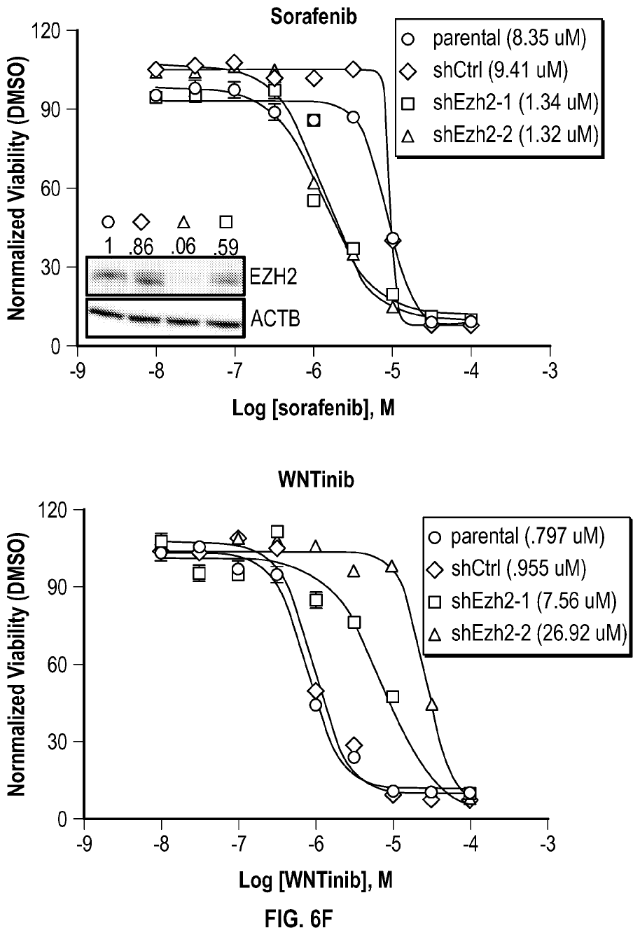

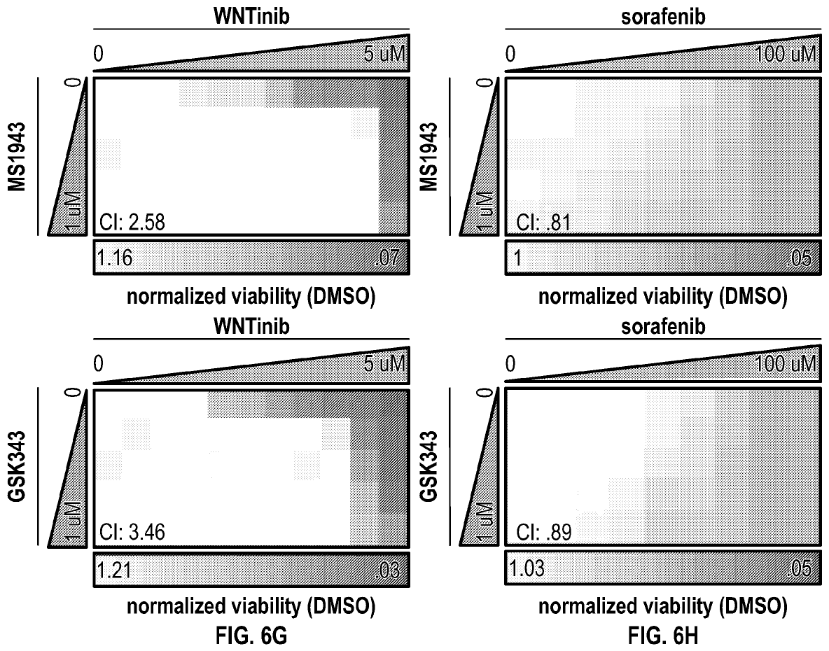

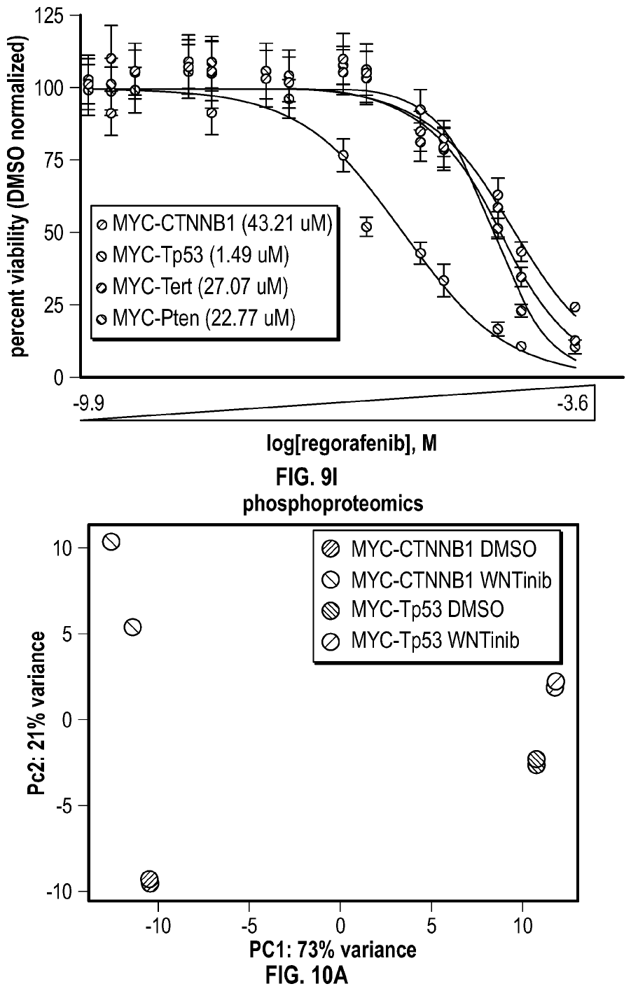

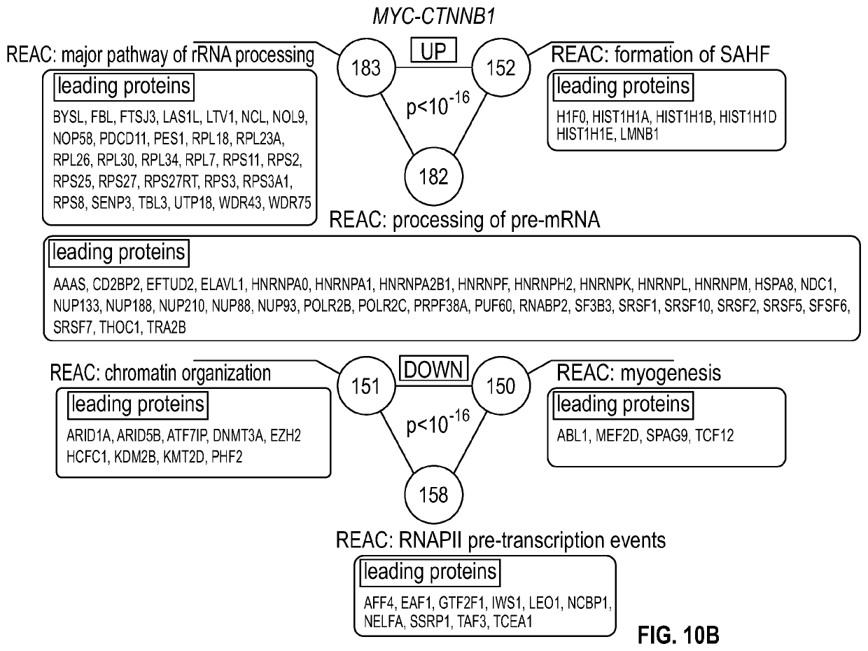

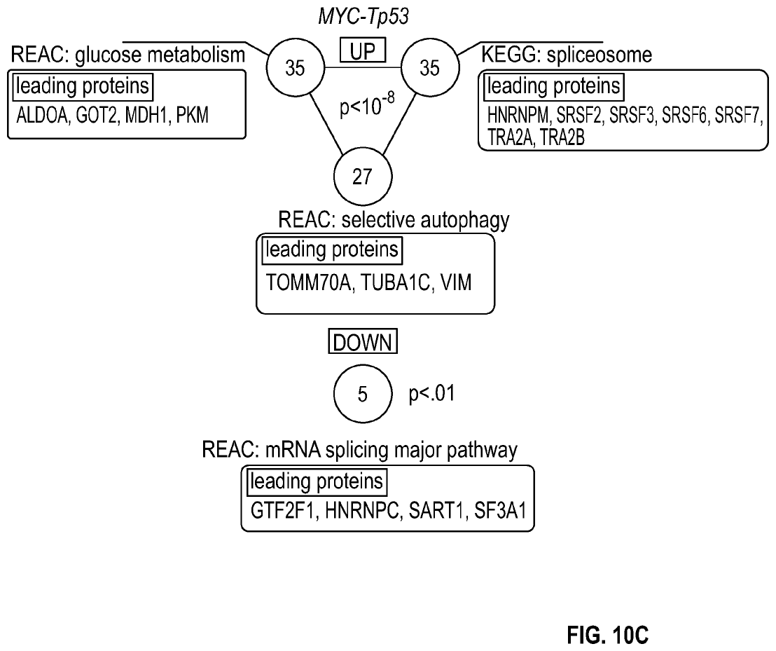

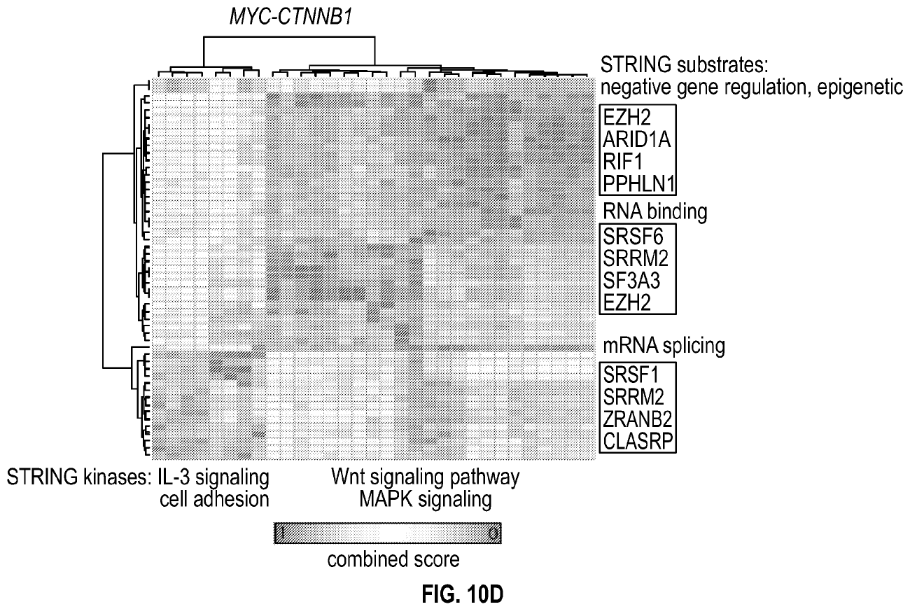

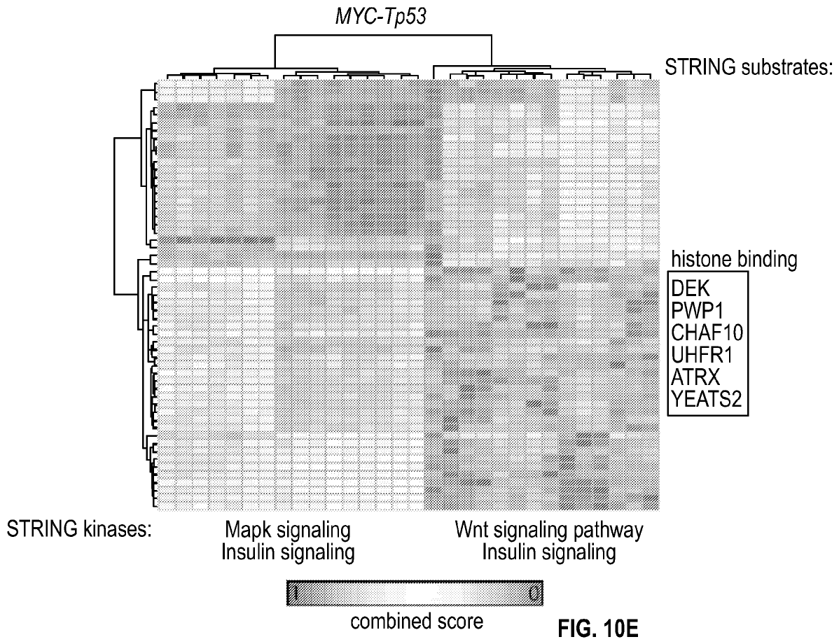

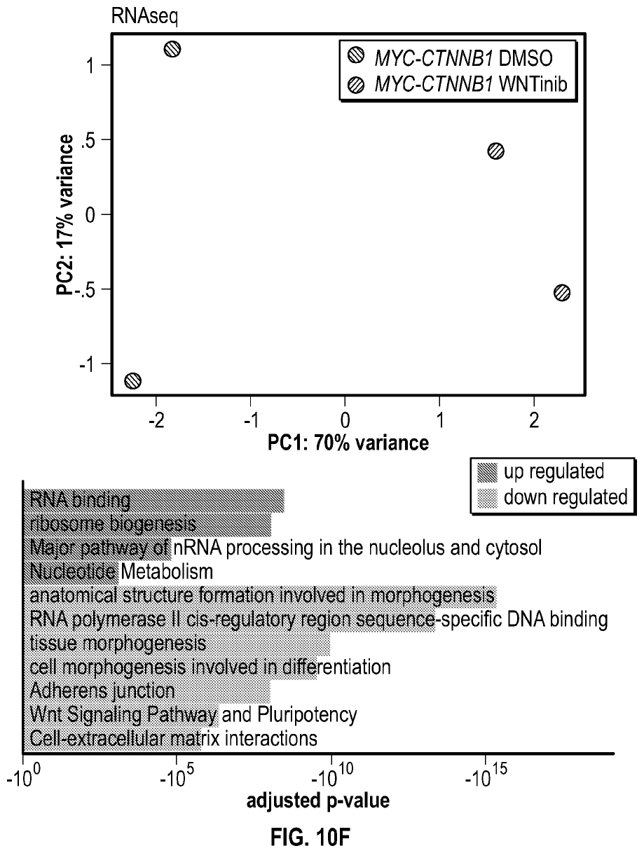

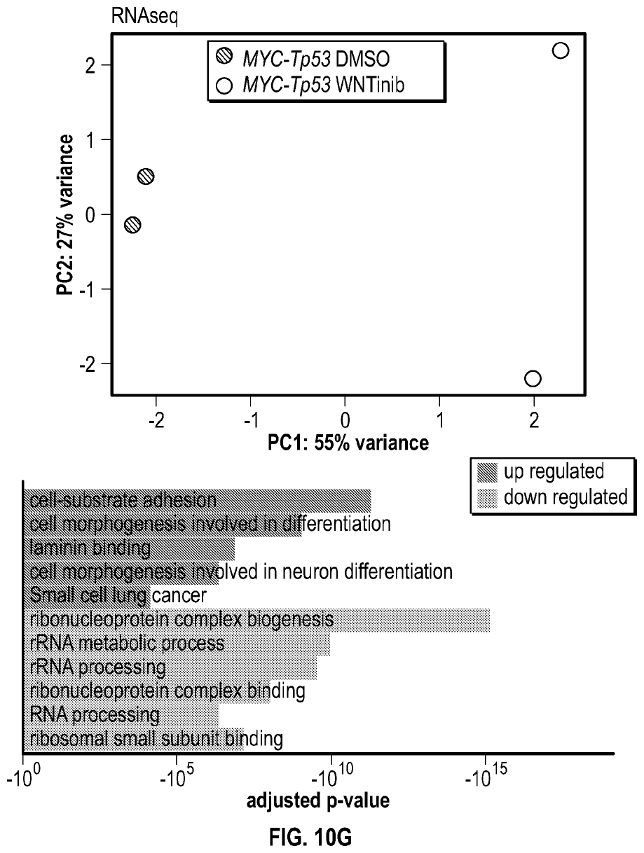

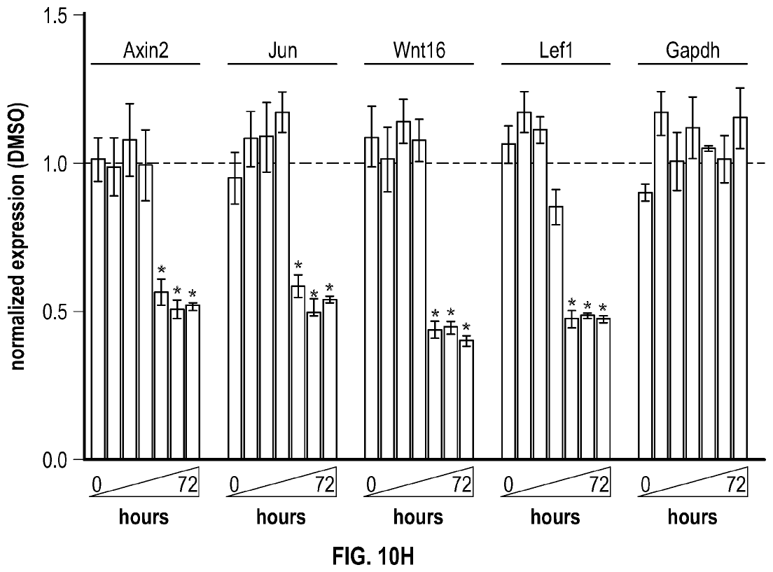

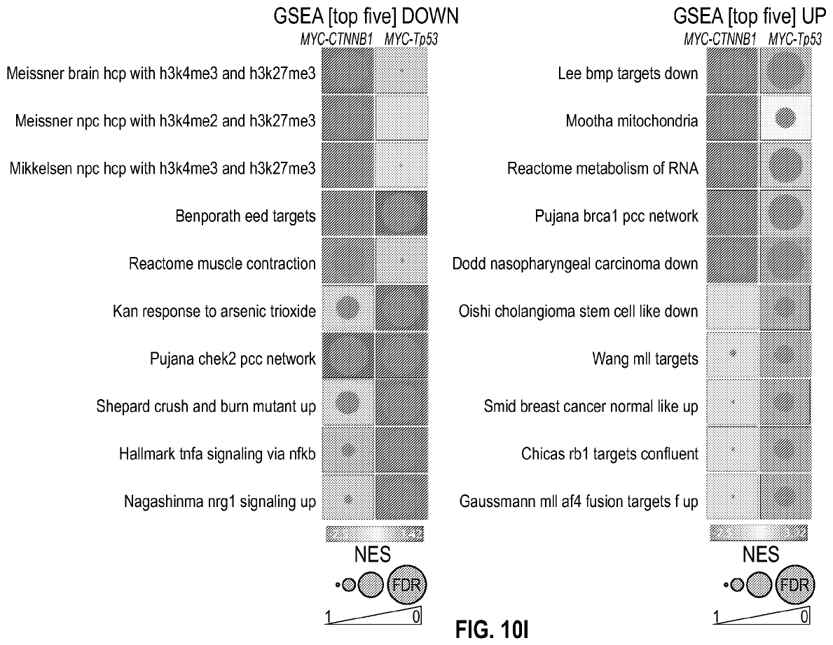

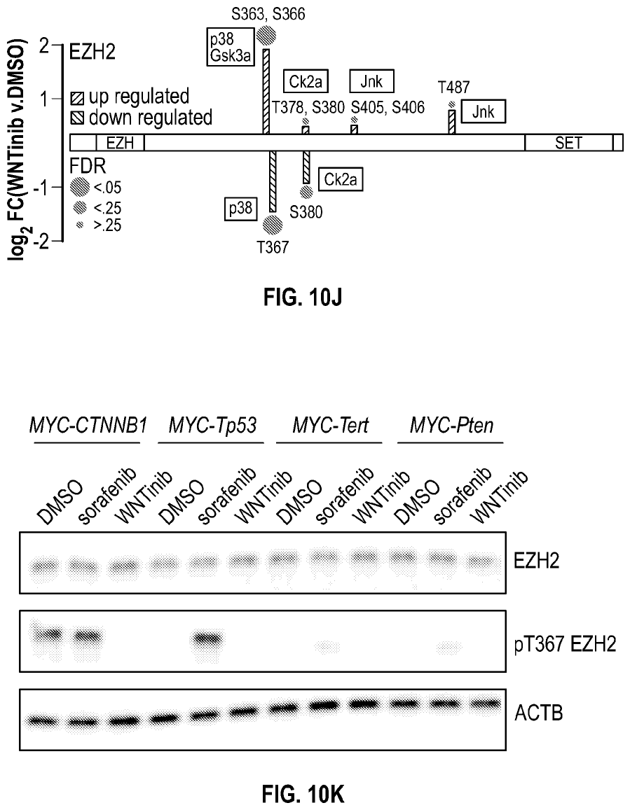

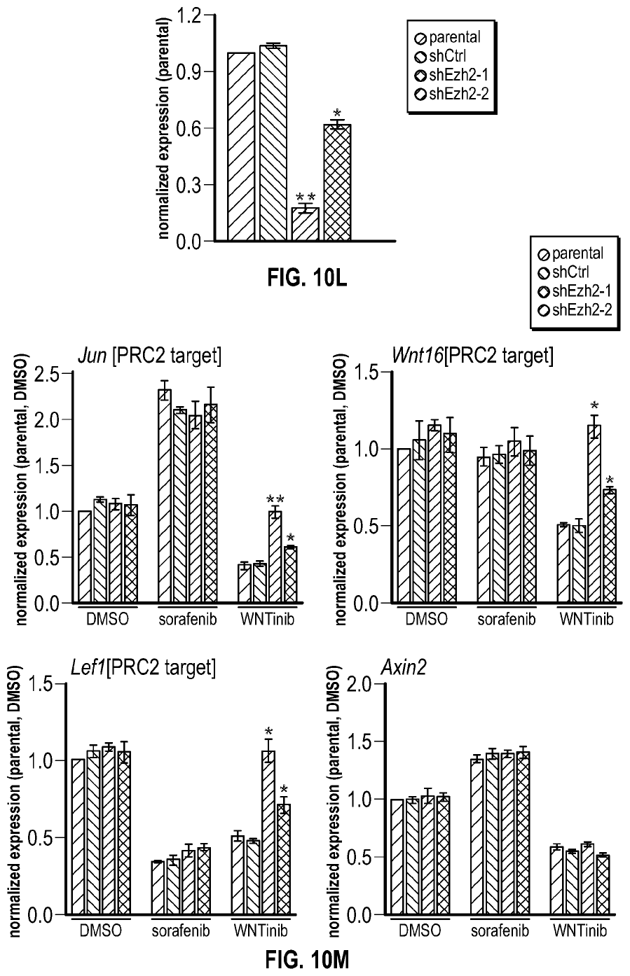

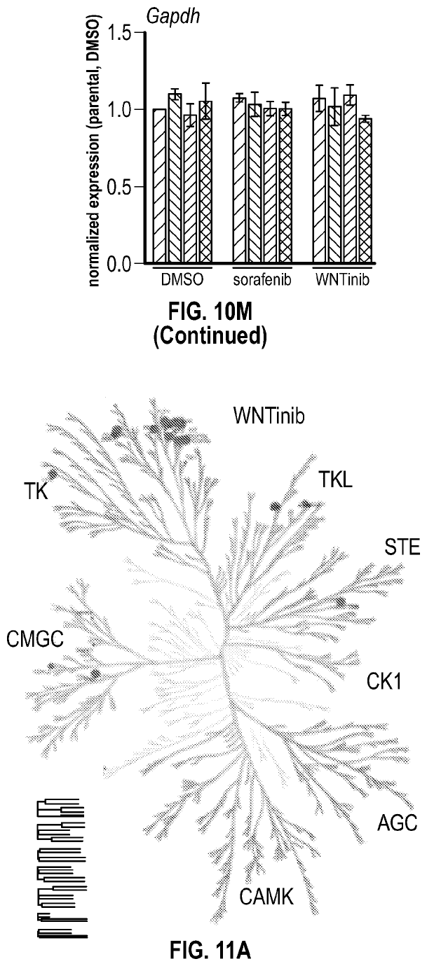

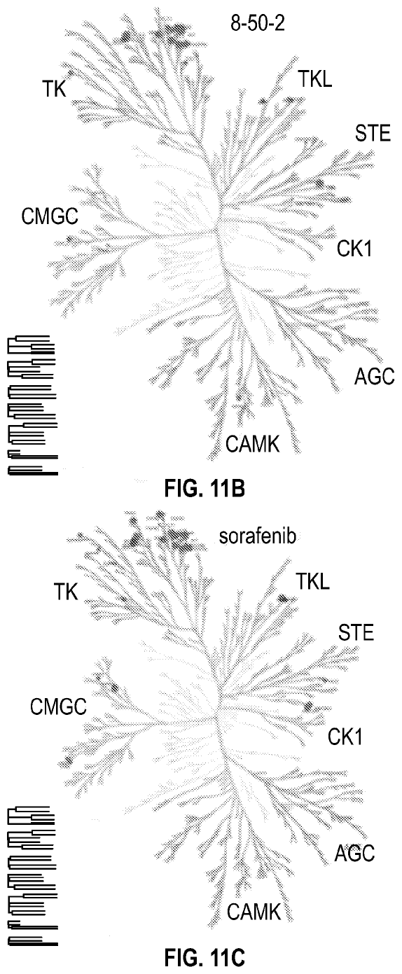

In order to uncover WNTinib's mechanism of action, unbiased multiomic experiments were performed in tumor organoids. First, a phosphor-proteomic analysis was performed in WNTinib sensitive (MYC-CTNNB1) and resistant (MYC-Tp53) tumor organoids. After 24 hours of treatment, WNTinib modulated a higher number of phosphoproteins in the CTNNB1-driven model (FIG. 6A). Network enrichment (STRING) for differentially upregulated phosphorylation events identified proteins in the RNA metabolism and splicing pathways, which were redundant in both organoid models. Downregulated phosphorylation events in the MYC-CTNNB1 organoids were instead unique and enriched for proteins involved in transcription and chromatin organization (FIGS. 6A, FIGS. 10A-10C).

Next, an analysis to assess global signaling event perturbations by WNTinib was performed, which yielded two main results: (I) kinase-substrate mapping depicted inhibition landscapes elicited by WNTinib in MYC-CTNNB1 and MYC-Tp53 organoids, which converged on kinases belonging to Wnt and MAPK signaling pathways, and (II) these pathways were regulated in the MYC-CTNNB1 organoids to uniquely change the phosphorylation of substrates belonging to proteins involved in the negative epigenetic regulation gene expression (FIGS. 10D-10E). These results further linked WNTinib's potential mechanism to chromatin biology. As such, RNA sequencing was conducted in MYC-CTNNB1 and MYC-Tp53 organoids treated with WNTinib for 24 hours to detect downstream transcriptional changes. Differential gene expression analyses revealed two meaningful results: (I) WNTinib-mediated transcriptional regulation was divergent between the two models, and (II) downregulated genes in the MYC-CTNNB1 model were enriched for Wnt signaling, morphogenesis, and pluripotency (FIG. 6B, FIGS. 10F-10H).

Gene set enrichment analysis (GSEA) revealed a strong and specific correlation between downregulated genes in the MYC-CTNNB1 organoids and transcriptional bivalency (genes with H3K4me3 and H3K27me3) regulated by the polycomb repressive complex 2 (PRC2) (FIG. 10I). Consistent with this result, WNTinib treatment in MYC-CTNNB1 organoids upregulated the tri-methylation of lysine 27 on histone H3 (H3K27me3) at the promoters of genes belonging to Wnt and pluripotency pathways as assessed by ChIP-sequencing. Importantly, this was not seen in the MYC-Tp53 model organoids (FIG. 10C).

### Example 10—EZH2 is Essential for the Activity of WNTinib

Phosphorylation of threonine 367 on EZH2, a known p38 target (Anwar et al., “p38-Mediated Phosphorylation at T367 Induces EZH2 Cytoplasmic Localization to Promote Breast Cancer Metastasis,” Nat. Commun. 9:2801 (2018) and Consalvi et al., “Praja1 E3 Ubiquitin Ligase Promotes Skeletal Myogenesis through Degradation of EZH2 upon p38 Activation,” Nat. Commun. 8:13956 (2017), which are hereby incorporated by reference in their entirety), was the most significantly downregulated phosphorylation event spanning the EZH2 protein (FIG. 10K). This site is also known to control the cellular localization of EZH2, whereby loss of phosphorylation licenses EZH2 into the nucleus for gene repression (Anwar et al., “p38-Mediated Phosphorylation at T367 Induces EZH2 Cytoplasmic Localization to Promote Breast Cancer Metastasis,” Nat. Commun. 9:2801 (2018), which is hereby incorporated by reference in its entirety).

WNTinib's temporal regulatory impact on p38 targets pT367 EZH2 and pT71 ATF2 was assessed in comparison to sorafenib and the p38 inhibitor SB202190 (p38i). In contrast to sorafenib and p38i, which displayed transient inhibition of pT367 EZH2 and pT71 ATF2, presumably due to compensatory feedback signaling, WNTinib elicited delayed, but durable inhibition up to 48 hours on these same markers (FIG. 6D). The levels of pT367 EZH2 and its modulation by either WNTinib or sorafenib was also assessed in the four tumor organoid models that were originally screened. Notably, pT367 was only detectable at baseline and also inhibited by WNTinib in the MYC-CTNNB1 model (FIG. 10K). Additionally, sorafenib induced pT367 across several organoid models (FIG. 10K), suggesting a general feedback mechanism elicited by this compound as related to the modulation of EZH2 phosphorylation. These results, taken together, imply a unique biological dependency linking pT367 EZH2 to the fitness of CTNNB1-mutated HCC that can be specifically antagonized by WNTinib.

Next, EZH2 localization in WNTinib sensitive MYC-CTNNB1 and insensitive MYC-Tp53 tumor organoids was investigated. EZH2 localized diffusively in the MYC-CTNNB1 model but was primarily nuclear insoluble in the MYC-Tp53 model (FIG. 6E; lanes 7-9, left and right panels). Upon WNTinib treatment in the MYC-CTNNB1 organoids, pT367 EZH2 in the cytoplasm was ablated (FIG. 6E; compare lane 3 to 1, pT367 blot), which coincided with an increase in nuclear partitioning of EZH2 (FIG. 6E; compare lanes 6 to 4, total EZH2). In comparison, sorafenib did not impact phosphorylation or nuclear to cytoplasmic ratios of EZH2 (FIG. 6E, compare lanes 2 to 1 and 5 to 1). These experiments demonstrate that WNTinib-induced increases in nuclear EZH2, which correlates with increased H3K27me3 deposition and transcriptional repression (FIGS. 6B-6C), distinguish WNTinib from sorafenib, and further suggested that EZH2 may be essential for WNTinib's mechanism of action.

This model was tested using two orthogonal methods: (I) shRNA depletion of EZH2, and (II) pharmacological inactivation of EZH2. MYC-CTNNB1 organoids were transduced with two independent shRNAs targeting EZH2 and the response to WNTinib was measured. WNTinib activity was markedly reduced and proportional with depletion efficiency (FIG. 6F, FIG. 10I). MS1943 (a small molecule EZH2 degrader) or GSK343 (a selective EZH2 inhibitor) were used in combination with WNTinib to observe dose-escalation impacts on WNTinib activity (Verma et al., “Identification of Potent, Selective, Cell-Active Inhibitors of the Histone Lysine Methyltransferase EZH2,” ACSMed. Chem. Lett. 3(12):1091-1096 (2012) and Ma et al., “Discovery of a First-in-Class EZH2 Selective Degrader,” Nat. Chem. Biol. 16:214-222 (2020), which are hereby incorporated by reference in their entirety). Consistent with a necessary function in the activity of WNTinib, degradation or inhibition of EZH2 via MS1943 and GSK343, respectively, antagonized the activity of WNTinib but did not alter the dose response to sorafenib in MYC-CTNNB1 organoids (FIGS. 6G-6H).

Further, three putative PRC2 targets (Jun, Wnt16, and Lef1) and two non-targets (Axin2, Gapdh) were evaluated to examine cooperation between WNTinib and EZH2 at the transcriptional level. MYC-CTNNB1 organoids depleted for EZH2 and treated with WNTinib were able to restore expression of PRC2 targets, which did not occur with non-targets or with sorafenib treatment (FIG. 6M). Together, this data demonstrates that WNTinib requires EZH2 for the selection inhibition of Wnt targets in β-catenin mutant HCC.

### Example 11—WNTinib Utilizes Unique Polypharmacology to Regulate the EZH2-WNT Axis

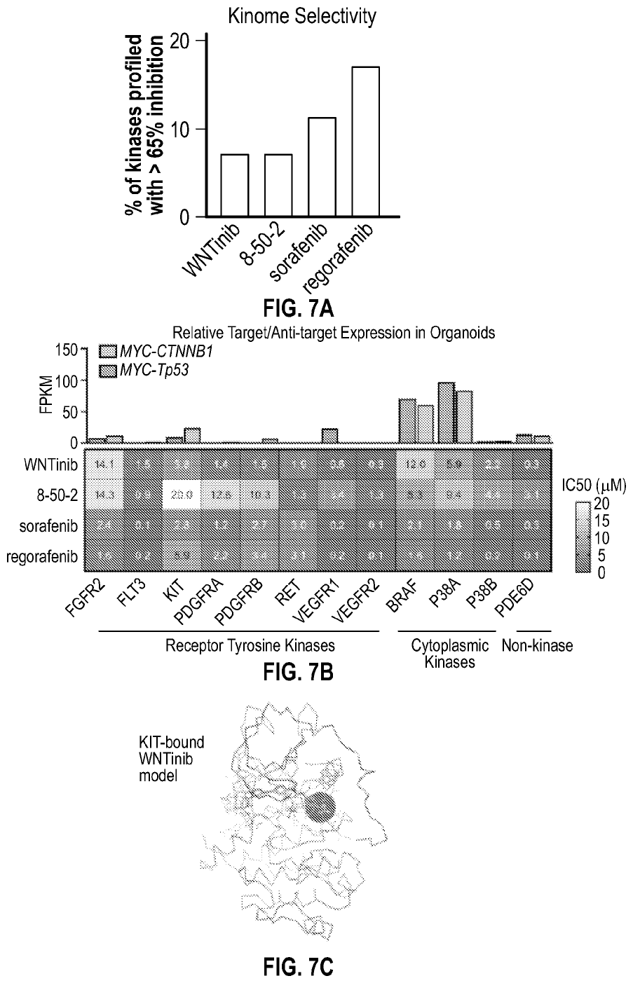

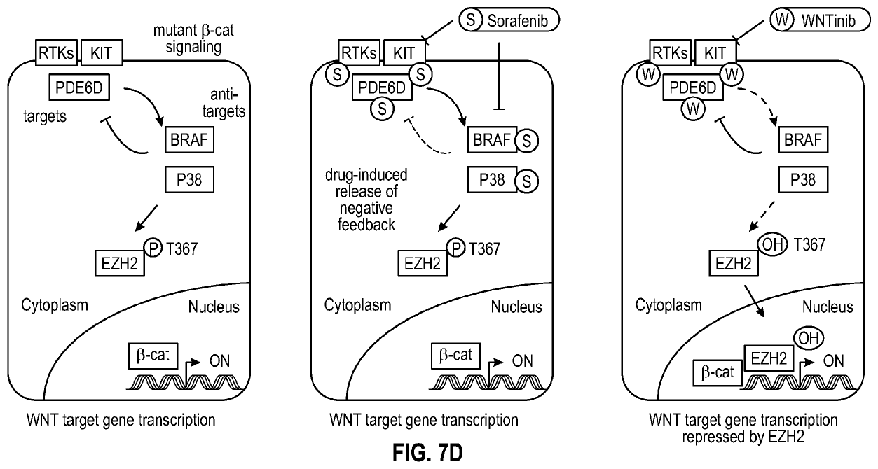

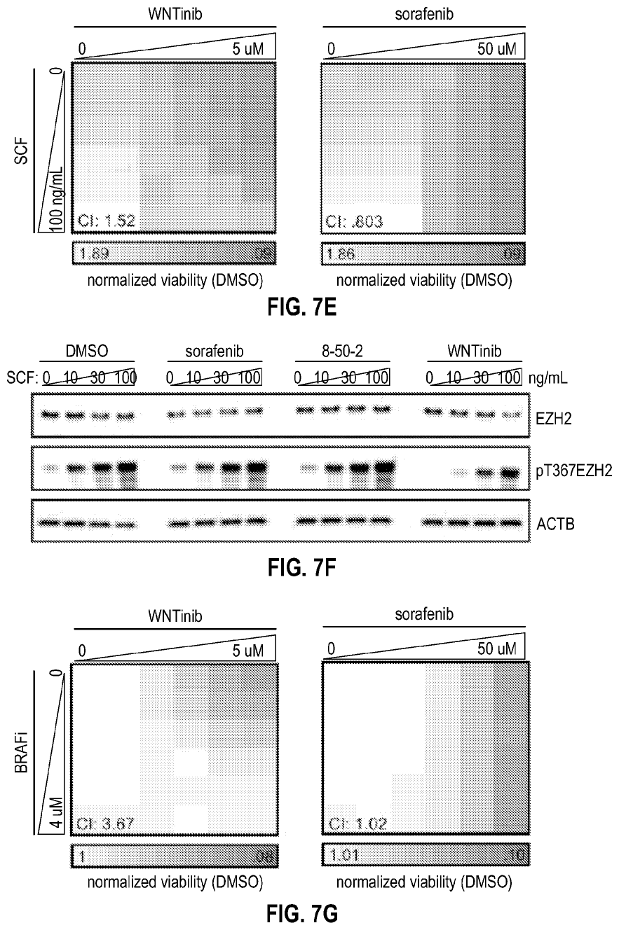

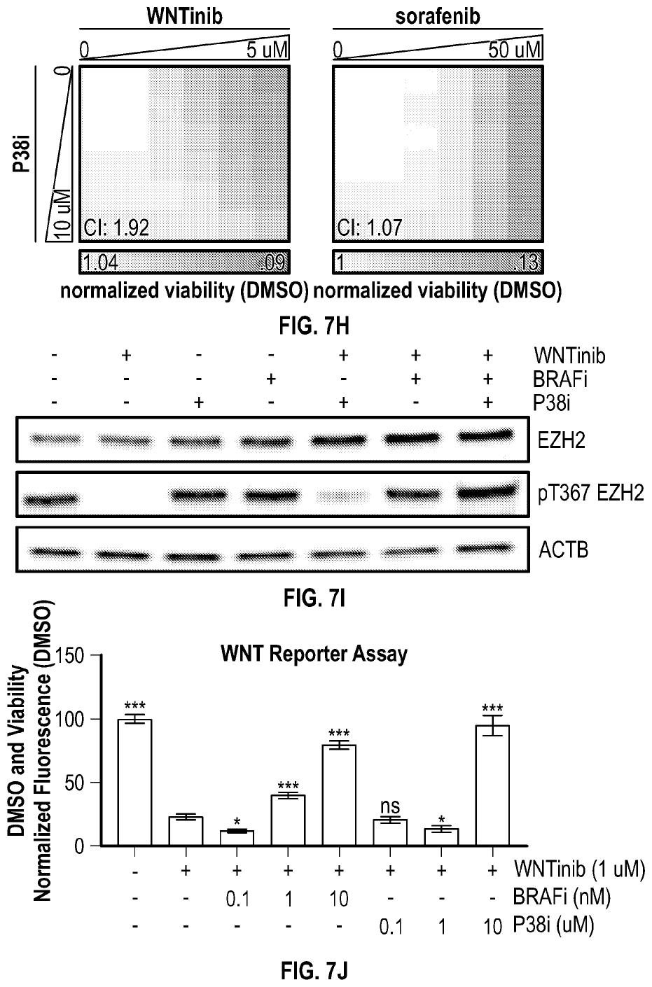

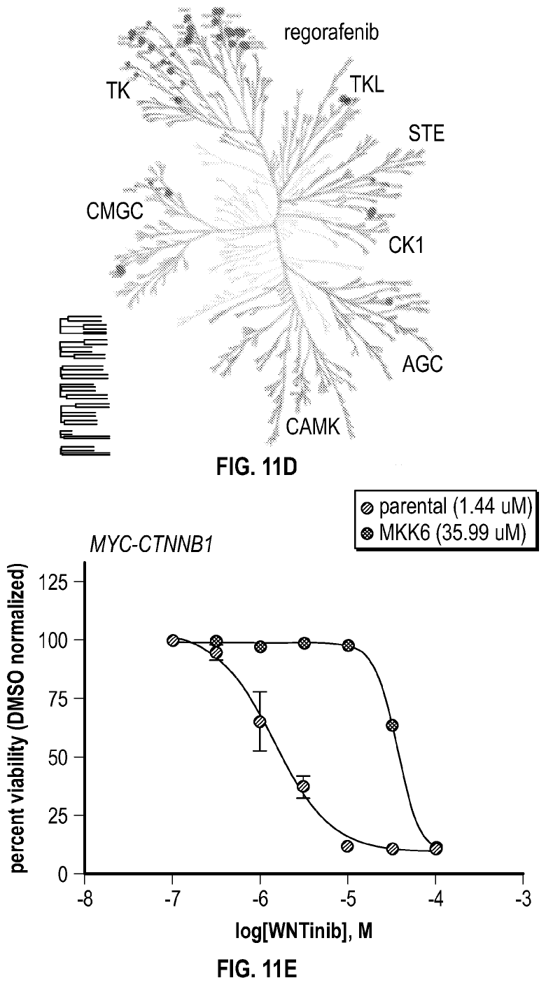

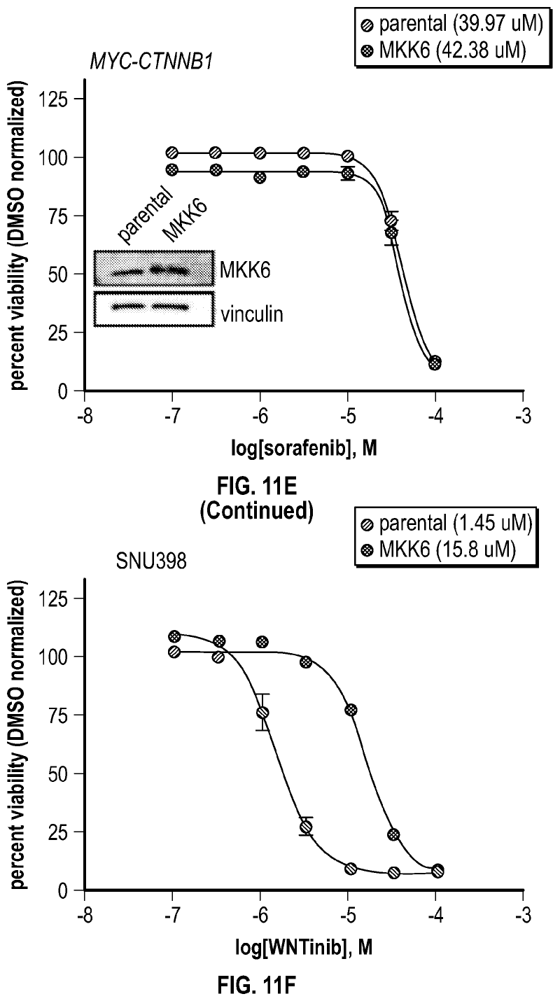

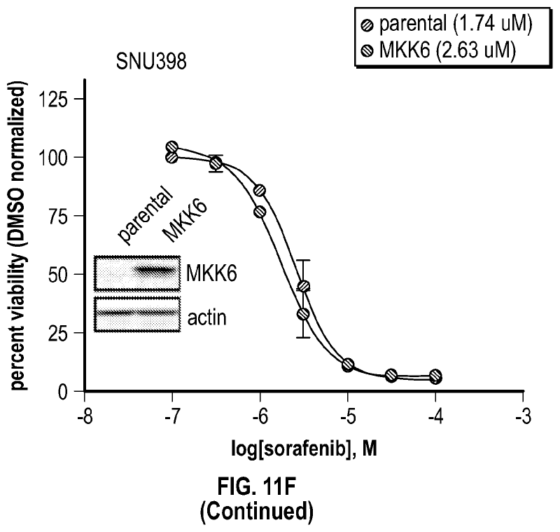

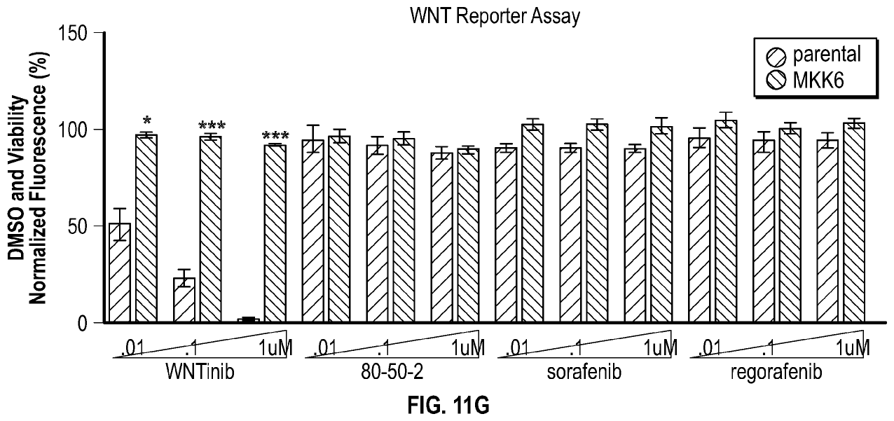

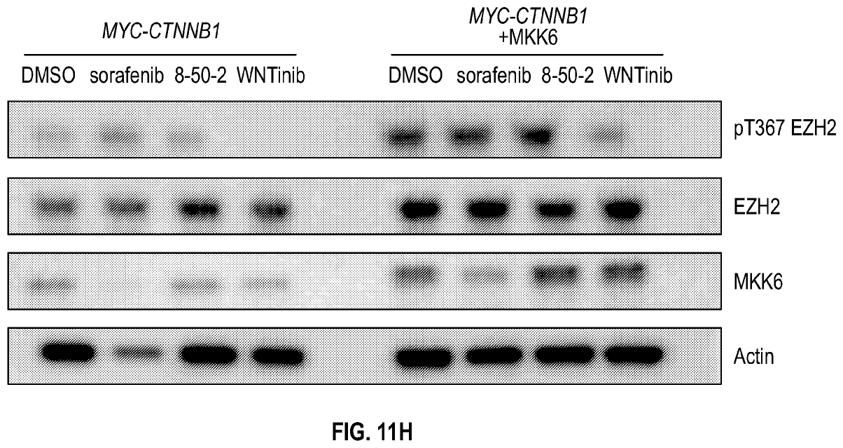

In order to characterize the direct targets of WNTinib, kinome selectivity profiles were first assessed by in vitro kinase assays. WNTinib maintains several of the proposed key targets of sorafenib and regorafenib, including c-KIT, PDGFRα/β, VEGFR1/2, RET and FLT3 (FIG. 7A, FIGS. 11A-11D); however, WNTinib is generally less promiscuous than the parental compounds based on selectivity scores (Davis et al., “Comprehensive Analysis of Kinase Inhibitor Selectivity,” Nat. Biotechnol. 29(11):1046-51 (2011), which is hereby incorporated by reference in its entirety). Small molecule inhibition profiles can differ between in vitro assays and those completed in a cellular context (Robers et al., “Quantifying Target Occupancy of Small Molecules within Living Cells,” Annu. Rev. Biochem. 89:557-581 (2020), which is hereby incorporated by reference in its entirety). Protein kinases and other classes of enzymes or targets often reside within large macromolecular assemblies, which can alter drug binding kinetics or target engagement. To verify WNTinib activity on critical targets directly in vivo, a modified version of bioluminescence resonance energy transfer was used (NanoBRET) (Robers et al., “Quantifying Target Occupancy of Small Molecules within Living Cells,” Annu. Rev. Biochem. 89:557-581 (2020), which is hereby incorporated by reference in its entirety). Quantification of the NanoBRET signal provides in vivo drug target engagement and kinetic information (i.e., IC₅₀ and residence time) for WNTinib on targets of interest. Relative to sorafenib and regorafenib, WNTinib retained a similar in vivo IC₅₀ on several receptor tyrosine kinases thought to be critical to the mechanism of sorafenib and regorafenib, including KIT and VEGFR1/2 (FIG. 7B, bottom panel). WNTinib was docked on the DFG-out structure of KIT, and consistent with the experimental data, a compatible binding pose with the —C₂F₅ perfluoroalkane buried in the type II inhibitor pocket was found (FIG. 7C, red circle). Further, through in vivo target engagement assays, it was found that WNTinib was markedly less potent than sorafenib and regorafenib on several cytoplasmic kinases including BRAF and p38α/β (FIG. 7B, bottom panel). The latter are potentially important in WNT-driven HCCs, given their high expression in the model (FIG. 713, top panel). Moreover, comparison of WNTinib with the close structural analog 8-50-2 further revealed notable structure-activity relationships (SAR) with significant differences in binding on KIT and its downstream effector the prenyl-binding protein PDE6D. This comparative SAR provided the hypothesis that was next evaluated—that WNTinib's unique ability to block WNT-driven tumors derives from co-inhibition of the KIT/MAPK pathway in the absence of concurrent and direct engagement on BRAF and p38α/β kinases (FIG. 7D).

To test modulation of KIT/MAPK/EZH2 signaling by WNTinib, MYC-CTNNB1 tumor organoids were first stimulated with the KIT ligand stem cell factor (SCF) to activate the receptor. Consistent with the model, only WNTinib and not sorafenib activity was antagonized (Combination Index CI>1) by titration of SCF (FIG. 7E). Mechanistically, addition of SCF promoted EZH2 phosphorylation at T367, and partially rescued WNTinib-mediated repression of this phosphorylation event (FIG. 7F).

Next, the hypothesis that inhibition of both BRAF and p38a/P could elicit negative feedback loops, thereby specifically diminishing the efficacy of WNTinib, was evaluated. Titration of both a BRAFi (dabrafenib) (FIG. 7G) and a p38i (SB202190) (FIG. 7H) strongly antagonized WNTinib activity (CI>1), while this was not the case for sorafenib, supporting the notion that the reduced inhibition of BRAF and p38α/β (FIG. 7B) contributes to the unique activity of WNTinib. Mechanistically, combining BRAF and p38α/β inhibitors with WNTinib restored EZH2 phosphorylation at T367 (FIG. 7I), and furthermore rescued WNTinib-mediated repression of a WNT reporter (FIG. 7J). Together, this data strongly supports the designation of BRAF and p38α/β as disease specific anti-targets in β-catenin mutant HCC.

As additional evidence for the relevance of the inhibition of the KIT/MAPK/EZH2 pathway for WNTinib's mode of action, CTNNB1-mutant organoids and cells (SNU398) were engineered to stably express constitutively-active MKK6 (S207E, T211E), which is an upstream activator of p38 family kinases and thereby pT367 EZH2. Consistent with a specific upstream role of WNTinib on the KIT, MKK6-overexpressing CTNNB1 mutant models were markedly rescued from the activity of WNTinib but not sorafenib (FIGS. 11E-11F). Additionally, MKK6 overexpression rescued WNTinib-mediated repression of a WNT reporter and EZH2 T367 phosphorylation inhibition (FIGS. 11G-11H).

Taken together, this data demonstrates a specific vulnerability in CTNNB1-mutated HCC that is dependent on nuclear EZH2 repressive activity on WNT targets (FIGS. 6A-6H) mediated by the unique action of WNTinib on RTKs including KIT, with release of negative feedback signaling avoided relative to sorafenib due to reduced engagement of BRAF and p38α/β anti-gargets (FIG. 7D).

### Example 12—WNTinib Outperforms HCC-Approved Therapeutics In Vivo

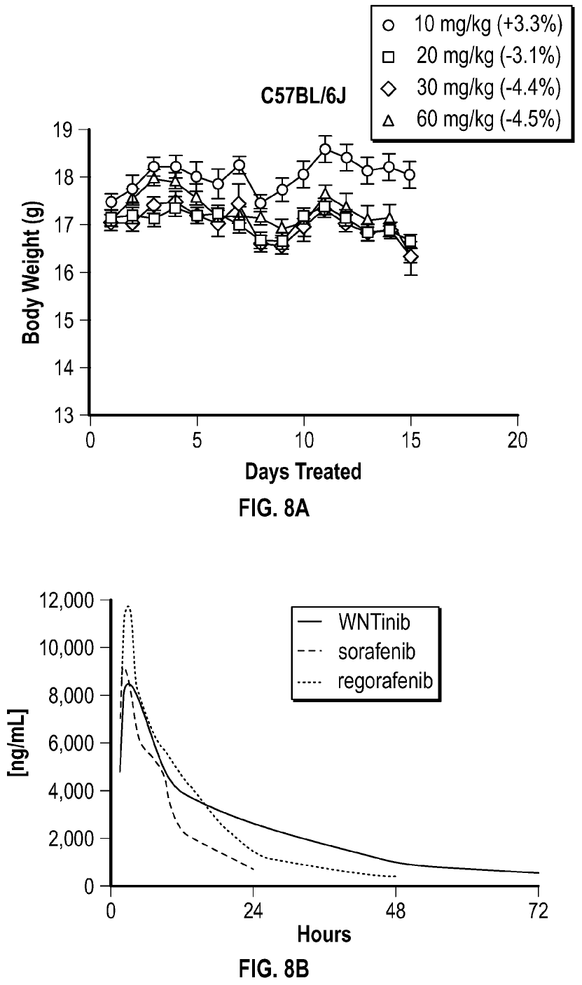

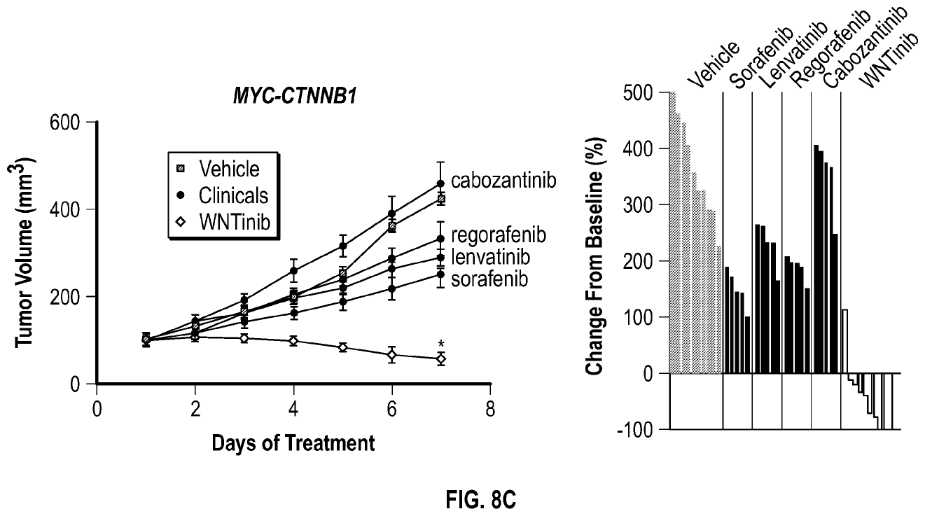

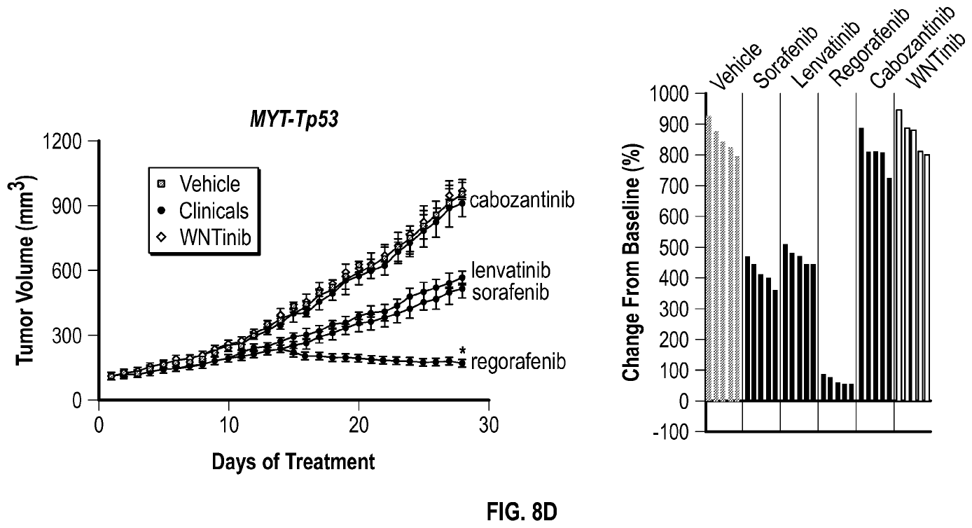

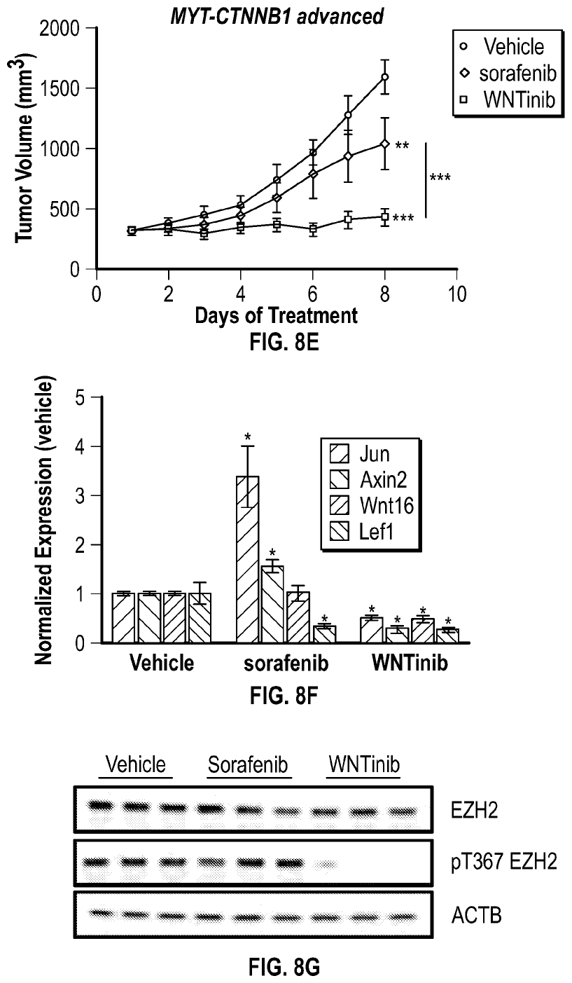

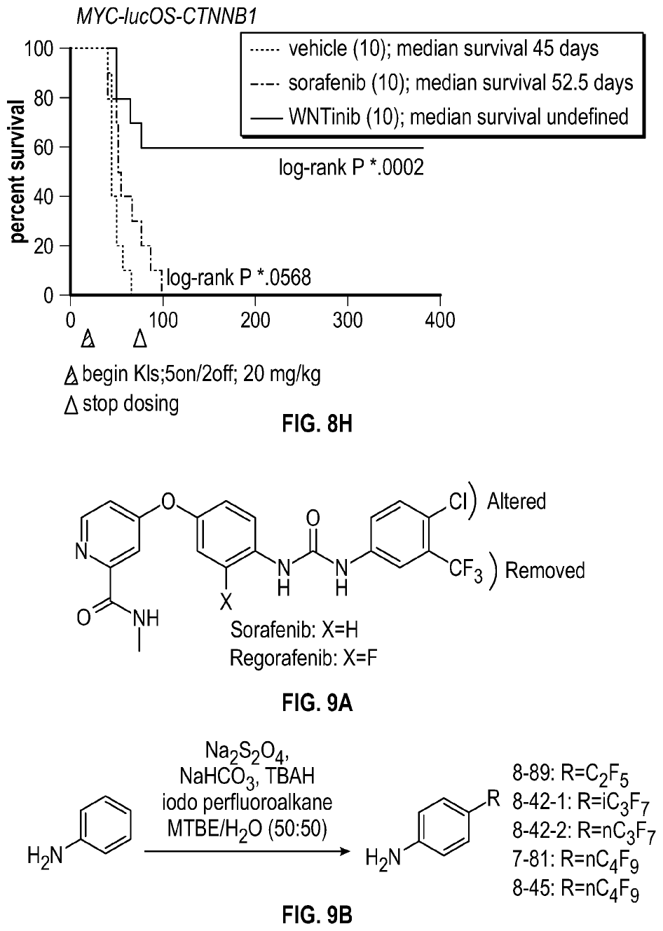

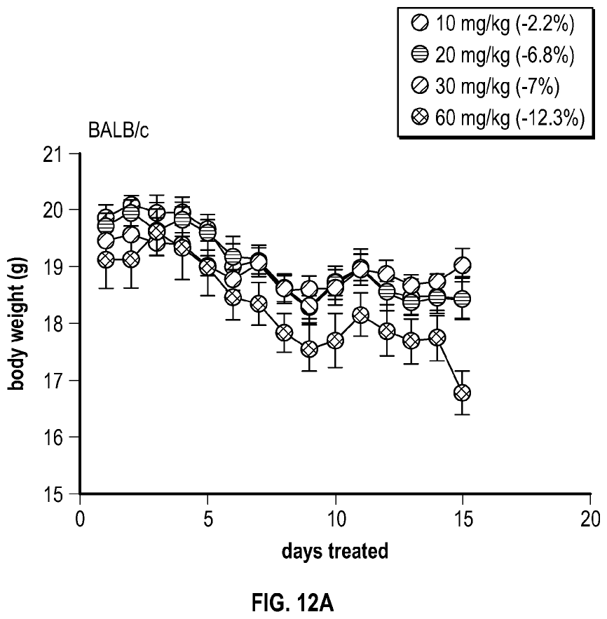

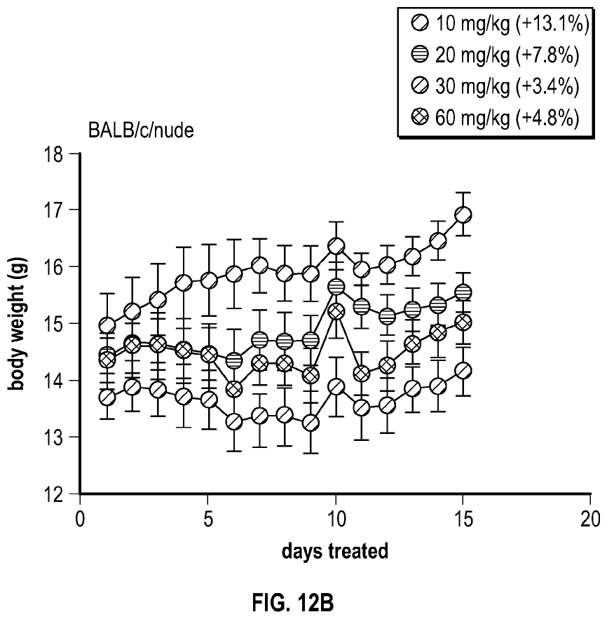

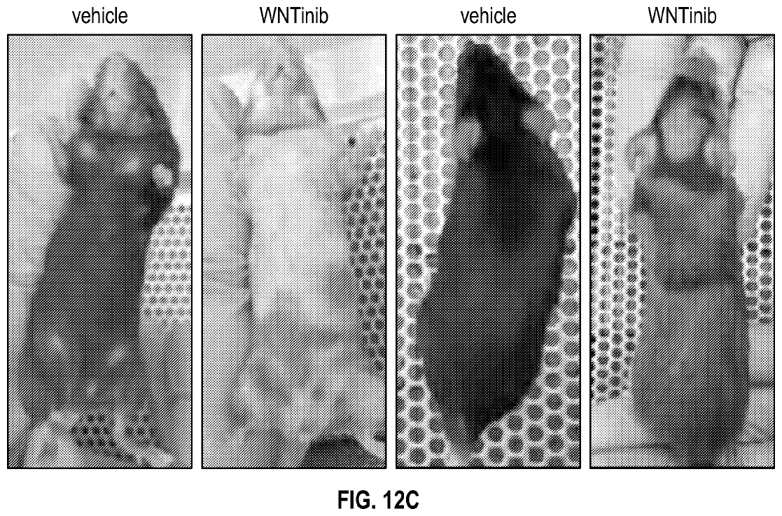

To rationalize a treatment regimen for WNTinib and understand potential toxicity, in vivo dose escalation was performed in three mouse strains (FIG. 8A, FIGS. 12A-12B). Mice were dosed daily for two weeks, and body weight loss was used as a proxy for systemic toxicity. No mice showed signs of sickness, though weight loss did occur in a dose-dependent manner.

To further tailor the dosing of WNTinib, in vivo pharmacokinetics were performed at a dose of 20 mg/kg. This approach revealed several physiochemical distinctions of WNTinib relative to sorafenib, including a long half-life (9.38 hours vs 8 hours), a concentration of 7 μM 24 hours after oral dosing, and detectable quantities of drug in blood plasm up to 72 hours post dosing (FIG. 8B). Based on this data, preclinical insight into appropriate dosing of WNTinib was achieved (i.e., daily oral gavage at 20-30 mg/kg).

MYC-CTNNB1 tumor organoids were engrafted into C57BL/6 mice and treatment was started when tumor volume reached 100 mm³. The four FDA-approved kinase inhibitors displayed a spectrum of activity, but were not able to induce significant tumor regression (FIG. 8C). WNTinib instead induced significant tumor regression in the majority of animals, thereby validating the in vitro results (FIG. 8C). MYC-Tp53 tumor organoids were also engrafted, and in this model, consistent with the in vitro data, WNTinib was inactive while lenvatinib, sorafenib, and regorafenib had better activity (FIG. 8D). These results confirm the in vitro specificity observations described herein and indicate that WNTinib operates in a vulnerable chemical space for CTNNB1-mutated HCC. As a further confirmation for the sustained on-target KIT inhibition, it was observed that mice treated for >3 weeks with WNTinib displayed a unique phenotype of fur depigmentation (FIG. 12C) (Moss et al., “Hair Depigmentation is a Biological Readout for Pharmacological Inhibition of KIT in Mice and Humans,” J. Pharmacol. Exp. Ther. 2:476-480 (2003), which is hereby incorporated by reference in its entirety). Next, the experiment was repeated, allowing MYC-CTNNiBI organoids to engraft and grow to 300-400 mm³ before starting treatment, to better mimic an advanced-stage tumor setting. Once again, WNTinib was superior to the standard of care clinical compound (sorafenib) (FIG. 8E). Mechanistically, this correlated with in vivo suppression of WNT-target genes (FIG. 8F) and inhibition of EZH2 T367 phosphorylation (FIG. 8G).

An autochthonous CTNNB1-driven HCC model, which has been used as a model for resistance to immunotherapy and closely recapitulates therapeutic efficacy observed in patients (Ritz de Galarreta et al., “β-Catenin Activation Promotes Immune Escape and Resistance to Anti-PD-1 Therapy in Hepatocellular Carcinoma,” Cancer Discov. 9:1124-1141 (2019), which is hereby incorporated by reference in its entirety), was next used to test WNTinib efficacy. Mice were treated with either sorafenib or WNTinib, and animals were observed for survival. Sorafenib provided a modest survival benefit of 7.5 days. WNTinib conferred a significant survival benefit, where only 40% of its treatment arm succumbed to death with the remaining animals alive over 300 days post final dosing (FIG. 12H). Overall, these in vivo efficacy studies highlight that the unique mechanism and target profile of WNTinib strongly distinguishes this compound from multiple clinical KIs in vivo. This data further supports the development of WNTinib as the first precision medicine for β-catenin mutant and Wnt-activated tumors.

### Discussion of Examples 8-12

This work demonstrates a multidisciplinary approach for the discovery and characterization of personalized therapeutics for HCC. The examples presented herein identify WNTinib as a novel multitargeted kinase inhibitor with specific efficacy in CTNNB1-mutated HCC. Phosphoproteomic, RNA-seq, and ChiP-seq data provide insight into this compound's unique mechanism of action, revealing reactivation of EZH2 and the commensurate repression of Wnt and pluripotency genes as a unique vulnerability in the CTNNB1-mutant model. EZH2's contribution to HCC biology has been studied using multiple models, though no study has investigated its function, or specifically nuclear reactivation, in mutationally-defined settings (Bugide et al., “Inhibition of Enhancer of Zeste Homolog 2 (EZH2) induces Natural Killer Cell-Mediated Eradication of Hepatocellular Carcinoma Cells,” Proc. Natl. Acad. Sci. USA 115(5):E3509-E3518 (2018); Gao et al., “EZH2 Represses Target Genes through H3K27-Dependent and H3K27-Independent Mechanisms in Hepatocellular Carcinoma,” Mol. Cancer Res. 12(10):1388-1397 (2014); and Xiao et al., “EZH2 Negatively Regulates PD-L1 Expression in Hepatocellular Carcinoma,” J. Immunother. Cancer 7(1):300 (2019), which are hereby incorporated by reference in their entirety). Kinase inhibitors for HCC, including sorafenib and its related analogs, imparted limited efficacy in both pre-clinical models and patients. The chemical diversification strategy described herein, starting from sorafenib and regorafenib, uncovered WNTinib as a potent and selective antagonist of CTNNB1-mutant HCC, suggesting that non-specific clinical kinase inhibitors may represent ideal starting points for new drug development campaigns. In fact, sorafenib has been shown to dampen Wnt signaling transiently (Lachenmeyer et al., “Wnt-Pathway Activation in two Molecular Classes of Hepatocellular Carcinoma and Experimental Modulation by Sorafenib,” Clin. Cancer Res. 18(18):4997-5007 (2012), which is hereby incorporated by reference in its entirety), which supported the use of this compound to identify durable antagonists of the Wnt pathway. Remarkably, the data presented herein suggests that the key differentiating activities of WNTinib are the removal of the original intended target (BRAF) and the widely recognized off-targets (p38α/p) of sorafenib and regorafenib (Wilhelm et al., “Preclinical Overview of Sorafenib, A Multikinase Inhibitor that Targets both Raf and VEGF and PDGF Receptor Tyrosine Kinase Signaling,” Mol. Cancer. Ther. 7(10):3129-3140 (2008), which is hereby incorporated by reference in its entirety).

The designation of BRAF and p38α/β as anti-targets in the context of mutant β-catenin signaling may have been possibly gleaned from their relatively high expression levels (FIG. 7B); however, future experiments will be required to test this hypothesis. Notably, WNTinib inhibits phosphorylation of p38 substrates, including EZH2, on a much later timescale than direct-acting p38 inhibitors and does not elicit detectable compensatory feedback, which could relate to the highly unique profile of WNTinib—specifically on KIT+RTKs+PDE6D. This profile is unprecedented as the first demonstration of a multi-targeted profile specifically on RTKs and a non-kinase adaptor of RAS-MAPK signaling.

Several Wnt pathway inhibitors have failed in clinical development due to toxicity (Jung et al., “Wnt Signaling in Cancer: Therapeutic Targeting of Wnt Signaling beyond Q-Catenin and the Destruction Complex,” Exp. Mol. Med. 52(2):183-191 (2020), which is hereby incorporated by reference in its entirety); rather than directly acting on β-catenin, WNTinib treatment elicits activation of EZH2 to selectively block transcription of Wnt targets. This data suggests that EZH2 activation could be well-tolerated in at least some therapeutic settings. Moreover, this data suggests that EZH2 inactivation may be generally required to open chromatin for oncogenic transcription factors such as mutant β-catenin; such a model for EZH2 would be invisible to conventional target identification strategies based on reverse genetics and could explain why this mechanism has not been documented previously.

In summary, the results presented herein identify that CTNNB1-mutated HCC, which makes up almost a third of human cases, may be actioned with a personalized therapeutic that inhibits Wnt signaling by harnessing EZH2 repression of transcription. These studies provide the rationale to explore WNTinib in proof-of-concept trials with enriched CTNNB1-mutant advanced HCC patients.

### Example 13—Colorectal Cancer (CRC)

Colorectal cancer (CRC) is the third most common and the second deadliest cancer in the world. While hyperactivation of the WNT pathway is the gatekeeper to tumor initiation, it is becoming clearer that the interpatient heterogeneity in advanced CRCs complicates treatment efficacy.

Applicant has taken advantage of genetically engineered mouse organoids models that harbor the most prevalent mutations acquired sequentially during the disease progression. These isogenic models have been generated from healthy wild type (WT) organoids which recapitulate, in vitro, the normal intestinal architecture.

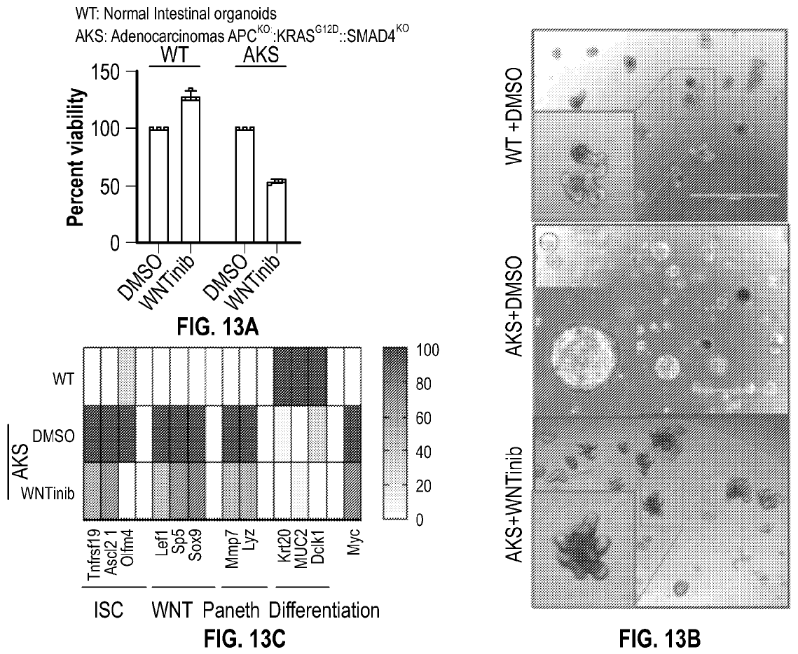

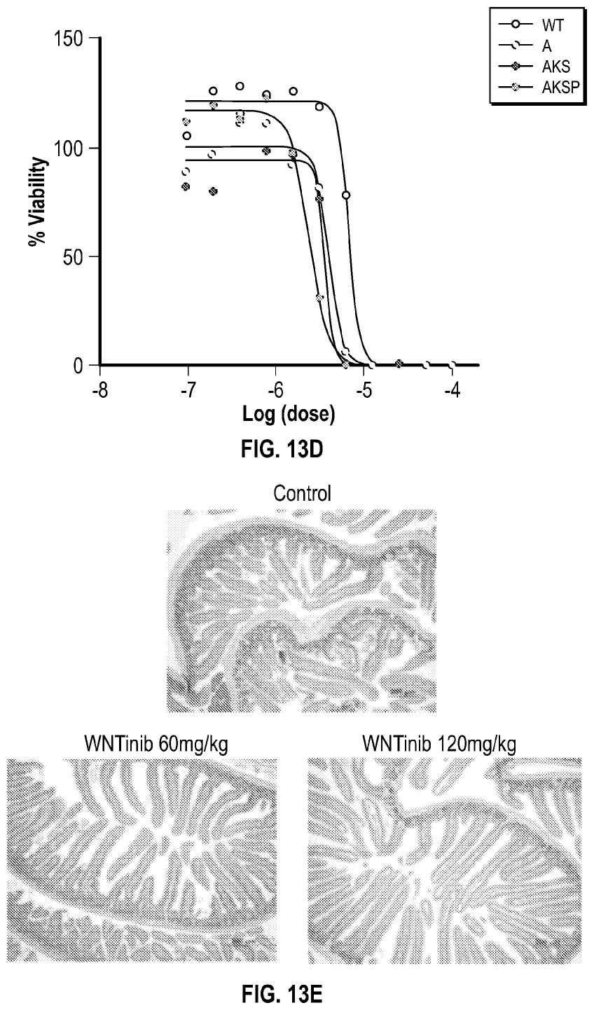

First, an APC deletion (A) was introduced, then a KRAS^{G12D} overexpression (AK) in combination with a SMAD4^{KO} was introduced to affect the TGFbeta pathway (AKS). Last, P53 was deleted to mimic the latest stages of metastatic disease progression (AKSP). WNTinib, is able to reduce the viability of a CRC line (AKS: APC^{KO}::KRAS^{G12D}::SMAD4^{KO}) without affecting that of WT organoids (FIG. 13A). Interestingly, WNTinib is able to induce a reversal of organoid morphology in the AKS organoids: from spherical (typical of transformed tumoroids), to a more structured normal epithelium (typical of WT organoids) (FIG. 13B).

From a molecular standpoint, this reversal in morphology is also backed up by a decrease expression of intestinal stem cell markers (i.e., Tnfrsf19, Ascl2 and Olfm4), WNT-target genes (i.e., Lef1, Sp5 and Sox9), Paneth cell markers (i.e., Mmp7 and Lyz) (FIG. 13C). However, a strong induction of lineage specific markers (i.e., Krt20, Muc2 and Dclk1) was not observed, suggesting that additional drug combinations could be necessary to induce a full reversal of the phenotype.

Next, a full dose-response of WNTinib in WildType normal organoids (WT), APC^{KO} (A), APC^{KO} KRAS^{G12D}/SMAD4^{KO} (AKS) and APC^{KO} KRAS^{G12D}/SMAD4^{KO} P53^{KO} (AKSP) tumoroids was performed. WNTinib has a lower IC50 in the aggressive metastatic AKSP tumoroids compared to their matched parental WT organoid line, providing thus a therapeutic window for the most advanced CRC models (FIG. 13D). To corroborate this observation, no toxicity was observed in mouse intestine 14 days after daily WNTinib treatment with up to 4-fold the therapeutic dose of WNTinib (FIG. 13E).

### Example 14—Combination of WNTinib with Immune Checkpoint Inhibitors

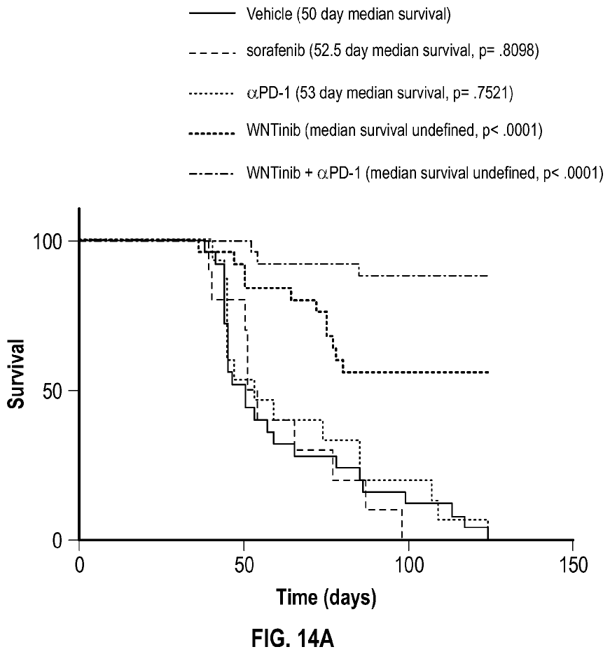

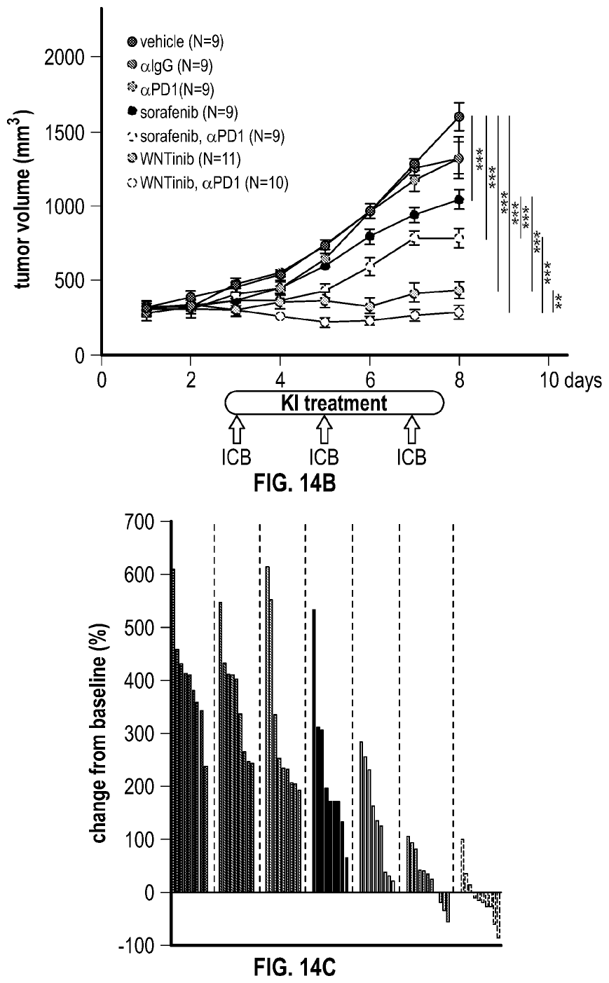

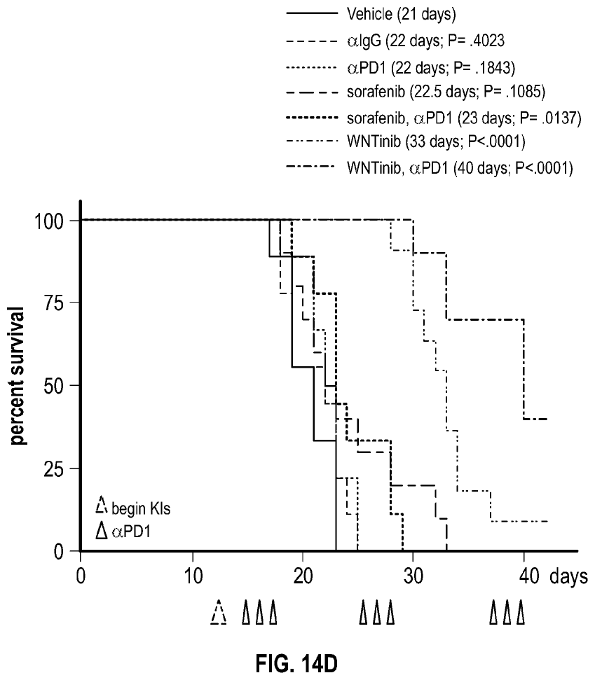

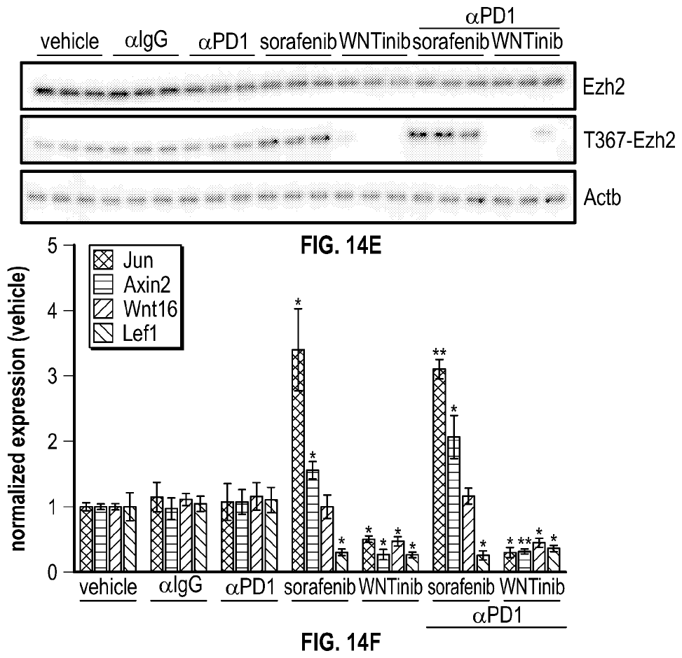

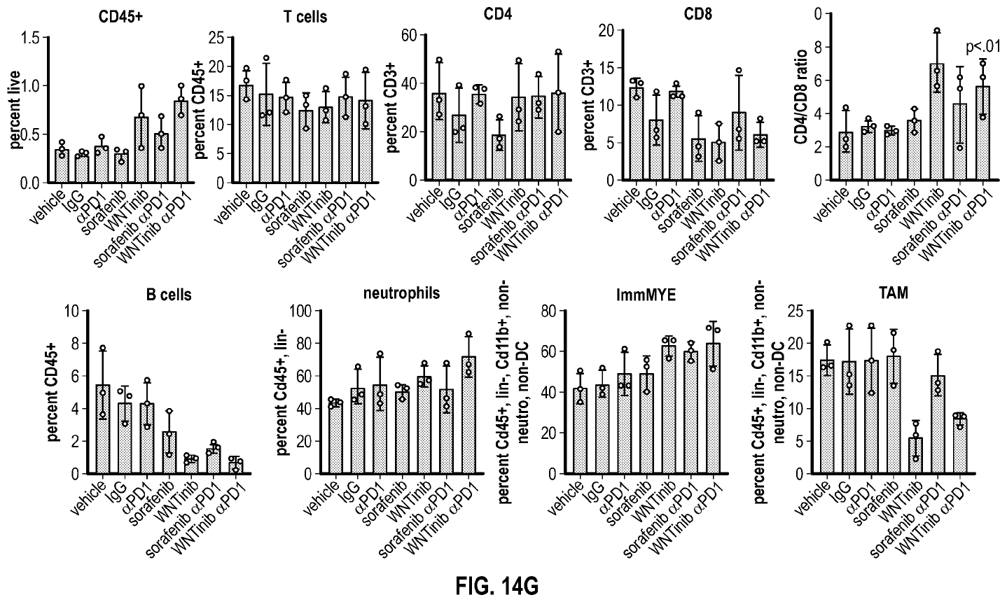

Given that tumors with dysregulated WNT signaling are associated with tumor immune exclusion and overall are refractory to treatment with immune checkpoint inhibitors, the efficacy of WNTinib was tested in combination with immunotherapy. First, a hydrodynamic tail vein mouse model of MYC-lucOS, CTNNB1 mutant HCC, which is known to be refractory to treatment with anti-PD-1 was used (Ruiz de Galarreta et al., “O-Catenin Activation Promotes Immune Escape and Resistance to Anti-PD-1 Therapy in Hepatocellular Carcinoma,” Cancer Discov. 9(8):1124-1141 (2019), which is hereby incorporated by reference in its entirety). C57/BL6 mice were randomized into treatment groups using IVIS imaging to quantify tumor size based on luminescence signal intensity. After randomization, kinase inhibitors were dissolved in 25% cremaphor:ethanol in water and administered orally (30 mg/kg sorafenib, 20 mg/kg WNTinib) beginning 7 days after injection following a 5 days on/2 days off dosing schedule. Immunotherapy (anti-PD-1) was administered via intraperitoneal injection on days 11, 13, and 15 after injection at 200 kg. As evident, a synergistic effect of the WNTinib+antiPD-1 treatment, compared to vehicle control or sorafenib, was observed (FIG. 14A).

Next, the same combinations was evaluated in MYC-CTNNB1 tumor organoid allografts treated with vehicle, anti-IgG control, anti-PD-1, sorafenib, sorafenib+anti-PD-1, WNTinib, or WNTinib+anti-PD-1, respectively. This experiment independently confirmed the superior efficacy of WNTinib as a single agent compared the standard of care and the synergy between WNTinib and anti-PD-1 (FIG. 14B). WNTinib and WNTinib+anti PD-1 were the only two treatments able to induce tumor regression (FIG. 14C) and prolong survival (FIG. 14D).

Importantly, WNTinib in vivo target engagement was measured. The reduction of WNT target genes (FIG. 14E) and the reduction of pT367 EZH2 (FIG. 14F) in tumors treated with vehicle, anti-IgG control, anti-PD-1, sorafenib, sorafenib+anti-PD-1, WNTinib, or WNTinib+anti-PD-1 was measured, respectively.

Next, the effect of WNTinib on immune cell tumor infiltration was evaluated. WNTinib and WNTinib+PD-1 treatment were able to significantly increase the number of CD45⁺ cells, the ratio of CD4/CD8, and the number of neutrophils and reduce the number of B cells and immunosuppressed tissue associated macrophages (TAM) in tumors, compared to vehicle or standard of care treatment (e.g., sorafenib) (FIG. 14F).

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed is:

1. A method of treating a tumor having a dysregulated Wnt signaling pathway, said method comprising:
contacting a tumor having a dysregulated Wnt signaling pathway with a compound of Formula (I) having the following structure: 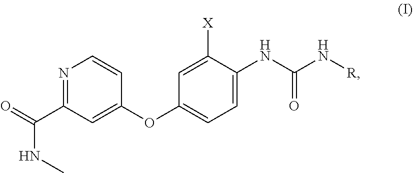
or a stereoisomer, pharmaceutically acceptable salt, oxide, or solvate thereof, wherein
X is a halogen and
R is a phenyl substituted with a perfluoroalkane
under conditions effective to treat the tumor.

2. The method according to claim 1, wherein the dysregulated Wnt signaling pathway comprises a mutation in one or more genes selected from the group consisting of CTNNB1, APC, AXIN1, AXIN2, GSK3B, LGR5, RNF43, ZNRF3, LRP6, FBXW7, TCF7L2, and combinations thereof.

3. The method according to claim 1 or claim 2, wherein the dysregulated Wnt signaling pathway comprises a mutation in CTNNB1.

4. The method according to claim 3, wherein the tumor encodes β-catenin comprising an N-terminal phosphodegron mutation or exon 3 indels.

5. The method according to any one of the preceding claims, wherein the tumor is associated with a colorectal cancer; a gastric cancer; an endometrial cancer; a lung cancer; a liver cancer; a hepatocellular carcinoma; a hepatocellular adenoma; a hepatoblastoma; a melanoma; a bladder carcinoma; a pilomatrixoma; an ovarian cancer; a medulloblastoma; an adenocortical carcinoma; a pancreatic cancer; a NSCLC; a liver adenoma; a LIAD; a hepatoblastoma; or a cancer of the uterus, pancreas, prostate, stomach, bladder, anus, or esophagus.

6. The method according to any one of the preceding claims further comprising:
contacting the tumor with an immune checkpoint inhibitor.

7. The method according to claim 6, wherein said contacting the tumor with an immune checkpoint inhibitor is carried out simultaneously with said contacting with a compound of Formula (I).

8. The method according to claim 6, wherein said contacting the tumor with an immune checkpoint inhibitor is carried out sequentially with said contacting with a compound of Formula (I).

9. The method according to any one of the preceding claims, wherein in the compound of Formula (I) X is fluorine.

10. The method according to any one of the preceding claims, wherein in the compound of Formula (I) R is a phenyl substituted with C₂F₅ or C₃F₇.

11. The method according to any one of the preceding claims, wherein the compound of Formula (I) has a chemical structure of

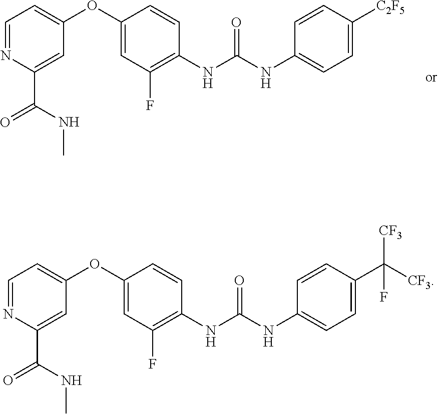

12. The method according to any one of the preceding claims, wherein said contacting with a compound of Formula (I) is carried out in vitro.

13. The method according to any one of claims 1-11, wherein said contacting with a compound of Formula (I) is carried out in vivo in a subject having the tumor.

14. The method according to claim 13, wherein said contacting with a compound of Formula (I) is carried out by administering the compound of Formula (I) to the subject.

15. The method according to claim 14, wherein said administering is carried out orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes.

16. The method according to claim 14 or claim 15, wherein the subject is a mammal.

17. The method according to claim 16, wherein the subject is a human.

18. A method of treating a cancer having a dysregulated Wnt signaling pathway in a subject in need thereof, said method comprising:
administering to a subject a compound of Formula (I) having the following structure: 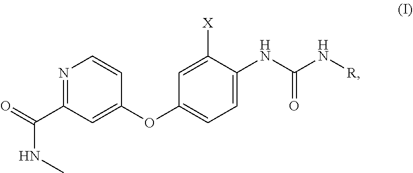
or a stereoisomer, pharmaceutically acceptable salt, oxide, or solvate thereof, wherein
X is a halogen and
R is a phenyl substituted with a perfluoroalkane
under conditions effective to treat the subject for the cancer having a dysregulated Wnt signaling pathway.

19. The method according to claim 18, wherein in the compound of Formula (I) X is fluorine.

20. The method according to claim 18 or claim 19, wherein in the compound of Formula (I) R is a phenyl substituted with C₂F₅ or C₃F₇.

21. The method according to any one of claims 18-20, wherein the compound has a structure of

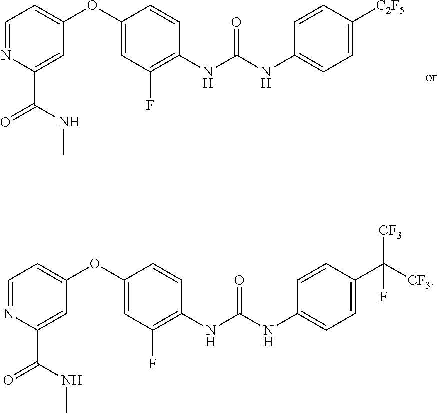

22. The method according to any one of claims 18-21, wherein said administering is carried out orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes.

23. The method according to any one of claims 18-22, wherein the subject is a mammal.

24. The method according to claim 23, wherein the subject is a human.

25. The method according to any one of claims 18-24, wherein the subject is treated for colorectal cancer; gastric cancer; endometrial cancer; lung cancer; liver cancer; hepatocellular carcinoma; hepatocellular adenoma; hepatoblastoma; melanoma; bladder carcinoma; pilomatrixoma; ovarian cancer; medulloblastoma; adenocortical carcinoma; pancreatic cancer; NSCLC; liver adenoma; LIAD; hepatoblastoma; or cancer of the uterus, pancreas, prostate, stomach, bladder, anus, or esophagus.

26. The method according to any one of claims 18-25, wherein the dysregulated Wnt signaling pathway comprises a mutation in one or more genes selected from the group consisting of CTNNB1, APC, AXIN1, AXIN2, GSK3B, LGR5, RNF43, ZNRF3, LRP6, FBXW7, TCF7L2, and combinations thereof.

27. The method according to claim 26, wherein the dysregulated Wnt signaling pathway comprises a mutation in CTNNB1.

28. The method according to claim 27, wherein the tumor encodes β-catenin comprising an N-terminal phosphodegron mutation or exon 3 indels.

29. A method of treating a tumor, said method comprising:
contacting a tumor comprising cytoplasmic EZH2 with a kinase inhibitor compound under conditions effective to treat the tumor.

30. The method according to claim 29, wherein the kinase inhibitor compound is a compound of Formula (I) having the following structure: 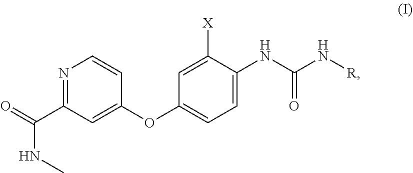
or a stereoisomer, pharmaceutically acceptable salt, oxide, or solvate thereof, wherein
X is a halogen and
R is a phenyl substituted with a perfluoroalkane.
